SEQUENCE LISTING (97 sequences)

SEQ ID NO: 1 - DNA, 20 bp, Oryza minuta
tcccaaatctcaggtgtctt

SEQ ID NO: 2 - DNA, 18 bp, Oryza minuta
atcagcaggcggcaaact

SEQ ID NO: 3 - DNA, 20 bp, Oryza minuta
gtaggtacatcaaggacgag

SEQ ID NO: 4 - DNA, 20 bp, Oryza minuta
agcatatctttgagcatttg

SEQ ID NO: 5 - DNA, 19 bp, Oryza minuta
actgttgtagcggaggaga

SEQ ID NO: 6 - DNA, 20 bp, Oryza minuta
tttccattgtrggcgaggtg

SEQ ID NO: 7 - DNA, 20 bp, Oryza minuta
atgtcagccaaaatcaatca

SEQ ID NO: 8 - DNA, 20 bp, Oryza minuta
ggaaggggacaaggacaata

SEQ ID NO: 9 - DNA, 19 bp, Oryza minuta
  misc_feature (12)..(12): n can be a, t, c or g.
cttttcgtgggnggggtta

SEQ ID NO: 10 - DNA, 20 bp, Oryza minuta
ggtgagtgatgacagcaaca

SEQ ID NO: 11 - DNA, 18 bp, Oryza minuta
cagctttgaggacattcg

SEQ ID NO: 12 - DNA, 20 bp, Oryza minuta
ttttgaccccagacgaccag

SEQ ID NO: 13 - DNA, 20 bp, Oryza minuta
gagaggggatggacaaagag

SEQ ID NO: 14 - DNA, 20 bp, Oryza minuta
ggaaaacgaaacggtagatt

SEQ ID NO: 15 - DNA, 18 bp, Oryza minuta
tgccgcctgctcgtcctc

SEQ ID NO: 16 - DNA, 18 bp, Oryza minuta
ggttgttttcccttgtcc

SEQ ID NO: 17 - DNA, 19 bp, Oryza minuta
aaggaagatgagccgtgat

SEQ ID NO: 18 - DNA, 20 bp, Oryza minuta
tgatgcgtgatgattttgta

SEQ ID NO: 19 - DNA, 20 bp, Oryza minuta
tctgatgtcctctgaactga

SEQ ID NO: 20 - DNA, 20 bp, Oryza minuta
actgcctctgctgttgttga

SEQ ID NO: 21 - DNA, 22 bp, Oryza minuta
ggtctggcactatttttacttt

SEQ ID NO: 22 - DNA, 20 bp, Oryza minuta
gcaaaaggaactgataagat

SEQ ID NO: 23 - DNA, 22 bp, Oryza minuta
atctcatttttaggttctgtcg

SEQ ID NO: 24 - DNA, 20 bp, Oryza minuta
caagcagccaccaccatctc

SEQ ID NO: 25 - DNA, 18 bp, Oryza minuta
cgaccgaaccgcctttag

SEQ ID NO: 26 - DNA, 20 bp, Oryza minuta
tcaacgaagaagagatgtag

SEQ ID NO: 27 - DNA, 22 bp, Oryza minuta
aatacacaccccaatcatactg

SEQ ID NO: 28 - DNA, 22 bp, Oryza minuta
aaaaacaagacggcagaacaga

SEQ ID NO: 29 - DNA, 20 bp, Oryza minuta
aagaagataatggaatgggg

SEQ ID NO: 30 - DNA, 20 bp, Oryza minuta
gagagattgcagagaagaga

SEQ ID NO: 31 - DNA, 20 bp, Oryza minuta
tttccttcttgtaacctgat

SEQ ID NO: 32 - DNA, 20 bp, Oryza minuta
ctgcttgctattcgttcatc

SEQ ID NO: 33 - DNA, 20 bp, Oryza minuta
taacagttctcccaatctcc

SEQ ID NO: 34 - DNA, 18 bp, Oryza minuta
ccggactaagtactggct

SEQ ID NO: 35 - DNA, 20 bp, Oryza minuta
gccatgttgctgcggaaaat

SEQ ID NO: 36 - DNA, 20 bp, Oryza minuta
aaacttagggcattcaatcc

SEQ ID NO: 37 - DNA, 19 bp, Oryza minuta
tggtgcactcagaaagaat

SEQ ID NO: 38 - DNA, 18 bp, Oryza minuta
cgccatgttgctgcggaa

SEQ ID NO: 39 - DNA, 20 bp, Oryza minuta
agatcccgccactaaactta

SEQ ID NO: 40 - DNA, 20 bp, Oryza minuta
cttgttggtatgagtattct

SEQ ID NO: 41 - DNA, 19 bp, Oryza minuta
gcagtgtcatcttgtctcc

SEQ ID NO: 42 - DNA, 20 bp, Oryza minuta
ttgaatacagtgctaaagtg

SEQ ID NO: 43 - DNA, 20 bp, Oryza minuta
gtatgacaatggatggagaa

SEQ ID NO: 44 - DNA, 19 bp, Oryza minuta
tagcaagggatgggagcaa

SEQ ID NO: 45 - DNA, 20 bp, Oryza minuta
taggtgcacaggaagagaat

SEQ ID NO: 46 - DNA, 20 bp, Oryza minuta
tcggtatttgtttgattgga

SEQ ID NO: 47 - DNA, 20 bp, Oryza minuta
cgcgtgatattcttgactgt

SEQ ID NO: 48 - DNA, 20 bp, Oryza minuta
attaggcaccccaggcttta

SEQ ID NO: 49 - DNA, 20 bp, Oryza minuta
tccttgtggcgatttgtatt

SEQ ID NO: 50 - DNA, 19 bp, Oryza minuta
tccaggtaaagagaaaagt

SEQ ID NO: 51 - DNA, 20 bp, Oryza minuta
ttgtactaaatcagaagcac

SEQ ID NO: 52 - DNA, 20 bp, Oryza minuta
tgttggcatcttttatctga

SEQ ID NO: 53 - DNA, 20 bp, Oryza minuta
tagctgctatttgtgatgta

SEQ ID NO: 54 - DNA, 19 bp, Oryza minuta
gcacgcttctgtaactcca

SEQ ID NO: 55 - DNA, 20 bp, Oryza minuta
attagatttggcgattatgc

SEQ ID NO: 56 - DNA, 20 bp, Oryza minuta
tacacaccccaatcatactg

SEQ ID NO: 57 - DNA, 20 bp, Oryza minuta
gcatcagcaggcggcaaact

SEQ ID NO: 58 - DNA, 20 bp, Oryza minuta
atagagtgagatggagaaga

SEQ ID NO: 59 - DNA, 20 bp, Oryza minuta
ttaaagcccgtcaccgatag

SEQ ID NO: 60 - DNA, 19 bp, Oryza minuta
gcaccccaggctttacact

SEQ ID NO: 61 - DNA, 20 bp, Oryza minuta
  misc_feature (5)..(5): n can be a, g, t, or c.
gggcntaatttcagttcttt

SEQ ID NO: 62 - DNA, 20 bp, Oryza minuta
gtactggtgtgattatggtg

SEQ ID NO: 63 - DNA, 19 bp, Oryza minuta
aaaaatacaaagaaaaacc

SEQ ID NO: 64 - DNA, 19 bp, Oryza minuta
caggctttacactttatgc

SEQ ID NO: 65 - DNA, 20 bp, Oryza minuta
cgatacgtgtttttctggac

SEQ ID NO: 66 - DNA, 20 bp, Oryza minuta
gcacattaacaacaaagaaa

SEQ ID NO: 67 - DNA, 20 bp, Oryza minuta
atgttcagttggagagtagc

SEQ ID NO: 68 - DNA, 19 bp, Oryza minuta
aggtgttcgccccgcaggt

SEQ ID NO: 69 - DNA, 20 bp, Oryza minuta
cactgttgtagcggaggaga

SEQ ID NO: 70 - DNA, 22 bp, Oryza minuta
cagtacgcgattttcattgttc

SEQ ID NO: 71 - DNA, 21 bp, Oryza minuta
cgtgctgatgtgatgcttcta

SEQ ID NO: 72 - DNA, 20 bp, Oryza minuta
ttcttcgtctttatcgcaac

SEQ ID NO: 73 - DNA, 19 bp, Oryza minuta
ggtgcagcggggaggaggt

SEQ ID NO: 74 - DNA, 22 bp, Oryza minuta
ggtgccctcctcaaatcttatc

SEQ ID NO: 75 - DNA, 22 bp, Oryza minuta
ttggcgtcttgaggagtcgtat

SEQ ID NO: 76 - DNA, 20 bp, Oryza minuta
ggcggtggtggcgaggagtt

SEQ ID NO: 77 - DNA, 19 bp, Oryza minuta
ggtgcagcggggaggaggt

SEQ ID NO: 78 - DNA, 22 bp, Oryza minuta
atgtgctgcaagaggaagtgaa

SEQ ID NO: 79 - DNA, 20 bp, Oryza minuta
ttgctccatcttcctctgtt

SEQ ID NO: 80 - DNA, 20 bp, Oryza minuta
atggtcctttatctttattg

SEQ ID NO: 81 - DNA, 22 bp, Oryza minuta
atacagaccagagaaagaaaaa

SEQ ID NO: 82 - DNA, 22 bp, Oryza minuta
acagaggaagatggagcaaagt

SEQ ID NO: 83 - DNA, 76272 bp, Oryza minuta
aagcttcgggcagctcccggtggcgcggcgatggctgcaggctcccggctgcacgacgacaactcccgcgcccgcgtcgaccgctgcaggcgccaggtagcgcgacgctcctggcggcgtgtcgatagagggggcgggacagtagcggcgcgcgcgagaggaggcgacggcgcgcaaggtggctggcggtggtggagtggtggtggacagcggtgcgccgtgcgcctggtggtggtggttgtgtgggcgaccgcgtgtggctgcagcggagagccactggaggagaggcgctcgactcgatccggaggagtttgaccaaagactagggttagcttttatatatacctgttaattgggcattagtattatgggccaaaacgtctaaaaagtctagatttagtcatctatacagtaaagtcgggtcaccgcgggggcggggacggtgaatggtgcaccgtccccacccccgtcatctccgatggtgctaactttggaaccattttattcaccattgtggatagatattaaccatcccagtcacctaaaaggtgaattcaccgtgagaaaacggtgaacggggccccattgccatctcttctttatatgtacatgaaagctagggttggagcatattgctcatgcctaaatccaaagatacaaagcctatccaaaatggctccatattcctctgcattctccatccatctctccaaatttagctccatatttttttaatgacccatgactctgttttcacctaaattggctaaatacttgtcttacttttagttatttatgaatacaatatgttaattactcttaattattatttttataaattagttgctcttaatgtatatatatatccaactactataagatagggagtgagtttggatcccaagatttaaaaaggaagagggggggggggatggacacagagtaggataagatagggagtgggttagggatatggatagagggggatttttgggtgtttctcttaaagagaaaagtctatatttagttatgcaactatggctttggtatgtgtctaaggctctgttcatttctggaagttcccaacctccacctcccatttcccgccgcatgctttttaaactgttaaacgatgtatttttaaaaaatatttaggaaagttgttttaaaaaaatcatattaattcataagttttttatctaatacttacttaacaatgcgttaattaccgttttgttttccgtgcgtggaggattagttccaacccaccttgaagaacatagcctaaggcggagtttgtttcagcttttaggctgaaattttgtgatgctacgtgaaattttgacgagataagtcattagcacgtgactaatcgaatcttaattattacaaaattaaaaatatatatttatacataataattttgttaaatgaaacgtaccgtttagcggttgggatgcgtgctcatagacaaccatgaagtagctattccaaaacattgcttagaacgcaccctaaaaaagaccatggctttggtccacttttgatttctaaatcatgaactcatttacttttggtccatgaactctcaaaaccgttcgcatttcaccccataagttcaaaccaggattgttttcaatgatgtaacgtcaacatggagttgaccagggtgatgatgataaaataaacaatagcaaaataaagaaaaaaaatactgtgacctacatatgtcaaacttgcttcctttccttctctctccataatcctatatatgttatttactttaaattgctacctaaaatgacacatgatgccatgtccttaaaaaccctaccgtgattatgtcaaggtttgaaatatgaggcggttataagagttgaggggctaacagttacagggtttcatagttaagtgaactttttttcttcttaaaaaacataacagagagtgcaaaacagaataggaagtgtgatatagaggtggattgtattgcctctactaaacgaatctggaattttacattgtagaccttttaaaaacttatgttacaaatagatggctagaaaaatttatttctagaatgagccctttttgcggcaccaacgccattattactatggtccaacatgatggcaccaatgacattattgccatctcccttgccccttaaatggaagctaagggctagtttggttcgtggccttaattgaccttgccaatatttgtcaatttcaatagtgtttagtgtctatttggttataagccaaattttgacatgcctaaagaaatagaccatttcaatagtgaaattaggttgttttgcttcaatccaaacacaactttatcttaccaagattagccatgccaaaacttaccaaaatttgacattgacaaaaattggtaaggtcaatttaggtcacaaagcaaaacagccctaagtctcggatgtgaaggggttacgttcagaacctatttgtaaaaaaattccaaaatactaaaatataaattatgtttcttacatataggtgtttaatgttaaatgtaaccgaggacctatttataattttttttataaactattcattcttttgaatttcttttataaataagttctcgacgcaagtgttattactgttgacgcccttcatattagtgatttagcttttacggtgaagagatacaaggacaattatgctagccccgcgaaaatgatccatttctagaataagttttttcaaaaatttatttgtaaaataatttttcgaaaataacaaaagtaaaagctgctaaacgaagagttcctgcatttcaaacaataataaactattaggaaaaacacgtgatccggtgacaacactagtttactcctctgtgcacgtgggtccaggcaccggttaccgtttggtgttgtcctgttccggttcctcggtgtccagtagttccactgggtggttggatggtccgctgagcgcttggggcccacgcgtagccttgttctctggataagaaaactgcacttccatttctcgtgaaaatgtctactggtagaacagagtatgggccggcccactaacttagcctaagtaacatacctcaatgggctctcatagcccaataccccactgctgatttttttcttagggcatgtttaaattgctggcaaaataaacctttcgaaaaaaaaaatattaagtcgataacattatcaagttttgatagggtaagtttttttttgggatgtgtttaatttggtgcccacatataggggtggaaaaaaaaagctcgaagctcgcgactcgaattgtctcgtttcaggctcagcttggatcgactcgagttcggagtcttaacaagtcgagccgagacagctgttttgctcgttaagctaacgagctgatcccgagccagctcgcgagtcactttgtaggctcgttaaactctcatagcccaagaatcctacaggccacagcccaatcaggtggccgaacccagtaaaaactccctccccgacttcctgttttctctaaaccctatcctatcttccgcctccaccgaatcgcaggctcgcaggcggcgacttgcaaccaccaccaacaccaagcaggctgttgtcgtcgccttctccttgtgccgtcggagcgcgcccaccggctgctcctcccgcagccgcagggccacgctgacgacgccgctcctcccgcagccgtagcaccatgccgaccacgccactcctcccacagctgcaaggccactcgctacctcctagtccctggaggcttgaaccctgctggtatgcttcatggatcaggagcgccggttagcaagcatggaatccaagaaccgtagcaaggggaagaaattgtcaggagcttctccctctagtttgactccagccggcagccacctcgcctctgttcagaagccaccaagacagagacctaaagcaagtccaccaacctcacatcaagcaagtgtcgcatcaatatttttcacaatttgtagcaattttttttcctaattttgtcactgattgaaccattcaacttgtgatgcacaatctgtagtaagtgttctgtaggtttgttttcttattctgtggtgcactgatgatggacatagttatatattgaattggattatacaaggacccaatggagggttcattcagttaacgagctaaacaagctcaatgagccagctcgagcaagctcgctgagttgagtcgagtcagcatttcagctcgttaagcctaatgagccgagtcgagccgagatggctttttaaggcagcgagccaaaccgagcgagtcgagctgcctcgatatccacccctacccacatataggccatcaaagtttggcaacatttgaaggttatgatattttattgtgacgttgctaaattcctgtgagcattaccaacgtttgtttacaaactaaatgtattaatatatcatttttctaaaaaaatgttacgattttaaatgtcatcaatatgaataaagcccttaatctctcatcctactggctgccgtggcacctaatagcacatggcatgtccacgttggcaacgagcgcgccacgtaagtatgtgatgctgcgaaaaggacaaggccagacagagtcacagagctagctgcgtccagctaagcctcgtgcacgtaccgattccaatgccttctcctggaggtaagtatttttgttccacttgattatatttctttggtagatgacgtggacgctgccacctggatgctctggaatccagcgaagtcagcagtcaaattaacaggtattttacaatttttttttctgaattattgtggtcgtgttgcatcggtaagagtaacaccaagcttaactttccttgatcgatgctgcttttactcaaacctggaagagatagtaatattttaattaatcagtgagaaaagaaaaactgatgtgaacgggacaatttgcacgatgggggaataataatattatttgggttgggttttgttaaatactcctacttgctttgtcttttttctcgaccacatgaccaaatcgagctgtcacaactcgcacgtccataaattaagaatattatgctaatgcagttgacattcgacggcaactagcaagacggagtagctcaccaaatactattgctatatatctaattcaaccagcttggataaatggtgagctgaatgcttgctcaaacactcagactcaccaaccatattatcatgtggctgtctatccagattgtcaagattttggtcatgtgatatatgatatctctataatgcatgcatgcctttttaatgaatagttcaatttcttacttttgcaggccaatttgttgtttgtgagtggtcaatctaaaattaaatctaagataactttgataggatggatgagtagatttttttttaactaaaatgtgaaattgatgtcaaatttgcatgataagttgccgttccaacgaatagcactagtaaaacacaatatttcgtgaatgcatgatgtttgaaaatgatgaaggtaaagattaagttgacgcacgtaaaacaagaaagtcattagtgtataattaattaaattttaattattataaatttgataaatggatatatatgatattttaaagtaagttctataatataaagtttttgcacggaatgtacatatagtagttttgataagtgctaatgataaccgagctaaactctataatcctaatcatagaacagttcgtttagtgtgcatgagtagagtaggaactcatgtgctattcagccagtttagtcctaccacgttcgtttggagaacaaaggagtgagtttgtttcgttttccgcgcgtacgcttcccgagttattgaacggtgcgttttttgcaaaaattttctataggaaaattactttaaaaaatcatattaatctatttttgaagtttaaaatagttaatactcaattaatcatgcgctaatagctcacatcgttttacgtatcttcccaatcttctcctcactcacaaataaaaaaccaaaacatgaagttagtatacctcaactaattaggttcctgatagtgaaacccatccatccagatttaagttttaaaattagcacgggtgcttgtatttacgagtagttattctaccaaccaagttgtagacttgatacagtcgagttctagacttcgcatatcaagatatgtcggattagtcttcgaaagtacttatagaggtagggtgtcttagacatatatccgtctagtctttgaaaatgtttatagggatggagtgcgtatatgtgtacattcataagagtgatagtacgcgtgtttatatcagctgcgactgtactatattttaaaaaaactaatgcatcctcatctaacgaactaaacctaccagagaggggaggaggaatggtaggaacactaaaaaccgaaaacttttagtggaattaaaagttaccttcgaagctgatggagatcggtctgaccggcttgattgccctagtttgaccgcgccttgttgccaatctaaccggtgtagatcgaggtctgaccgcccttcgcgccatcgacgcttgttgccgccatataactgccggtgtgaccgagcagttgcctctggtctgatcgctggtgatctgtcggtgtgatcgctgcatctgagcaaaacacaaattaaaagatgtcttgaaagttcgtagattgaattttattgcttatattcgtgttacaaagtacaacaacaacacttctctcacagaattcgactaaactcgaaaccttaacttttctctaaattaaactctcccaaaatcgatataaggatatctcaactccctctctatttatacccaagacaagcagcctaaagtcacgaatctaatttgtacaagaagttctaattcactagaaaaccttcccgtacaagaaacaaacttatctcatcataattcgaatttcaatcctcccaaatttagactccttccaagtttgactccgcttttcatacgcacacaatctcccattgaaacggtcaattaggcctaagggggggggggtgaatgggctaatttaaaaacttaagtaaatgcagaagcatggtttttcagaaattctgaaaatgctttacgaaaattctgaaaatcacagaatatgcacaagtgaaagtaaattctagatctagctacatacaacaatgaaatacaagcacaaacaacaactagacttataacggtacaaacaagcaaagctagaggagggagagaggtatatcaccgaggttgttgcaataagagttgttcctgaagtttgaatccttaaggggattatactttccgttgaggagctcacaacgagccgggtctttgctaacccttttctcaaggggttgcccaaagcactcctccttccactagtggtatctaggggtgaaaacgatcggaatcgttagcatctcttcggaaacgatgctcgatcggccggttagtcctcacatatactcaaacgaaaatgttagtcctctctaatcatattgttattaatcactaaaatcattaggggcctagatgcactttcacccattttatgcgacaaagaatcatcataaaacaatgtgcattgttctttagactaagcatcccgtacgatattctgattgtccagacatcatcttctcccaagttgactctcgatccatcaccgacaacgctctcctgaggcatcaagacacacttacacaggaatcaaaacaagaaaccatatccgagcacaagttttttcctaacttgactcgacattagcaaacaacaatattacacacatatagaaataatctagaagtcataattatgagataaacatgagtatccaaataaacaactcaaaaccgaccctaatcaggatccagccagtctgcccgcacataccacaccggtctgaccggccttaccggtacagtctgatcgacttcacacaaaaaaaacacttcaccaataatcaccaaatactaaattaattatctatcatgccaattattcatcacaaattaataataaaagcacactttgatttcacacaattagttaactgacagtcatatcgatatatagccaaatatcggcaataattgtacaagctccctacatctattctcttaggttctcctagatcgatcacactggtctactccttccatcccataaaatacaaacgttatcccaaatgtgagacaccataatactgcaaatctggagaggcgtgattaataatttctcatcagccataggggtaataataatccaaggctgtgtttagttccacgtaaaaattgaaagtttaaagaaattggaacgatgtgacgaaaaagttggaagtttgtttgtgtaaaaaagttcaatgtgacgaaaaagttaaatgtttgaagaaaaaagtttgactaaacagggccaagtcatggtgatggtaagtcagcccctaagctttcactttgacctattctactctcacactgacgttgacattgtatcacagggcaccacccattccctctccttaatttttgtttgactatagtaaccatatcaatcaaataggtgtgaaagctaacctagattatttctgatcacactgatcacattctcactagctattctacactttctaccttcacataggacgacaagtgtaccaacctactgataaattttcaagcctcatttttcttataagttatggccaaaattaaaattttaaaaattagttttaaattattttgatgttgtttcatcacctttttctagctttagctaaaatgatataatgatagagaaaaataaaagtcataacctaaaaataatttttagctcgttttttatagcttattagcagcagactattcaactcgaccctccgtgttttctcatataagcccataccatggctacgtttgaaactacaactctacaagttctcatattttattttcttatttttcacaagtacgtttttgaaactgtaaaacgacgtgtgtttctaaaattttctatagaaaaattactttaaaacatcatataaatttattttaatttttttaactaatactacctctgtcctagaaagactgcagttttgcactattcatatccaacgtttgaccgtccgttttatttgaaagttttttataattagtatttttattgatattaaatgataaattatgaataatattttatgtgtgactattttttaatttttttaataaattttcaaacaagacggacgtggttagtatttttattgttattagatgataaattatgaataatactttatgtgtgactatttttttttaaatttgttttataaattttcaaataagacagacagacaaacgttggacaccaaaattcataactacagtcaaaatgggacggaggtagtaattaattaaccttttttaagaagaaatgagaagttcgaacacaacccgcacacccaaaccaagggggtgtttagattgaggggtgtaaagttttggcgtgtcacatcggatattatatatgatgttgtatagggtgttcggacactaataaaaaaatcgacttattaagcctaattaatccgtcattagcatatgtttactgtagcacaacattatcaaatcatgtagcaattaggcttaaaagattcgtctcgcaaattagtaacaatctatataattagctattttttagcctatatttaatatattatacagatgtccaaacgttcgacacgacgggatctaaaattttcgggtgggctcatcgaaagcaaacaaaacgaacccccgcacacgaacggtcacacgctgatcttttctccaaacccaaaaaagaaaagaaaatcagaaaaagtcccgccactacctctactactgccggccctacccccacagattccaggcgccagctaagcacacaccgacgtgcacccctcccctcccgcatgcgtttccactctgtctccgctccacacccgagcccggtcaaacccaaccccgccgcgccgtcactccgcacccgactcactggcccccaccacccaccaccgccgctgccccgtgggccccgcccccacccgccgacgcgtgggcccccacacgccgccggccgcggcgggtcacatgcgcgtcgtccaaactccgaggcgcgcacgaaaaagagagagacaaaaaaaaagaaaagagaaattctatttaggcgctctaggtataaaaatctactcctactcatcaactacttttccacttcgattcctctccccttccccctcctcttcctccctctcctctcctctcctctccgccgctgccgctgctgctgcgtgctcctctcatccccgtctcttccccctccgcgcgcgccgcccactcgctgggaggaggaggaagaggagaccttccccggaattcgtgctcgccggatcgggctcgccgcaatccatgtcggtgagtggtgctgttgatgtgtttcttttttctactgattttagagttggtgattggttgcgctgtctagatcgagctgacccgcctgcgtgttggattgtattgattagaggaagggaggctgattagcacgaggtgtggggaaaaattagttgtaaaaaaaattggaggggttaatagatgggcgtttgttaagtgacgtaaggcgaaagtgatgttatgctgttctgggttagtaaggttcttggcaatcagttttggtcaaatcttactggttcataggtgttttggtcgaatttcagcttcagatgaagtttgtccctcgtttccaggattgttggcggtcagttcttcatcgttaggcatttaattggttgaacaggaaattggggtcatggtagaatgcgaagtttctgaaaacataagtagagaacaaaagtaggagaattttgtcaattaggtacgatggaaggtcaccgtcagcttgttcaactactgttaggttcaaactttgatcgtgcttgagatttctttgctaaaataaggggatatgtggcaaagggagaataaaccatgatatgatttttgttactgactatagtggctgtaaatgagaacttgtggaatcacagaaagttccaaaaaacaatgcagaaattgtatccttttttgtgctttgcctagtccattttctgacttctgagatgaggtccccgcatcacatagaactgcaattgcaattcaatgcattcatgcaacaaacagacagactactgtcctataacatgagttatgacttacgagtagccttgcatattgtacatgcataagccaaagctgcttgtttattgtaaaccagatactctgtaccataaaaaccataatccattaagttttcttgtttactttgttgaacattatactagatacctatggtgttgactgttacataacattctgttcaccatttggcaattttccccccttagtgacattcattcttttgagatttcagtcatgccttccacatgacaatatgttgcattcaactatgatgtttggaattgggaaacggtactcatacttaatgtttcttttactaattctgattgaaagcatgcaacacaccttttaatccacatgcacatacatcaatcaatgctttggagagagtgcatcatggatcaatcatattgatggtttattagtaaacattttacagcaattatttgtgcacatggccctcatacaatttttgttgggcattcaacccttgttttgaggtgttaaataaattattttttattatgttgtcaaattgcctgacctttgtattggtggttgggcatctagctgtgcactgtcaataactcctggcggtgctcctctttttcctggctgttgaacttccaatctgaccattcattatgttcatatcttgtacacatgtggtgagatgtcaattgctgtccattgtatgttattatattttacttagcattgttcaactgtaatagacagtgatatatcatacatatcaaatgaaaccaacacagacaaagcatatgctgtgttgtcatcttcttatttcttgtactgttctgttctgtcttgaaggttagctgtgttagtcatggcccactatggattctcaaaaggcatcctttatgttcattagtttcatctttttctattttccaattttattagatggctatacacgactttgttcacacataatctctaattatcaatcgtctggtcatgcttggcaggcctcaatcaaatgccagcccacccatgggaaatgggccaggattgaggcctgccgaacaaggtgtagaggtagatacaccctttaatgtggatcatggttcctttctaggaggaagcaggtcgacccttgcccaagtttggtgaatgggatgtcaacgacccagcttctgctgatggattcacagtgatattcaacaaagccagagatgagaaaaagggtgggaatgggcaagatactgattcaccctgcaaagagactaggactgagagggtggaatcatatgcccccaaaacaaactcggtatgtctattacattactcacgctgattataatatcagaatttcaatttctccctgattaatataaagttattccttttctcttgaactagataactagctagatgtcattaatctcattgttttcatgtgcagaagaaatggttttgctgtgtgacatccagtcctacacaatcttgatgaaaacgagttccatgggttgcaaaattactatcctttaattttgctatatacatactatccataagaccttgtagagatgcccagactctgctgtggtgcttgattgggcatctcttaaaactctgaggtgtgtgtatgtatgtgtgagggttatcagatgcacattcggataaatgaacttctgattgtaattctagccttccgtcctgtgacattctgtgatgtagtcgttattcagtgatataatgaatctcacctattgatataactgcacactgtttctcttgctttccgaggaaacaaacactgttttaccaaagttggctgtgctccaaatgaggattaaattccgtatgattctcaaattgcaattgcaattttgattttctcacccattaatatgtgaatttggaattttctttcacgtgtcaaattcgcatattgcaatttttctaaaggatacaagcacagtatagctcaatctcttgcaggctgagttggtagcaatgcaattggcgatgggagttttcttaatattgtaggcatcacatttctaacacacaatgcaacggtgtatcgactaggtgataaggtagcaaaggatgtgagatcgcaacagctcaatccttcagtttatgattgtcaaaatatcttgagtcttgctcaccctagaaaccttcgttatgcttgagatttgaaaaagagcaacgaattgtattacaagtcatgtgctctgtttctgaaatggaaataatactgacgagttctaaagaaaaaaagaaccatgcattcatgcctgtacaacagtagttggtactgccatgtgccatctgcttcagatgagtttcagaccctgcagcacggacagcaaatctactccctccgtctcttaaaaaaacatcctgcctctcctaatacaacgaatctaacatcacatcttttctaggctactttttttttttttggcgaagagagtacgtgtcagagtggagattgctatattggtttcagaatttcatcagtgtgcccatgcccaatactagtattattcgattttcagtgaggaacaaatgccagcatactaagggcctgtttattttgatgccattttcaaccttattaaattttgataaagttgctaaaaaagtggctatatttagtttgctgctaaattttggtaactatataagaaatcctgccaaaattttggcaagtctatgccctaatatttcgtgttgccaacacacaatttcacttttttttacacgatttagttcaggctgtgtttagatctaaagtttggatccaaacttcagtcctttttcatcacatcaacatatcatacacacacaacttttcagtcacatcatcttcaattttaaccaatatccaaactttgcgctgaactaaacacagcctcagtttctctgaaatttgcttgggattcattcgttcgagaagaggcgtgagatccgaaactttgggagagggaaagggcagttcaattgttcaaaatacctgatactatttgctcactagtagctcgcgtggagcttgagagagagagaggggagggagagtagtgtgagggtgggagcaatcgatggcggagacggtggtgagcatggcgatgtcggtgctgggcagcgccgtcgggaaggccgcctccgccgccgccgacgaggccaccctcctgctcggcgtccagaaggagatctggtaagtgcgtgcacgcttctgtaactccataccacatcggtgactctccatggcccctccgcggcggcgtgtcggcgacctcggcttccatggccgccgccgccgccgcctcacagccctgcttcgccgcaatttcttttttggtcttttggtgattccaacggattcaggatcaaaaatgaaacaatccggagagcctattcacgcttcgcatttcactaccccaaacttgtggattgtgaccatgcaaaccaaaattccttcagtttcatactttcatatatgctcaaatttccgttgttttcgtagaaacaaggttatttatcccacttacatcttgcaaaaaccaaagtacagttactcttatcgattttcatctatacacatgctagacgaatgcattgtggtccaattcaaatttctgatatattagctactaaccaagatgctcccatgccatgagcgcaagttccattgacaaggaaaaacacttttttatgatttgagcaagttccacgctggcgtcctcgttggcacaccacgtaagctaaagtggcaaaaaaaagaaaagaatttttctctctaaagtgagtcagtgacaaaaccttaagtgcaaaattaaaatgggtatttggataatcgccaaatctaaaagtggcaaatagttaaattccccttctatatttctctcttcagtggcaaaaacttaagtgcaaaaataaaatgggtatttgcataatcgccaaatctaatagtggcaaatagttaaattccccttttatattagtccttgaggagctacggcaatgtttgctagcaccggacaagatcatccaataaaaacttagagattatacataacagtgtcactgttactagcaaaattttccttcctattgcatggttagtacatttttttttgtgagaaatatatggtcaatgccatttgcaatatacctccaggctctagtcaatataagactacatacatatgcgatctacattttgtactaggtctctaccaaaagaaggggtttgtacccttctgttctcttaaattaatgagacgatttgtaggatctttctttttcaagggattgtgggagttctgactctcatggctaacgaggcgctctgaatactagtagtttatttccatccatctatttatttattttaataaattttattgcttttgaatttatatttgtaggtacatcaaggacgagctgaaaactattcaggcattcttaagagctgctgaagtaacaaagaagaaagatgacttgctaaaggtatgggcagagcaagtacgagatctgtcatataacattgaagattgcctagacgaattcaaggttcatgttgagagccaaagcttggcaaagcaactaatgaagcttggtgaacgccatcgaattgctgtacagattctcaacttaaaatcaagaattgaagaagtgagcaacaggaatacacgctacagcttaatcaagctcatttcctctataaccacagaggatgagagggattcctacctagaagatgctcgcaatcgatcaggtagcaacactgacgagtcagaacttgtgggctttgccaagactaaagatgagttgcttaaactgatagatgtcaatactaatgacggtccagctaaagtgatatgtgtggttggaatgggtggattaggcaagactacccttgcaaggaaggcatatgaaaacaaggaacacatgaagaacttctcgtgttgtgcttggatcactgtgtctcagtcatttgacaggaaagaaattctgaaacaaatgatcaggcaacttctgggtgctgattcattagacaaactcttgaaagaatttagtgagaagttgctcgtgcaagtccagcatctcgctgatcacttggttgaagggctaaaggagaaaaggtactttgttgtccttgatgacctatggaccatagatgcatggaattggattcatgatactgcttttccgaagattaacaacagaggtagtcgcataataataacaacgcgagatgctggcttagctggaaggtgtacctctgaatcacttatttaccaccttgaaccgttacatatagatgatgctatacacttgctactagcaaagacaaacataagacttgaagacatggaaaatgatgaggacttgggcagcatagttacaaaattggtaaaaaggtgtggttatttaccgctggctatactcacaataggaggcattcttgctactaagaagataatggaatggggaaaattttacagagaacttccttcagagcttgagagcaatccaagcctagaagccatgaggaggatggtgaccctaagctacaatcacttaccatctcatcttaaaccatgctttctttacctaagtattttccctgaagattttgaaattcaaagagggcgcctggtagatagatggatagcagagggttttgtcagagccacagatggggtgaacattgaggatgttggaaatagtcactttaatgagcttatcaacagaagtctgattcagccctcaaaagttagtacagatggagttgttaagagatgtcgaatccatgatatcatgcgtgatatcatagtttcaatttctagagaggaaaattttgtgctgttgactagggagaagatcactgttgtagcggaggagagcatccgccatctagcatttcatgggagcaaatgctcaaagatatgcttggagtggaaccatctgcgctcagtaactttgtttggtgacagacctgcggggcgaacacctgcactttgttcaccacaatttaggatgctgagagtgttggatctggaagatgcaaaattcaaattcacacaaaatgatatcaggaatatagggttgttgcgccacatgaaatatttgaattttgcaagagcctcaactatttatacacttccaaggtccataggaaaattgcagtgcttgcaaattttgaacatgagggaggcaaatatctcagcactaacaactgaggtgactaaactccagaatctccgtagcctccgatgcagcaggaggtcaggttctggttactttagcataatagataatcccaaggaatgcttgatgatcaccatgtgcttaccgatggttttctcaacttcaataaatttcagtgaccgtgtgaagttaattcctgagatatgcatgtcatgttctacccgttggtctgatacaaagggtgtgagggtgccaagaggaattgacaacctaaaagagttacagattctagaagtcgtggacatcaacagaactagtaggaaggcgattgaagagctgggggagctaattcagttaagaaaattaagcgtgacaacaaaaggcgccacaaataagaagtatcagatattttgtgcagcgattgagaagctctcttctctgcaatctctccgtgtggatgctgagggattctcagatactggaacacttgagtggctcaattcgattgcatgtcctcctccattcttgaagacactcaagttgaatggatctcttgcagatacaccaaactggtttgggaaccttaagcagctggtgaagatgtgcttatccagatgtgtgctacaagatggtaaaactatggagatacttggggcactgcccaaccttatggttcttcgtctttatcgcaacgcatatgctgacgagaaaatggcattcagaaggggaacattcccaaatctcaggtgtcttgatatttacttgctgaagcaacttagagagataagatttgaggagggcacctcgccaacaatggaaagtatagaaatttatggttgcaggttggaatcagggattattggtatcaagcaccttccaagacttaagattatttcgcttgaatatgatggtaaagtcgcgaagcttgatgtgctgcaagaggaagtgaatacacaccccaatcatactgaactgcaaatggcagaggatcgaagtcatcatgacctaggaggtatattatataaggatcaaatatagcatatgaacaatgaaaatcgcgtactgagcttgccattgacagatcatggtcctgaccaggccgttgcatctgatggcgatgatgcccatgacaatccagtgctgcgctatcaaacatcatgttagacatcaagagttcagtcttgcacttgacttgagaacctacccctgttatgtaattcttacctgacatattcatctgaaattcatctgttctgccgtcttgttttttctccctctctccagattgtgaccatacattttcttttactacatctttgcaccccaaagcgaagatacgtgcgtgctgatgtgatgcttctagcttgtaattggtgatttctcctctcttctccagctcagtttgccgcctgctgatgctatctctaatatcgaattctatgcggattcccgtgcaaatagcacctccacgtggcgcggccagattcacggattcgctcccacgcacagccgtccgattgcatttcaacggtgagagatcgttgtggtggagcctgttgtaatatacgagaatcgaggggttttcttacaaaagcactcaatccccccatcatccgtctcctctccagcgtcaccgccgcctgattcagcacatccgccgcgtcaccgccgctgaccttcgccctgacctcctccccgctgcaccgaagcctctctatcccctgcgcctccacatgtcgcggctacgcgggcatctcgttggatctatcatcggcggcgaggaggcgtggcgatggtgccgaggttcatcggccggtggaggaacatcgtcgacttgaggaccgtgcccaggttccgcatggtccccaactactgccaccccgtgggagccgccgaggggggcaacaactatggctttcagatgatagtttgatcatgcacacaatgcgttcgacgaaatgtccatgggcattcatgggtgtggctggcgccctgatgatgctcaatcctaccttcctcgttggattactttggtgttggtcgaagcattggccgaccaaggaatgtgctgccaggagttacaatggtggactcacgtcttagagggcagttcccttctgactcatatggtgcgatgacataccgtggcgtgccgtgaaaacctgagattggcaaatggcttactaagtgtgatgccggcgtgtctttgtcactgaccaattcggattgcttgagctttccctggattaatttggttgttcttctgaaacggtttataggtgcaaaggggtgcgagcaagactacaatggacaaggtgtgtgcaactatgagctgggataatggagatgctttcatggatatgttggtaagtactactttgtcagctcaattaatgtgttaatcttgttcacacttgattaatgaaattgcgatttcacatggaatttaacaaggttgggttttaatcacactccattttttagcaccatccaaaaaatctttaagattgttttagtaatttatctactccgtacataatatcaagataaataggcatagtttatgaacctttgaattctgcacctgttccatactaagaagcaaataagaaagaaacaaataaatactccagttggttagtgtgtttgctcatatatgccattcttaagaaaaacatcagtttatatatttatttatgtcctatttagtttgctgtctatcaactcttcaaaaattatggataaactgtgctacttcaattctttgcttttgatcaaatgacagcccttgctctttccaacaactctattgacaaagatgttaaacatattttgcccaacactacaacttttgctccttagttctggaaatgatattgtatactctataaaccaagctaattcttcatttgacttatggatataccatcatagaaatagtataaggagacagttgcatattattactacacttattttttgaatatatatttcatcattgtacatatatatatttagacagctcaattatatatctttctgacaattgctaacacaatatttattataacaaagtttgcttggaaataacataataaaggatataatgaggcaataagaactgtgcgtggagggccggaagatgatgtctgtcatggccggcaagatggacgagctctggatagggcgtatggagtggtgcgtgctggaatgtaacaagaacgccatcaacttctacgagggaatgggcgagccacgcaggtgtgctcccgtaatggcacatatacagactcactggcgctgcacactggaaagtacaatgggactaagagaagggcaatggggcgaaatagatcggtggttgattcttagttgtccagtctcaagtaataaagaaaccggagatgtgtgatttagggtattggataagttcgtgatgtcttagcttgatgttctttcaaatgtagtacacaatctttgcagaaggttaagggaaaataaacaaattcttaaattataggaaaaggcaaaaaaagataattcaggagagtcacaagccccattgcataacaagcccaaaactgaatgttggagacactctagatcaaaacgttagcaagcggaaatggtatactgttatacctaattttataaaattattttatctctaacctttttacatttcctttttaccttgttgcaattactgacaatcacccaattactggactttgcagatgacatcgaaaaaatcctaggaaacactaacaagcatccattcaggacaatacattttttttcatgctagcaatggtgttgtttttttcttgcacaattttcatctcaagtcccaacgactatttatttaattcgttattaaggaaaaaattgatggatgctttatctagacctctatgtacactaatatcaaacaaatctaagctatagataaattagaattagattggcctaatatcaattgggcgtggcaaagacgcgccattatttctagtgcttatactattataatgtccaagttttgagtcgatctgatgtcctctgaagtgatcatgtcgaggtaaagtggtcagtccatgcacagtattcttctgtgatgtcttgcagtttctgcttccaactgatccgtgacgtgatgtccaaatatttgtgttaatatatgtcagaatgtcacatataccaaattcctagtcgcagttaaacggcaagaaattatgcagtttggtgcactattcttctgaatctagcgtacacctcatttcaaggagtgacacggcttgccaggtgacggatgacttggtggcggacgcaggatttttgagatgggtattcgagacatacataggaaaaagaatatatgtgacatcgcaagctatatattacattgggaaaaaaattataaatctcattcagcagaggtttctttgaaggacggcagcaacggaggaatctgtactcatcaggttacaagaaggaaataatccagtgttaacttcagagagaagaaaaggtaggtaaagcacactcgttctttgtgctaattatgctggtttattcatatttatttctcaaagttaaaggtacttcatcttgagcgacgcaagtacaacaaagcaaggtgttttgtattggaaagtacagatgtacagctagcaatgcaaaagagaaggcagaggaaactcagaactcacaagggtacgtaggtccactataaatccctcgaccagtcgacctgcctctgctgttgttgacaggagctaccggcttactctgggtttattcttacaaattttccagattagagaaggtccaagagagtgtgatcgggtaagtgaaacctgatttttttttttcaaaagtttctatttgcatctcttgaacatttttaatcgtatgtctgtcagtttggtgtatatccacattgtttataatctgttgaaatggcagactaagtactggcttcgatacactctaatcaagcaagtttaaaccaaaaaaaatacaattaagagaggatgctggtgatggcgttgttttgagtttggagagggtcattatggaggatgccatcaggaagcccaacaccgacggtgtgagtaacctttgggtttagtggaaatccggcaaaatcttgcaaggtgattagtgagtgaagccattttcagttggtgcttttctttgattatctgatttttaacagattttcttttgttcagcataagatagcagatgacatgaactgttggtatgggtcatgggggaaacagtgctcagcattgcgtcgtccaactgccacaaatgacatgagcttgctgctgggagtgcaggagacatggtaagcatctagctcccacataataagattttccttttcataactgtgcctagatttattggaaccatgcacagttgttatgggtcattggagattgggagaactgttattgagaggcagaaggtgtggaggttcttgttgagtatataagcacgtgtaggcacgtttaatttattcgataaataaatcacgacattgaacgtgtatactagcattatcgcatcgtactatttacacatgtaaactaggcatgaaatcatgaacatatcgaatatgcaaaacatggtgaatgcgtactgagggttctagctgcacagtacgaaaacacatggtactggacattgacgacgacgcgtagcacttgagcgaaaaggaagatgagccgtgatgaacgaatagcaagcagtcatgcaaagcgcttcccaaaaaccttattgtcaccttctcctggtgcaggacgttgaatgcgaaggttccggagagtagctctcccgatcaccgatgcacgccgacgatcggggcagagtagactacgagcgacgatgtagtatagaggagacaaatcttagattgatttcacatatgttgcgaggaggccgtagcttgatttatgtagagatatcaagacgcgtgattaggacgcctgcatgatctccataccatgtaaccgaaccggataagtcctgtgtaacttatccagactccacgccgatccacgcaccggattaatcggtgtgtttccaaaaactaggtttccaaaaacgaaaccgaatttctgcagcaacacaaaactgcaaaaggaagccgcatctacgcaagggtgaggaaccaattttgcagacattcacgctcatgccatgtacacaagccgtttttataacaggaatcattcgtgttaattattgtgccattgcccttgatcaagtagtctggtactcacgattacattgctggaatatccagtgtacatacatatagcatcttgcgtagattattacatcatagagcccgtaggcatataatagaacatacgaccaaacggtccagaatacattcaaagacagtgattagataaggcaccggagcttaggtagtgacaacgatcgtaactaagacctattgggcgccatcatcttcagcaacattcagatagaacttataagggtcctcagtgacttcattatccacctctgattgattgtaaatagtaagggtgagtaccaactgtactcaacaagccaacacaacaataatgcacatgataagggaattcagatggggtggctaagggatattttgcataaagccaattttgtaattcttttctcaagcctaggacctaccatagacttatcaagttttaatccattatttattaactagtgacgtttctgtcctaccatccattgtgatcccaaggatagcttcccgccactgtattgtcatggttttctagggtctaccccttctcgccacttgagaagttgatccactagtacaaaatcatcacgcatcatcccacccatcaatttaagaatttgagtctagccaagtgtaatacatgtcccggtgctcattatccgcaagcatggctatttgaatagattggtttactcacactgcagtggatgtacgctttaacccgcacctcttgacttgcccaacaaatgagccgaagtcccaacgaatgaggtgcatcacgacaaagccttttaataacctcactttggtagtacccgttccatgaacttaagccctcatgcactttaggcgtacacctgtttctagcagagagaagagttctggtgttcccgaaagggataaactcacacatgcattgaattatatttaagtcaagttttgtttaggttacccactgatggttatccatctgtaggctccccgcctattcacacacctacatgaatccaccacatcttgggaattggctctgctcggctatttactctgctagatccatactcaatgcgtgaagtacggttgtagtggggttgtttcaaaatcagtctttagaacaacaatattaatctgagaaggaaattcatatggattcgcaactttttaccaaatgtcataactgttaaacataatataagattgcatctttttgcaccaagaaggaaaatcaactctatcataaacatgtggcttggtcgaaaatctttcagccatggcaaaaactcaagatcagtaaagatcaaaaagagcaaacgtgactctgataccacttgtaggatcgaacacaaacaaccaaacctaccagggggtgaatggtagacatcggtcacactgctagattgccgtcggtttgatcgtggtaccctcgctggtctgactgccgccttgctactggttggaccgcctccctgtgccggttagattgctcgtcagatgctgttctgaccgtcgggatctagaaaaacacgtctcgacgaaactcttgaaattagatcaactttattgcatcaatatgtgtttacaaagtgtatctaaagcacccctctcccacctcacaaactagaattgaagcctcgaagcaaaccgtaacttttctttcccaaaatgtagatatacttctagtctcaacccctcttttatttatacccaaaacatggctgcccctagccacaaatccaactagaactaggactcctaatcacataggaaacctaccaaaccagggaaacaacattcccgtacaacctcacgctccaatccttccaaacttggactccaatcaaaatttaactctttttttcatacgcacacaacaccacgtgtatgccatatgaaaaccttcacctccacatgcactgttctctagcctaagcatcccgtatgatatccttgagcgtctagacctgaacttgtcccaaagcctagccccgatccatcaccggctatactcccatggccttaatcaacacctgcacatgaacaacaaagaaagttcatatccgagtacaagttcacaccaacttaacttacatcagcacacaatagtatcacatacgaatagaaaccatctagaagtcataaacatgaaacaatctagaaattccacgaaaacaacccgaaactgactcaaaactggagtatgccgatctgacagttgggctacccggtcaaatcctccggacacctccggtctgaccggcgcccactgcccggtctgaccggccacctagtgccaacaacatccgaaaacactattcaccgtactttcaacaaactcttacctttgataatcgttcatcaccacgattaaatcattacatgattttacaccttggtgacatgaaaatgattcatccaaaagtcaccatggatgggaggaggaagaccacatcttgggctatattgggctctattttagtaggaatggattctaatctctcgaaggggtgccacattattattgcatgctatctaaatagttacgaaccaatttgaaaagacaattgaaaatatagattaatatgtaatatatcacttcacagacatgcaagtttaaattcaacctctgaacgttctaacaaaaagaacaaattaaactctattcacagttgtctttctttctttcgctataatttgcaaagattgaatttgaacttacatgtttgtgatgtgatatatcacatattaatctatcttgtcaattgtttctaatttttgcataactatttagatgacatggaaaaaacgaggggatatttccttgagagattaaaatagtttccctactttagctgattgttttcaagggccttagtcagtttgctggcagcagagcattgttaccagagcatatgtctgatccaatttgcatgattatagtaaactgtttttttaattttagcaatatctgcatagataacagccaaagtaaacaaaaaatgggcttcagaatgcttgtactttagtattaatttagaattatatttagattggaacattgctatgtgcattttcttgtgtagttttgtgacttccatgatcacacctctctattttcatcttggatttgccacttgcccattgtaatgaggattcattgttttcatttcatacgtatagtatgataatggagtttctgttccattttattcaggtaaatgaatgagagtaccttatcgcaagaaaacctaaagaactccaccatcagtgatcttatatattattgatccaagaattgatttggaggaaggagatgactgagacggtggtaagcatggccatgtcgctggtcgggagtgccatcagagtggcttcttctgcttccagccaagagatgagcatgctgattggtgtgcaaaacgagatttggtacatactctggatgcaacttcctcatccttcatcaagttcttgattctcttgtgacaaccacttttttccgcaatagcttcaactagaaatgcgattgcttagtgattgacaggtaatatgtgatggcaatctttgaactgtgattgacaggttcataaaagatgagctgaagacaatgcaggcattcctgagggcggctgaagtaaggaaagatcaagatgaactcgtgaaggtgtgggctgagcaagtgagagatctagcctacgacattgaagattgtcttcaggagtttgctgtccatgttgggcatcaaagcctatcaatgcagctgattaagctctgccaccgacaccgaatcgctgtccagattcggaacctgaaacagagggttgaagaagtgagcaatagaaacatgcgttacaatctaatcaagtctgttccatctagcagtaccgatgactcccaaagcaacatggaattgattcggtatcagactgctcattatgttaatgaatcagagcttgttgggtttgacgttcccaaaaaggagatcctggagttggtatcaagcagtgaaaatgctgaagcccaaacaatttggattgtcggagaaggtggtcttgggaagactactcttgcaaagaaggtttatgaaagctcagatgttagtagcaagttcccgtgtcgtgcttggatcactgtgtcacgatcatttaatgtcaaggatttactgaagaacatgatcaagcaattactaggggaagactcactcaagaaactcttggatgaacacaaagaggtgattgtagaaaagcacaaccttacagaccacctgaacaaagggcttagagggagaaggtattttcttgttcttgatgacttgtggaccacacaagcatgggattgcattaaacctacttcctggggaaataacatggaaggtagccgagttgtagtaacaacaagaaacaaaaacctagcaggtggtagttccacctcgccagtctactgccttaaaactctagagaaagaagatgctaccaaattgttgttaagaaagacaaaaagaagtctgcatgacatagaaaaagatcaaatgaaagaaatatttgaaaagatactaaagaaatgtggaggtctaccgctagctataatcacaataggtgcagtccttgaagggaaagatataaaagagtgggaaattttgtatgctcaacttccatcagaacttgaaagcaatccaatcgctgaaccaatgaagaaggtggtaacccttagttacaattacttgccatctcatcttaagccttgctttctgtacctttgcatctttcctgaggattttgatatccaaaggaagcgcctagtacatagatggattgcagaggggtttattagagctaggggtggagtgggaattgtggatgtggcacaaaagtattttgatgagttgatcaaccgaagtatgattcaagcatctagagtggatatagagggaaatattaagagctgccgagtccatgatatcatgcgtgatgtcatgatatcaatatctagggaagaaaattttgtatacttgatgggtgatgatggaactagtgtagtggaggaaaatattcgccatttagtgcaccatgatactagcaagtgttcaaatataggcatggattggagccatgtacggtcattaactttgtttggcaatgagagacccaaagggctatctccttcattttgttttccccaattgaagatgctaagagtgctggatcttcaagatgtcaaatttgggatgacacaaaaagatatcggaaaaatagggttgctgcgtcacttgaaatatgtgaatattggagggcattcaagtatttatgcacttcctaggtgtataggaaaattaaaagacttgtgcactttggacataactgacagttacattacagaactaccaactgagattagtaaattgcagagtctatgtattctccgttgtagaggaagaccaaactcgggggattttaatctaaatgatcctaaggattgtctaattgccttctcatgtttgcctctgcttatggctgcaaccgattctgatgaacgtaataaaataattgctgagctacacgtgggttgttcaagtcaatggtctcctaatggtggtacatatggtgtgagggtacctagaggaatcaagaatttgaaaaggctgcaggtgctagagacagtggatatcaatcgaaccagcagtaagtcagttgaagagttgggagagcttatccagctaagaaaactaagtgtagtaacacaagggtccaccaaggaaaaatgcaaaatactctgtacagccattcagaagctaacttccctcaaaactctctatctgaatgctcatggacccttggatactggaacacttgaatggctacattctatttcccatcttccttccctgaggatcatcagattgatcggatacatgaaagagatgcccaactggtttagggagctcagacagctggtgaagattcacttgcagaatagccaactagaggaggataaaacaatggagatactcggagaactgcctaatctcatgctccttttccttagttggcgagcgtatgctggggggaagctggtgttcagggagggaacattctaaaatctcagggtgctaatcattcgcaatcagaagcaactgaaagaggtgagatttgaagagggaacctcaccctggatggaaaggattgatatcagagaatgcagattgacatcagggattgctggtatcaaacaccttccgaggcttaaggagatttcacttgaatacagtgctaaagtggtgaggctaggtcagctagaggttgaaatgggcacacaccccaatcgacccatgttgcgcctgtttggggagcgaagccgtcatgacctggggaacacccaagtaactgtcgatgaacaacaactgcaggtcagtccatttatttatggtgtgcatgcaactacttgtttttctttctcttctttctcttgcttactattttcctccttccttgtttgtctctagccaaaccaaacagttagcgaagaatgcttgatggatgccgatcttgcttctagatgatgatgatcagcttcctgttgcaagctctcctggtgatcgttcttcttcagttcttgacctaccaccaatgctggctagcttcctgtagtgctgatctcggagtcccctgctgaattttgcacctccggcgccgagaggccagttcactgtacatcccccctgagacacaatgtcggtatttgtttgattggatatgatttgattaagtacgatgatttttggttctgaaaatttgtatcatgattggttggagcgtgtgattaggatgcttatatttgcagcagaggtcgatcgtattttgttttgctgtgcacacaaaatgttcgtcccaaatgtcttgtcagatattttgtagcttttgattatcaagtactccgtatttatgtttcgtaatatgaaacaccattgtcaggctctgtgcacgaattgatgcaatgtaatgatctaagttgcggttccatgtgttcatgctcttcggatcaatttagtgtactgtttctatctaacgtaccttctttaccatattttcttaaaaaggaactaatatatcttaccaatcgtttctgatgtaatatttctccatccattgtcatactgttgttgtagacatcaaaatagtacttttttttgtctctcaaaaatttatctgtaccgacttgtatctaatagatcattttgtttctgtgcaatttgaaccagcccccttttgccaaaccattatggtgctggtttgcgaatgttcagttggagagtagcttgaccaaatgggcattcagcttaattgcaatggattttagtgtaaaatcacatgatgattaatctttgtgatgaacaaattgtcaaaggtgagtgttttggaaaatacgatgaatagtgttttctgttgtgaacaatgcaggtcgacactggattgccggtcagaccaggtggtgcacggcagtcaaaccggaggtatccgggtggtctgaccgggtagcccgatagtcagaccggtagactcggggggtggggggggggtcgtttcgagttttttcatggaatttatagattaattgcttcatgtttatgagttgtagatggtttctattcatatgtgacattgtgtgctgatgtgaatcaagttggtgtgaacttgtattcggataagaagtttctttgttgttaatgtgcagattttgctcgaggccatgggagtatatccagtgatggaccgggactaggcttgggagaagttaaagtccagacagtcaagaatatcacgcgggatgctcatgctagagaacaaatgcatgtgaaggtgaacgttttcatatgcatacacgtggtgttgtgtgcgtatggaaagatgagtcaaatttggattagaatccaagtttgaaaaggttggagtgtggagttgtatggggatgttatttccctgatttggtagctttcttatgtgattaggagtcctagttttaattggatttgtggctagggacaaccatgctgtgagtataaatagatgatgggttgaagctactatatatctactttttgggagagaaaagttaggatttgttttgaggcttcaattctagtttgtgagttgagagaggagtgctttagatgcactttgtaaacgcatattgatgtaataaagttgatatattttcaagaggttcatcgatacgtgtttttctggaccccaacggtttgaccggagttcattgtgcggtctaaccagcaggcggtcacaccgacggcagggaggcggtccaactagtggtaagtgggtagttagaccgatggtgtgcaggcggtcagaccggtggaagtattgtagtcagaccggatgtgatctagcggtgtgatcgacgcaacttcatcaagtttgagagtaatcttttaattccccaaaaaaatttggatttggtgttcctaccattcaaccccctctggtagggttggttgcttgtgttcgatcttacatttacttgctcagagtaaaaccacagatcatattcattctcactaatctctagcaagggatgggagcaaaaataaaaacaatccaatggtggtcgacgtaacaagacagtttcaaccactaggtatgaaaacagagcggatagttgttgatggttgttaagtgtaaactttaaccgtcaacttctgcataaaaaatatcaaatatactatccaaacttagtggtaggagtttactactaaccacttccactagttttggtaaatatatgtatgcaggttaattaaggagaaaatacacccgacatgtgacgaaatgaacctccggccaatcatgtgcgagtacaagggttatattggagggcccacaagtcagtcgaaaccaacttgaaatgggaccaaactatcctaaccgccataatggcccactagtcagtctcatggccaaagaagaggccagtaggtgggtcccagggttcgaccgaaccgcctttagcgcctctccatcccggccttccacgtggacgtaccagattgtctgccaatgacggtataggggtgttaacaacattcccaactgcctaatgactatatatgtttttcttttgcctaatgactagtttttttgttttattaagctatgctatactacttacggttataacggttatagatgaatatagttcttgtttattatagctcttctgaaaagtgcagtgtacaactagttgtattattgttaactgataaaacatagaaatgttgatttaatgagctacatctcttcttcgttgatatgagaagcttggtttgagttgtttttatattctaataaatatttgttactagaatcgctccattttcgtatttgaaaatatttgattatgttttttatgtggggtttctgattccaattaaaaaaaatgaaaataaaaatggtacgttcgttttcgagccgtttcatccctacttgcacatgcatgctgggaacaatggatctcactaatcaagtagcacgcagttctttatattttgttgattatggcttgtgaagcatagattcaccgaatcaattgggatgttaaaatagtcacatacatacctaagcttgcatgacgttatgtagatagagatggcgaatataatgcgctggaaagtcccaagtgaggatgcaaaacatcttatagtgggtagtggagccctgcaaggaccctaatatctaaagcgcacctaaactgatatggacatccattatagttaaagttagggggaatatgattctcttcctgtgcacctaaaccgtaatatgcagtgaaacgaacgctatgatacgatgataagcttaattcctctctctgctcagactgttcagtgcaaaagctaccaacgagagcttgtctccttgtgcggtcgtgagcttgcttgtgctaagcttgaagggagagtcgaacgaatccatggcggagacggtgctgagcatggcgaggtcgctggtgggcagtgccatcagcaaggccgcctctgccgctgccaatgagacgagcctcctgctcggcgtcgagaaggacatctggtacgtactgcactgctctcgtttatcctagcaagttcttaggctcttaatctcgaaattgaggaacaccatgaaacactaaaagagagctcgaagactaggaaagaaaactagaagactaagctttgaaagtcttctaaatccaagcatctcgacattgatcatccttgtgcaacatcatcccttcctattgcttcaccagaatcggtgtcccttgtggagatctctgtcgtagcgtcaaggggagaatccgagaagcagaactagtccgcgctgccttcgctacgccatctccgccatagaggatctcatccacgaaacatccaccatccaaacgggaaactgttttaaacactcgggtggatattcacccgtttcttgcatgtcatctaaatggttatgaaaaattttcaaaaaaaaaacatgataggttaatatataatatatcatctcacaaatatgcaagttcaaattcaacttttataagttgtaagtataacaggacgttcatctcacaaatatgcaagtttaaatttaacttttacaagttgtaagtgtaacagtacgtccatcggatagattaatatccatctccccatccaaacccgttgttgcaccatctgtcgaatccggctgtggacgctcggaggcaagagctagctcacccgtcccacacacacacccaacgacgtcacaagcgcctccgaacaacgccaactgataacttggcagctcctacgtgccgacgtcgcggtacttgccggcgctcctagcgcacgcaccgtcgaaccacaccgtcaccgaccaactacccaccgccgccgacttctgcctcatctgccatcgtcgccctagcccaagttatcatcgtggcaattgccgaggctcctaagtgtgccacggccgaggcaaagttctaactgaatcagagtatcagacagccaccaccgacgctacttctgcttcatctgccatcgccgtactagttcaagttgtcgctgtggcaatcatgggccctcctagcgtgccacacaaccggacagccacgacatcccccatcactgttgttattgccgcgccctgacccctatcgtcgtcgctcttagcgcgtcgtcgagccgaccagccactgtcgtgcagatgaaaaaaaaacacatattggcctgagagatctgcttagttccagtgcaggtccaacatgctgtgagatgcgggcgtgccagtcagtttgatcttgcaactgacaagatatataaatagcagataaaacagcctatcgactaacaagccgatggagtaattccagccgatagccgatattagccgatgccgattctagccgatgtcgatagggttttgaactatcggctatatgtccaatgtaggtaatgatataaagacaattggctgatgataataaaatataaaaatataatccaatagaaaccaatcggctaataataagtattgatccgatagttaaagcatacatcggctaaaagtccgatgtcataaaatccaatcgatttagataaacagtgaaacctttgttgcaatcggctaaatccaacttgtatgtaatcttcgtaagccgatgaacgtccagataacttatcggctagcacctcgataaaacactagcatgaacctatcggcttaacaagatttatattatcaacaacaatctagtaggtcggacctaaccgatgcaacacgtattagatatgataatctaatacttgatgagccaataaatctgtctaatgtgatggatataacaaatctatttataaaagcattgcgattgtagagatatatcggctaagacagaatatcagacctaactaaaccgatgcgtctctaaacacaatgcaattaattagagatataattgagatatcagctaggcaaatatatcaatcaaactagagcgatccaagagatcggagcaatgcagccttgaacaacaccaatgtagccgatggattcaccagggccgacggaacgtaggacttaccccttccctgaagatcgggctgaaccaatgcagtcccacgtcaggtgccaaattccgccggttgataagtaaaaccttagaaaagaggatgacgatgcgccgagagtagtattgatcgagagataaattgcaatgaccctggatgtacatatttgtacccatgggtagatattagttcttgtaggacaagaaagaaactttcctaaagataaaatgaaaacataaagtctttattggatactaaacacactttcctaaagataaaaggaaactaaaccctgcctaattaatagataaactgccatgtcgtatcctccttgaactcggactcttttagataagcttcctttaactaatctttacccgaatccatcaagaatacaaatgttggcattgatagttttcatcggtcaattataggactttgaagccgatactgactctaagccgatgactactttgggcttaccaaattttgttgttaatatgtcgcgaccaccatcaccggccagccaccctgatcattgttgttgactcagcattcgccaggctgagcagtccacatacatgccgccatctccatggcagtgtcgttgccgcccctttctcctagagccgccgcagcgctcttcgacacacctactgcatcgtcgagcagtcgtgctaccacctcctccatcgaccatagccgcctcttctgctgcaccggatccacccacaccaaccaccagatacagtcaagccctcattcctggatcccatatccatccatgccactactgtgctgcccagtccaaggaatggagcgaaggaggaagccccgccgctgccctcccggcggccacatgcactccagtgccttgctccgacggcagcgaggttggaaaatgggtggcagcggctagggtttatctggggagaaggaaaaggagagggagggggggggagggtccacttccagcttaattagcctagatcttattgacaaatcagttgctgggtgcacaaacatgttattttttttgcatgaccaatcttgaacacttaggtatgttagttgagtggacactggtctatctgaaacatctcttcacatggaggctgcgaatgagttttctttttgagagaccaaagtttcgttgtatgttaagtgataaagccttggtaagaaatgctaccacaaacgaactaataactccaaacgtaaagtggaggaacccgtatgggtgactcgagtggcgacaaactctagcacctccacctccttggacgggctgcggcggtgctttcggcatcccagtcttcttggaggcatcatctagaattaaggtcttgttattgcttagcatgccttagggcacgtccagtgtttagttcgactaaaacttccatgaaagccaaacaaaagttctgtttgaccaccacagtgtaaaaatcgattgtgggacccatgcaaaaaaatcacaatctcagctgcctatgctctcctcctggacctgatagccgtgcacaacaaatatttttttaaactggatgtgttcggcttctctttaaagatcgttttttcctctgacacttaccaaccggctttcacagtgtggtcagttctttttttttttacgcaaagtttgattttagtcagacacgggaggatctgttaagcaggcttggaaatttcggacccctccaatacaatattattttagccaaaatttctaattttttaatttttcatgaattttggtaatatttgttctaatttaactaaattttgttcaaaatttcggtctatcagtgacctccgatcaaatcagttaaaccgagaaaataaaccatgctcttaagagagtttggtatggttcaatatcaaaacttatagtcttgcaattttttctaccctttatctttttccctgactatttagtatggatcgtttaaaaaaaagaaagcccattggtgaccaagggcttgtttgattcaagaccatccctagccttaccaaccttttggcaatggcaaaaattggttgttgccaaaaatattggcacaaattggctaagcctatgattggtttctaccaaagttgaattttggcattcaatcaagccaaataatttggcaataacattttcttatctatggatataacatatggcaaatattttggcattaccattttctttttgccaaacatgttattccttttgaatgaccaatcttgacaccttatgtatgttagtagtggaatcgacactattctatctaaaacatctctttacatagaggccgctaataatttttctttgagataaccaaattttccttacaagttaagcaacaaagcccattggtaagatatgctacgacaaatgaactaataactccaaacgtaaagcggaggatcccgcatttcccacgtgggtgactcgagcggtgacaaaccctagtacctccacccccttgggtgggttgtggtggcactttcggcaccgtattttccttggacggatcatttagaaagtcctattattgcctagtatgccttgacagtttaggcaacactcttggatggtggtgtcctttgccctggtgatctagtagcccatggatgtttagttatttggacatggtgttggatggtgcgctcgtgggcctgttgtaggtctggtgccaaccagtcatgcttagaaatagccggataggtgcacagtgctagttctttacttggtggtttgtgcagcgctatcgacatgtggtggtgtgctttttctttgtccggataataatctcatagggctatactcttgttattttgctgctatattattatgataacttggtatggttcgttttttctttttttggaaaaacacctagttgatcaagggcttgtttggttcaagtgcattcctaatcttaccttttcttttttttttcaatggcaagaattgttcattgcaaaaaaaaaaagagataaaaattggctaggcttacgttttggttcttaccaaagttgtactttgagaccaaatatatggcaaaattttggcataaccttttttttttttgcttggttgagcttggtacaaaccaatcagtcacaaaatagactgtcatgaatcacgcctactaaattcctttgaaccgaactagaatatatttgctcttaaaagatttcttgatttcaattggtaccatttactagtagaaacttaaatttaaattttaaaaacaaaatcataatattgttgttatggaaattttagtcattttagtacttttgtaatatatgagttgggttatacttgagatatcctaaattgctttaagatgaacaattgctaggtatatcaaagatgagctaaaaacaatgcaggcattccttagagctgctgaagttatgaaaaagaaagatgaactattaaaggtttgggcagagcaaatacgtgacctgtcgtatgacattgaagattcccttgatgaatttaaagtccatattgaaagccaaaccctatttcgtcagttggtgaaacttagagagcgccaccggatcgctatccgtatccacaacctcaaatcaagagttgaagaagtgagtagcaggaacacacgctacaatttagtcgagcctatttcctccggcacagaggatgacatggattcctatgcagaagacattcgcaatcaatcagctcgaaatgtggatgaagctgagcttgttgggttttctgactccaagaaaaggctgcttgaaatgatcgataccaatgctaatgatggtccggccaaggtaatctgtgttgttgggatgggtggtttaggcaagacagctctttcgaggaagatctttgaaagcgaagaagacattaggaagaacttcccttgcattgcttggattacagtgtcacaatcatttcacaggattgagctacttaaagatatgatacgccaacttcttggccccagttctctggatcaactcttgcaagaattgcaagggaaggtggtggtgcaagtacatcatctttctgagtacctgatagaagagctcaaggagaagaggtactttgttattctagatgatctatggattttacatgattggaattggataaatgaaattgcatttcctaagaacaataagaagggcagtcgaatagtaataaccactcggaatgttgatctagcggagaagtgtgccacagcctcactggtgtaccaccttgatttcttgcagatgaacgatgccataacattgctactgagaaaaacaaataaaaatcatgaagacatggaatcaaataaaaatatgcaaaagatggttgaacgaattgtaaataaatgtggtcgtctaccattagcaatacttacaataggagctgtgcttgcaactaaacatgtgtcagaatgggagaaattctatgaacaacttccttcagaactagaaataaacccaagcctggaagctttgaggagaatggtgaccctaggttacaaccacctaccatcccatctgaaaccatgctttttgtatctaagtatctttcctgaggattttgaaatcaaaaggaatcgtctagtaggtagatggatagcagaagggtttgttagaccaaaggttgggatgacgactaaggatgtcggagaaagttactttaatgagctaatcaaccgaagtatgattcaacgatcaagagtgggcatagcaggaaaaattaagacttgtcgaatccatgatatcatccgtgatatcacagtttcaatctcgagacaggaaaattttgtattattaccaatgggagatggctctgatttagttcaggaaaacactcgccacatagcattccatgggagtatgtcctgcaaaacaggattggattggagcattattcgatcattagctatttttggtgacagacccaagagtctagcacatgcagtttgtctagatcaattgaggatgttacgggtcttggatcttgaagatgtgacattcttaatcactcaaaaagatttcgaccgtattgcattgttgtgccacttgaaatacttgagtattggatattcgtcatccatatattcacttcccagatccattggtaaactacagggcctacaaactttgaacatgctgagaacatacattgcagcactaccaagtgagatcagtaaactccaatgtctgcatactcttcgttgtagtagaaagtttgtttatgacaactttagtctaaaccacccaatgaagtgcataactaacacaatatgcctgcctaaagtattcacacctttagttagtcgcgatgatcgtgcaaaacaaattgctgaattgcacatggccaccaaaagttgctggtctgaatcattcggtgtgaaggtacccaaaggaataggtaagttgcgagacttgcaggttctagagtatgtagatatcaggcggaccagtagtagagcaatcaaagagctggggcacttaagcaagttgaggaaattaggtgtgataacaaaaggctcgacaaaggaaaaatgtaagatactttatgcagccattgagaagctctcttccctccaatctctctatgtgaatgctgcgttattatcagatattgaaacacttgagtgcctagattctatttcatctcctcctcccctactgaggacactcgggttgaatggaagtcttgaagagatgcctaactggattgagcagctcactcacctgaagaagatctacttattgaggagcaaactaaaggaaggtaaaaccatgctgatacttggggcattgcccaacctcatggtcctttatctttattggaatgcttaccttggggagaagctagtattcaaaacgggagcattcccaaatcttagaacacttcgtatttacgaattggatcagctaagagagatgagatttgaggatggcagctcacccctgttggaaaagatagaaatctcttgctgcaggttggaatcagggattattggtatcattcaccttccaaggctcaaggagatttcacttgaatacaaaagtaaagtggctaggcttggtcagctggagggagaagtgaacacacacccaaatcgccccgtgctgcgaatggacagtgaccgaagggatcacgacctgggggctgaagccgaaggatcttctatagaagtgcaaacagcagatcctgttcctgatgccgaaggatcagtcactgtagcagtggaagcaacggatccccttcccgagcaggagggagagagctcgcagtcgcaggtgatcacgttgacgacgaacgataggtcagtcactccctacatggcagcttaattaacttgtttctaattctcttcttgttcagtattagccatcaggtgagggcgatgatttcaactcacttttcatctctctcgttttcttaacctgacagcgaagagataggcacagctcaagctggctgacgatctcctcccccatcagcgtcgtcatcagcgaacagatagggcagggcttccctgcttctgcgtgcacctcactgctctgactctgagggacatgatgatcaatgaggcttccagttcccaaatgtgcgcctaacacactcagtcattcccatccaaggtatgaattgatcgatctgatttatttcttgccgtgaggtgcaaatgtttgatgcttactttgatcagggtattctgtttcgggctgtgatatgcacaattactctcaattcgtgttttgatgctgagtttttatttctcttcttacccgtgcactcttcatttccatttcattcataacagaaaacaatttgattgcattgtggaagggaatatgagatcagaaatcagatggttagttgtggttttcttatttcgtttgctatgagcaccaatcgtttgctagaatgtctgaaagatcttgtgtacatatggtggactgaacaattgaacattacaagttatcatattttatattgttgctaaccgatcacatagtccagtagtccagtagtaagatttttgttaagtttattgttactgaatatattgtttggcctgcagttgttatttctctcaaaacaaaattatttggtagtctcaagtacaaaaagaaagacagatcagacaagttgttttactctactagtttcaaattgatcatctctgtttgttcttcattcattttctttctgtaagagagtttgctaggatggtgatgtggtcatgtggatgatcaaatggactacatcagacgcatcacactgctgcccaacctttacccactgtagacaaatggagtgcaggtcctaaaccaggccagaagtttgttcagtgttcttgttccaaaataaacattctggatggcaggttatttcattataacattcactcttatagctttcttagtcaaaactacaaataaggtctcctaaaaaatgcatcgacgttgatatctgtgttttctgccatgcagaatgacttgctctcaatggttgaagctgcaatcctccatgctcatttctggctgaacccaaattggtggcttggaggctggagctgcatgacattagagataacaatggccactttttgttgccatgggtgagggtacatggatcatgccgctgagcctctactccgagcaagcagaaaactggctggctgtagatcgagttcgccatcgccctcaactttgtcgatgcgatggtgatcatggatagatgtatgtcacatagcgcaaagcggagcctccaatgttcgcaaccgtactgtaaatgtgggagcggcgaatttctcaaggggacaaccatggaacagaggagatggagcaaagtagttgctcttgctatcatttcagagctcaggctgatctctagctcagataggagtacgaattcttatgtgtgtgtatttgatctatgatagtacgttttaagagttgagccagatattgcttgatcattacatgatgatatgtaaaaaatggtattgacaaggagacgcaggaacagggtggttcattgctctgtttaatcttaatcttggagagctaggatggaaaactgagttggtggttatatctctactacttttgatgttgtcccaaactaatttgtttctactcatgttcgtacctaaaaaaggaatattttagtgattacagaacttaattttctctttaatcagattatttatcagtgggatttttctttctctagttctgtatgaaaatactttttttatcgtcaatcctcctaaaattttgtgatatcagtattttttttgttttactggaatgggctgtttcggtgctgcttgcttggacttgctgattcctccctctttcctatttataaactcatttttcttcagttttttttcctgtattttggttttctttcctctacgtgactacacattttgaatcgaacatgctatgctctgtatatctgcttggaatacttattaaatgcataggccggccatttggaatgtgcacttaacagttgtttgaacacttccatggatttgtttcctcagttgtcggacggctatttaaacctgattaagaattccatgtgcagagacttgtactagcgtcgaagactttgcttcggtgactcggagtcaagtcaaggccggcacagcgcaagccaataagctacgacgacgacgacgacaaccatgccgcgctattgcttgtaaactttgtctacaaaagccagccgcgatgccatccatccactccttccttccccattgttgctcagccactcggcgtcggattttcctctaccacaccagctctaccaacctttcccgagattagagaggaagaggaggacgccgaagccgttgcatcaagcccaggaccaccgtcgaacacctgctgtgcgcaccgctcggcattccttcgcttcaccaccgtgtgtgcacagttcccaccagagtgtgcaaagcagcataggtaagtcaactccgattttctgctgttctttttttttttaagataaagcaggagctctgctattcaattaagcatggaagaatttttgggtattttgtgtatattctggccttgtttagttcccaaaaatttttacccaaaaacatcacatcgaatctttggacatatgcatggaccattaaatatagataaaaaaaataattacataggaggtttttttccggtccttgagggaaggcagtaccatatcctagccgttgattttgcatgatctaacggctggaaaacctcggtaccgcgtggtaccgcgtttctgtgagagtaggtaccgatcagtttttgaggtggaagggtatcattgtaatttcgcgtcacttatcccgatcaacctaaccgtggacgctgctccttcgagctcgtcgctgcggggtgtatgcgacggcgggcgtggcgtgacggcgatatgcgacggcgggatgcgacggcagatgcggcagtgaggaggcggcggcctcgatgcggcggcggccgcggcgcgatggcgggatgcgatggcgggcgacccaagccgagatgacgatgacaacaagcatgcgagattgatcgatgcggccgcggtgcgacggtgggatgcgacggcggcggccgcagtgcgacggcgggcgacccaagctgagtgcgacggcggccgcgtccctcgtcggctcgcgggacgcggcgggctcgagggggacggcggtggtggctggacgcgcggcggcaggggcggctggacgcttgatggtggcgggcggctggatgcgggacggcggcggctggatgcgcgactgcggtggccagctggatgcacgatggcggcggcggcctcgattggcgacgacgacggcgggctggatgggcgacagtggcggccggcgggatgcacgacggcggcggcggcgtcgattggcgacgacgacggtgggctggatgggcgacggcagcggccggcgggatgcatgacggcggcggcggcctcgattggcgtcgacggcgacgggctcgatgccggcacggatggcctcgattggcgtcgacggcgacggcctcgatgccacaacgaagagaggacatctgtttttgttagtccgatcatacccctactaaatcaatggtcagattagattggtacctcatggtacctcctcaaggataggaaagatgctcttgctatgaagaacctccgagctcagtcaatgtccggaaaataacttggataactacaagtggaagccatttttggttggttttctgcatgatctgtttgtccctgttttattatatcctgtaaacttttcattgcgcagtttgattaatacatgtctatttgatattgcagataaatacagttgtagcctgaaagatagtacttacaatctattgcttgaaagtctaaagaaagttgttcagagaaggaagatcagatggcggatacagtactcagcattgcaaagtccctggtgggaagtgctgtaagcaaggttgcttcggttgccgcagacaagatgatcatgctgctgggagtgcagaaggagatatggtgagcatctgacttgcagcctaattaattttattttcagttgcattagatttattgggaccacacttatgcagagtggtatggtactcagtttatttttaatgatttatttatatttttataccatgttctggaggaatgcatatgcagttttttctataagtatattatttgcaacatcttggggagataaatgtagaggaaagtgaaagtagaatgcactggaagtccctctatatacccaaacacaagtggacatagttttctcacggcaaccatgttcaatgaaggaatacaaacgaggcagctattaaggacctggtgataatctaatttcgacagaaacatggtttccttcgagtgtaaacaatgtagcatggcatgcttaagtcaaatgcatatacaatttgacaaggaactattagtttcagtgctgtgtaattttgctttttgtaggatcgaacagaaataactaagccaaccagagaagggagggggggggggtgaatagttgtagtaccaaaaaccaaaatttttagcggaattaaaagttaccctcgaatcgataaattccgatctgaccaaagtagatacgccggtctgaccgcttggatcccgtcggtctgactggagtatatcgtccggtctaaccgcccgaagaagctgaagtcgccgccggtctgaccgccgtgtacccgctggtctgaccgccgcgatgtcgccgatttgaccgccggtgtcccaccggttagaccgccgaactcaagtaaatacaaattgaagatctctcaaagtagatgacaactttattgcttctctctatgtttacaaagtgcaacaacagcactcctcacgaaaatctcgactaaactcgaaaccctaactattctctcaactcaatactctctaaagcgataccgggaggccacaccctccctctctatttatacatagggtaggcagcctaaagccacaaatcaaactcatgcaagaagtcctaatccacataggaaaccttcccgtacaagaaaccaactttacaaactcaaatcataccaaatttagactccttccaaatttgactccacatcctatacgcacacaatatttccattgtatgccatatgtaatcttcaccaaccacgtgcatttatttttagcctaagtatcccgcatgatatctgacggtccggacgtcaccttatctccaagttgactcccgatccatcgccgacaatactctcccgaggcatcaaaacacctacacatgaatcaaacaaagaaaccatattccaagaccaagctatctccaacttgactcattattagcaaacaacagtattacatacgcatagtatccatctagaagttataagcatgaaacatccacagatataaaaaaaacaacccgaaaccgaaaccaacacagagtcggccgatcagaccgcgggctggccggtctgaccactcacataactctggtctgaccggcaacccatgcccggtctgaccggaccaaaactctagtagcacatgttcatcacctgcaaatccaatcatctccaaaatcacttcaccaataatctcctattatcaaaaccagtaatctcagatgccaattgttcatcatagaataagaatgaaacacactttgatttacacttttcaccttggaatcaaagatttaatgcaaattaagcttcacgttcctagtttgagcttccaatttgtacatatttgtgatttataatgctatatatacatgtatacagacaagtaacgcagtccacgtgggatgttggccatgcctggataagctgagcaagatttagacctgtgtcaaaacaatgtcttgaattccgttgaactttttgtttacaggttcatcaaagatgagctacaaacgatacaagcatttttgattgctgccgaagcatcaaagaaaagcatactattgaaggtttgggtgcagcaagtaagggatctttcctatgacattgaagattgccttgatgaatttacagttcatgtgggcagccaaaacttgtcgaggcagttgatgaagctaaaggatcgccatcggattgccatccagatccgcaatctcaggacaagaattgaagaagtaagcactaggaacatacgctacaacttaatagagaatgacctcacctgcaccactactgatgagaggaatttatttatggaagacattcgcaaccaataagctaacaacattgaggaagctgatcttgtgggtttttctggacccaaaagagagttgcttgatcttatagatgtccatgccaatgacggacctacaaaagttgtatgtgttgtcggtatgggtggtttgggtaagactactattgcaaggaaaatttatgaaagcaaagaggacattgcaaagaatttttcttgctgtgcttggattactgtttcacagtcctttgttagggtggaactactcaaggatttgatggtgaaactttttggagaggaagtactgaagaagcggctgagagaactcgaagggaaggttccacaagtagacgacctcgccagctacctcaggacagagttaaatgaaaggaggtactttgttgtgcttgataacgtgtggagtacagattcatggaaatggattaatagtattgccttccctagaaataacaataaagggagccgggtgatagtaacaacaagagatgttggcttaactaaggagtgtacttctgaattgcttatctaccagcttaaacccctagaaataaactatgcaaaagagttgcttctacggaaagcaaataaagcaataggagatatggaaagtgataaaaagatgagtgacattataactaaaataataaagaagtgtggctatttaccgctggctatactcacaataggaggcgtgctttccaccaaagagataagagagtgggaaactttttatagtcagataccttcagagcttgagagcaacccaaaccttgaagcaatgagaaggatagtgaccctaagttacaactacttaccgtctcatcttaagcaatgctttttgtatctaagcatatttcctgaggattttgaaattaataggaaccgtctggtaaatagatggatggcagaggggtttattaaagctaggactaatatgactattgaagatgttgggaaaagttactttaaagaacttatcaaccgtagcatgattcagccatcaagagcgggtatacgaggagattttaagagctgtcgagtccacgacatcatgcgtgatattacaatttcgatttctagagaagaaaatttcacactcttacccgatggcactgactatgatgtagtacatgggaacactcggcacatagaatttcacgggagtaagtattgctctgaaacaagcttggactggagcattatacggtcattaactatgtttggtgagaggtccgtagaactagagcattcagtttgttcatctcagttgaggatgttacgggtcttggatctaacagatgcacaattttctatcacacaaaatgatgtcgacaacatagtgctcttgtgccacttgaaatacctatgcattaagatacagataccgttcaccatatatttattcacttccacaatccatagctagactgcatggtctgcagacattggacttgggtcagacgtacatttcaacactgccaactcagattactaacttcggagtctccgtagccttcgatgcatgaaagaatatttttcttcttctttaacaacatatttaactaacacattatgcctgcccatgatattcacacctttcgttagtacctcggatcgttctgaaacaattgctaaattgcacatggccaccaaaggcttccgttcaaaatcaaatggtgtcaaggtaccaaaaggaatatgtaagttgagagacttacaagaggattgctacggtccagcaggttgtaccgggcggtactggtaccgcgcggtaccaaaacccatctaaccgttgaatccgggatgggtaggatcgggagagaaaagatgagcaagggtggatgagggagtacctgtttcgtgtcgtcgttcccggcggcggcggtgtggagtacctgtttcgagtcgtcgttcccggcggcggcggcgtggagtacctgtttcgagtcgtcgtcattcccggtggcggcgcagagcaacaagggacgccggcggcgcgggagaggataaagtccggcggcggcgcgagagagaaaaaagggaacggcgacggtgcgggagaggaacaagggaaggacggcggcggcggaagaggaacaagtccgacggcgaggaagaggaacacggcggcggcgaaaatcatccagcgtagctagggttcgagccgcccgatccaaacccatctattgcacgcgaagttactcttttacccttccaactctcttctccatgcggtatcacctaagggacatttttggtaccgtgcggtaccacgcaacatcagccgttggatcaggccagatccaacggccagcatttggtaccgctcggtacgttggacagtaaaaaaactcgacttacaaatattggaggtagtggatattagaaggactagcagtagagcaatcaaagagttggggcagttaagcaagctgaggaaattatgtgtggtaacaaagggatccacaaaggaaaaatgtgagatactctatacagctatccagaagctctgtttcctacaatctctccatgtgaatgctgtgggattttcaggtattggaacacttcagtgtatagattctatttcatctcctcctcccctactgaggacactcaggttgaatggaagtcttgaggagatgcctaactggattgagcagctcacgcacctgatgaagttcaacttatggaggagcaaactaaaagaaggtaaaaccatgttggtacttgcggcgttgcccaacctcatggtcctttatcttcattccaatgcttaccatggggagaagctagtattcaaaatgggagcattcccaaatcttagaacattttcgatttacaatttggagcagctaagagagattagatttgaggacggcagctcaatcttgttggaaaagatagaaatattcaggggttggaatcagggattgttggtatcattcaccttccaaggctcaaggagatttcacttggatacggaagtaaagtggctaggcttggtcagctggagggagaagtgcgcacacacccaaatcaccccgtgctgcgaatgagggaggaccgaagtgatcacgaccttgcttgtgacgccgaaggatcccctgttgaagtggaagcaacagatcctgtgagagctcacagttgcaggtgatcacgttgacaacgaacgacaggtcagtcactccctacacggcatcttaatgaacttgttttatcctcttgtgagatcgatgattttaactcaccctttcatctctctcgttttcttaacctaacagcgaagagataagcacaacttaagctggtttgatcaagtgatgatctcctcctccattggcatctccggtcgtccctgcttctgcggctgcgcacctcgctgctccgaggaggggtgctgatctaaggaggcttccactttcttcaattgcgtctcatgctctcgattcttccctctcgggtatgaattgttcaatctgatattttctcgcgatctgctactggttccagcatgagcatttgaaccagcagcttagaattatcgtttgatcaggtgttatttatcccttcttacctgggaactctacttatccatttcattcagaacagaaaccatgtttattacactatagaggggaacaacagatcaggcacgagttgtggttttgttatttcctttttggtgtgcacaccaggtgattgctagaatgtctgaaagagcttgtgtgcatggttggctcaacattatgtgctaatcactcttttataccgttgctgacctatcacatagtgcaggagtaaaattttgctatgtttattgttacttaatgttgattttccttttcgtaaagaatattaactttttagataacaaaaagaatattatgtatcctgcaaaaaggtttgatttggtgatcactggctagtacaaaagtaacgagagatcagaaaaaacaaagttttggttacactagctcattacaatttatttgctgaacatgtgaaaaattaataattgtcgtacgtcatggttctctggaagtctggctgcaggtggttagttttgtcatatccattatcttggtctaccttttcctactaaactaggactatgagtagaaaaaagtaattgttggttccaagttcaaaagataggcagatgtgagctaaggaacttgcagttcttgatctccttcagagcttctcacctcacataaatggatctctgtttatcccaaagcaacattttttaacctgctagttccaaattgatcatctttgttattcttcatttattctatttctctccctaagggagttttctagggtggatatgtggaaaatcgaatgcagctatgctgttcaaaccactgtcaaaagatgcaggacaggatgggcagatgtttattcagtgttcttgttcaaatgaaacatatgctattctggaagaggttggttaacttacattgtatataacccttaccttgttactatcctcttgcaaaatgcatggattggtgaaatcatgttttttgtcatgcagaattgttttctgaagctcaaagttgaagctgcagtcttcactgtttctggcagaacccaaattggtggcttggtgctgcataacctgcagagaacgcaacggccactgcttgtagccttcaaggatgctacattattgatcatctcactacggtcccgatcaaacttctgatcgagttcgccaccgctgtggactttgcatatgttgatggagtggtgcgtatgaatccagggggcagagccactgccctgcgaccttggggcttgtcaacgagcatacagtataatattttgctgttctttcagtgattaaaaacgaaaattttaaagaaaacattgaatgcaaattagatctgcccaggttctcaaaatttctttagctccgccactgcttgaatcgatgtcatatgctgcaaattaagtggattggagatgtgcatatgctcgaatgttctcggttatattgtaattgtggggtagcaactttatcttgggtacaaccatggaacaagggtgaaattgtactaattcttatttgtgtacaattgatatatctcattacttcttgcattctgttagtcatatgtatttccatacatcgtttgcacctgctatggctgcttgaggatatggcaaagcttaaaagatgatgttaacatggtcacatggagatgcagggcactccattttctgtttttctctcaatcttatgtagagttaatatatggacagtagcaatagttacatctttctgtcaactaggcatactacccacgtgttgctccgggtctttttgggatggtctctattaagatgtaaacttatttattaaatgaattcaatcgcgtcgcattggttcatttatttagacttggttgtgatatactcctgttgtaaaatataataacttttacgattctagcaccatttataatatttataagagtacctgtctcagcaatcatcaatcattttctatttagttctttctatcttacccttacatacctttcaacactcatccattcctctggtgaataactaaaaatgtttaaatttcagagcggaggtagcaataagttctagtaaaagctgttgaatagtcccacattggttgtaaaaggacaaatgacctaacatataagtgggtgagccctgtacctcattagctagctttttgggtgaggtccctttacgatcttataattggtattagagcctggctagtttgacatttacccgagggcactactagaaaaaggaccgaccgccgctagaccaacaaaggaacatagacgagatcgccccaaaaaaagcccccacaaccaacacaaagcccaactcctaaagcgtgcttgcaccaatcgttcgagagatttcggctaggggatgccaaaatgacgtcttcgagaaaagaagcgatggaaaaccgccgccgccgtctgtcggggctcaaaggagccaagactgggctttcgcccggcaaccacccttgagggataagacatcacgacaacgccctcaggaagtcaggagggggaattaaccatcgttgtcggtccggccaaggccgggctgggttttcacctgccgctcaccacctgcgaatccacggctgacgcaccgatgctccaccaccactcaacctctgccgccatgtgggaccactgcaccggcgccccctgtcagccaaccttcatgcgccgaagaccgtgccacacccaccgacagcacctcctcgcactgagactgcctcctccactaccgcccaagcctctcgcgccaagccggccttctctactggacgcgcctctcgcgccaatccaaccttcctccatcggccgcgcctctcgcgccaagccggcctccatctcctccgcccgcgcctctcgcgccgagccagcctccgctgccagcagttgcgcctccctgcaccaagccggctttcgacccctcctccaaaggctaccgcaccgaccggatacggccgtctgccacgcccccggctagccgtccgagaccgccatgcctccccctatggcggtggcgatcgccaccaactagggttgaaagtgattcggataatttccgtccgaccggaccttttttcggattcggatagtgtcggtcggatatattcggaaatttgaatttgaaatcatgacaacttcaaatagcatttttaaatactaaatgatttcaactgaaaaagtcatcaacaacaaagttgtataactcatcaagatttataacttttattttggtcatttcttcatccgataaagtgatagtaatattattcacaaaatttacatctctcattaggttttatgaactataagagagatatataaattttatgaacaatgttactattactttgtagaacataaaagttgtagaactcatcaagaaataaaccttttattttggtcatttttctaaaagtttgaatttgaatttgaaaatatgacaacttcaaataatattttcaaatacttaatgatttcaactgaaaagtcatcaacaacaaagttgtataacttatcaagatctataacttttattttagtcattttcttcgtaagacaaattgacactaacattgttcacaaaatttacatctcttatttggttttatgaactataagagagatatataaattttgtgaacaatattactatcactttatcagatgaagaaatgaccaaaataaaagttgtattccttgatgagttctacaacttttatgttcgtgactttttcagctgaaattatttactgcttcaaaatatcatttgaagttttgaaattcaactttttaattgataaaacaaagtcacaagaaaaaatggccaaaataatagcagtaaaaacacaataacatgatagagcatgattttagaaacatttaggaaaaagaatcatccaatttggagttcatatgagtgagataaactagtttcaaatttttaaattttattttcgcatacggctccttaagacgtccgtatggaaaaattgatttttccacgcgggctcttaagttgtccgcacgcaaaatgagctcattttggcgtcttgaggagtcgtatgcgaaaatgccgacgcggcaagttgtgatccatttggaaaaatcatagggtctcgtacaaaagaaattgtttgtgtagtagcgaggggtttttatattccgattaatattcatcaccgtattcgttttgctccgtatttgtattcgataatattccatttcgtttttatatccgggtttccagttccgaaaaaaaaagaaagtgaatacgatagagctagtttccgaccatattcgatccgttttcatccctaccaccaccgcagccactactacccttccatccccgccgccatcttgccatcctcccgcaccttctcgcagtcatcgagctccgacggcacacggcgcagacggcccaccgtggtagcccgcactgccgtcgccacgaactcctcgccaccaccgcctcgaccgccagactccttcgggcgctgggtctgccgtcggcgcggccaggttcgcctcaccgacgccatcccctcgccacccccaccaccagacgctgccgaaggctgccatcccctccccttgcctcccctgccgccatcgccatccccgccactagacgccgccgccggccaccatcccgccagatccaggtgcggatctggcggtttcctccgtcgccgtaaacgcctcgaacgccgctgccaccaccaccagacaccaccgccgcaccgctcagccctgctgccagctgccccatcgccagatccggccgggcggcacagatctgggctgttctgctgccccgagcaggcccccctcctatgcccgagcacgaggatgaagccccgccgccactgtctttgtggccgcgcgactttgccggcgactgcttgggcagcggcgaggcagaggagggaaagggagatgagcaccggcgaggtcgtcgcctcccagttgcccgtggggaggggcgacacgagaggccaagcgctactcaactgcctgatgctcatccaaagtgagaaagatgcttgaagctgtcgctcgaagcaactttcagtcctcgatataattcgatataagtgatttctctctccatatttgtttggagaaatgctagttataagaaactaagtgtgaccatgtgttatagatgtcagagaaaacagttcattttctcaattctcaaggtaattgggaaaatggagaattactgagcgatacgtgttgctggaaaattgagaatcactgatgatcgcctccatctgaaaattaccgagcgatatttattgctgccaaatcaaaatgattctattcaacccgtgccatggcatggccggatggccccagttcactgattgatcacttaatatatggactgaacaataaatcatggtactttggttgtcgagcatgatcattatttgtggaccacagattcaccgaattaattgggatattgaatcggtcacacacacgacgggcgtactccgctcgtcttaaaataaatggattggacgtgacattatctactacaacgaatctggattagatagtgtctcatccaatcctaaattggtttattttgtgacggagggagtatgatgttaacgtagatggaaatgaggaattgagtagacagtgtggggctggaaaataatggaggacagtaacatcttggagtgtagtgaggcctggagggtcgtccttgacatccaaaccgcacctaactctatgataagcatcctctctcagattgttcagtgcaaaagctaccaatactgctccgagagccagaaaaaagcgctggtcgcctagtgctatcttctatgcagtcgtgagattggttgctctaagcttgaagggagagtcgaacgagtccatggcggagacggtgctgagcatggcgaggtcgctggtgggcagcgccatcagcaaggccgcctccgctgctgccgacgagaccagcctcctgctgggcgtcgagaaagacatctggtacgtactgcgtgactctcgttaatttattctgtagatgctcaggaatcagcaactattgtgttgatttccatcgtagcatatcgattttgttggccaccaattctaatcggccggaacaagctagtcactaaatctggcaaatcgatcagctgctgagtgcacaaacatgcatgttattcttttttttttttgggttatatgttaagcaacaaagccccttggtaagatatgcatggcaaatgaactaatatcgacatacgtaaagcggaggacccctcgttccatgcgtgggtgactcgagcggtgacaaatcctagcacctccacctccttggatggcctgtggtgacgctttcggccccgagtttcccttgaatacatcatctacaaggtgctattaatgtctagtcacatcatttacgaggcgctattattgcctagtctgcccgaagatagtttagacaacactcttggatggcggtgtccttcgcccagtgatgtccaagagcccgtggatgtttagttgtttagacatggtgttgggtggtgcactagtgggcctgatgggccagttgtaggtccagtggtaaccaatcatgcttagcaatagccagatgcccggattggtgcttgttcttttttcggtgtcgacgcatggtagtatttacttttcctgtttttcctgattatagcatcctaggctatactcttctaatttattcatgctatattaatattaaaacttggtatggtttgtttcattcaagacccttggtggtcaaaggcttgtttggttcaagttcattcctagccttaccaactttttggcaatagcaagaaatggtcattgaaaaaaaaaaggcaaaaattggctaggcctacagtttatttcctagcaaagttatactttagcattccactaagccaaataattcggcaatgccattttcttatctacatgccaaatatatggctaatattttggcattaattactcttattttttttggcaaaattgatcaaaagttcacatttttagctctatagtatcaaaagttatctattcactttaatagaccgaaagtttactcggttcccgttttcagcactaccgtctcttttctcttgatttgccgtcaattttgaccggcagtcctacccccaggagacattgagcagcagcccgtgatccccctctctcgccgccggtgacgctgtggtggcatacctgctgcgggcagaataagtctggcgtcatcgccctatcgcctggagctgcaaccaccactaccgggcccatcgatcgtctagagcgttatccaccctgcctgccccattacttgcagctccggctgggtcagaacctctccatgaattttcagaatatgccatcgaatacgcgctgcttttaagatatgctacctgattcgtgctattttcagaatacgctattggaacacgaattttcttcgttccgtgccactccgtctctcagagtcagtcgtgccgtcgtcatccgtctgcctagcactgtcgtcgtcagtccgccacccgtgcctgactgcccgttcagctgcgccgtcgtccgtccgtcgccgccattgccgtcgtccactgtcgcgcccgcacctgcacccgtgtcaggcacgccgtcgtccgtccgctgtcaccatcatcgtcgtccactgcggcgagcgcagacagctgtggacggatgattgcgcggcaagcgcaagcggcggtggacggacgacggggcggtgcacgcgagcacggtagccgatggactgacgttggcggcattgggagatggacgacgacggcatgattgacgtggggaacggaatgtcacggaacggagaaaattcgcactccggtggcgtatattccgaaaatagcatgaattgggtggcatatcctaaaaacagtgtctattcggtggcatattctaaaaattctcgatcagcataatccccatcaatccccaatccctcaacagttgggttaatattcctggagatgtgttcggttgtttaggttgaagttctccacttcacctccatgagtacatgcacctctacacgtacgttcttaatgagtttgtttgttctatcctccgcttgggttctattttgttggttccgatctgatttgatctggagcggggtcgatcttccacgacggcgagagagacgttgttcgggctgctcgatttggttcaactgtttaggtcgaagggaggggtagaattgcaattcaagtgcatggtcagtcaatttgggtcaaaattaacatcaaactgggataaagagacgacagtgccaaaattggtaaccggaaaactttgagttctattaaagtgaaccagtaactttcgttgctatagaataaaaacgtaaacttttgatgctccttggttgagcttggtacaaaccaaacagacgtaaaataaacactatcctgaatcaagtctactaagttccattgaactcaaccaggatacgtacacttcctcttagaagatgtcttgttttcactttgtaccattttttctattgtaaatttggtacctcgttgtacctaggtacaagaggtaccatgaggtaccattttttctattgtaaatttagtacctcgtggtacctaggtacaatgaggtaccaaattttacactaaaattttggtaccttatgatacctcctcaaggaccgtagaattgctcttaatttaattttaaaaaaaacataatatttttaaagcatattatggaaattttagtaattattacttttgtaatatatgagttacggttatactcgagataccctaaattgcttggagatgaataattacaaggtatatcaaagatgagttgaaaataatgcaggcattccttagagctgcagaagttatgaaaaagaaagacgaactattaaaggtttgggcagagcaaatacgtgacctgttatatgacattgaagattcccttgatgaatttaaggtccatgttgaaagtcaaaccctatttcgtcagttggtgaaacttagatagcgtcaccggatcgctagcccgtggatgtttagttgtttgcacatggtgctggatggtgcgctcatggtcttgttgtaggtctggtaccaaccagtcatgcttagaaatagccggatcagtgcacggtgctaggactttacttggtggtctgtgcagcgctatcgacatgtggtggtgtgctttttttttttccggattacaatctcatagggctacactctagttattttgctgctatattaatatgaaaacttggtatggttcgtttcttttagaaaaaaacctagttgatcaagggctagttttcttcaagtgcattcctaatcttagcttctttttttttttgcaatggcaagaattgttcattaaaaaaattgataaaaattggctaggcctacgttttgtttcttaccaaagttgtactttaacaataaactaaggcaaatatttcggcaatgccattttcttgtctacagaccaaatatatggctaaattttggcataaccatttttttgtttgcttggttgagcttggtacaaaccaaacagacccaaaataaacagtgtcatgaatcacgtctactaaattcctttgaactgaactagaatatagttgctcttaaaagatttcttgatttcactcggtaccatttactagtacaaacttaagatttaatttttaaaaataaaatcataatattgttattatggaaaatttagtcatagtacttttgtaatatatgagatgggttatacttgagatatcctaaattgctttaagatgaataattgctaggtatatcaaagatgagctaaaaacgatgcaagcattccttagagctgctgaacttatgaaaaagaaagatgaactattaaaggtttgggcagagcaaatacgtgacctgtcatatgacattgaagattcccttgatgaatttaaggtccatattgaaagccaaaccctatttcgtcagttggtgaaactcagagaacgccaccgaattgctatccgtatccacaaccttaaatcaagagttgaagaagtgagtagcaggaacacacgctacagtttagtcaagcctatttcctctggcacagagattgacatggattcctatgcagaagacattcgtaatcagtcagctcgcaatgtggatgaagctgagcttgttgggttttctgactccaagaaaaggttgcttgaaatgatcgataccaatgctaatgatggtccggccaaagtaatctgtgttgttgggatgggtggtttaggcaagacagctctttcgaggaagatctttgaaagcgaagaagacattaggaagaacttcccttgcaatgcttggattacagtgtcacaatcatttcacaggattgagctactcaaagatatgatacgccaacttctaggtcccaattctctgaaacaactcttgcaagaattgcaagggaaggtggtggtgcaagtacatcatctttctgagtacctgatagaagagctcaaggagaagaggtactttgttattctagatgatctatggattttacatgattggaattggataaatgaaattgcatttcctaagaacaataagaagggcagtcgaatagtaataaccactcggaatgttgatctagcggagaagtgtgccacagcctcactggtgtaccaccttgatttcttgcagatgaacgatgccataacattgctactaagaaaaacaaataaaaatcatgaagacatggaatcaaataaaaatatgcaaaagatggttgaacgaattgtaaataaatgtggtcgtctaccattagcaatacttacaataggagctgtgcttgcaactaaacatgtgtcagaatgggagaaattctatgaacagcttccttcagaactagaaataaacccaagcctggaagctttgaggagaatggtgaccctaggttacaaccacctaccatcccatctgaaaccatgctttttgtatctaagtatctttcctgaggattttgaaatcaaaaggaatcgtctggtaggtagatggatagcagaagggtttgttagaccgcaggttgggatgacgactaaggatgtcggagaaagttactttaatgagctaatcagccgaagtatgattcaacgatcaagagtgggcatatcaggaaaaattaagacttgtcgaatccatgatatcatccgtgatatcacagtttcaatctcgagacaggaaaattttgtattgttaccaatgggagatggctctgatttagttcaggaaaacactcgccacatagcattccatgggagtatgtcctgcaaaacaggattggattggagcattattcgatcattagctatttttggtgacagacccaagagtctagcacatgcagtttgtctagatcaattgaggatgttacgggtcttggatcttgaagatgtgacattcttaatcactcaaaaagatttcgaccgtattgcattgttgtgccacttgaaatacttgagtattggatattcgtcatccatatattcacttcccagatccattggtaaactacagggcctacaaactttgaacatgctgagaacatacattgcagcactaccaagtgagatcagtaaactccaatgtctgcatactcttcgttgtagtagaaagtttgtttatgacaactttagtctaaaccacccaatgaagtgcataactaacacaatatgcctgcctaaagtattcacacctttagttagtcgcgatgatcgtgcaaaacaaattgctgaattgcacatggccaccaaaagttgctggtctgaatcattcggtgtgaaggtacccaaaggaataggtaagttgcgagacttgcaggttctagagtatgtagatatcaggcggaccagtagtagagcaatcaaagagctggggcacttaagcaagttgaggaaattaggtgtgataacaaaaggctcgacaaaggaaaaatgtaagatactttatgcagccattgagaagctctcttccctccaatctctctatgtgaatgctgcgttattatcagatattgaaacacttgagtgcctagattctatttcatctcctcctcccctactgaggacactcgggttgaatggaagtcttgaagagatgcctaactggattgagcagctcactcacctgaagaagatctacttattgaggagcaaactaaaggaaggtaaaaccatgctgatacttggggcattgcccaacctcatggtcctttatctttattggaatgcttaccttggggagaagctagtattcaaaacgggagcattcccaaatcttagaacacttcgtatttacgaattggatcagctaagagagatgagatttgaggatggcagctcacccctgttggaaaagatagaaatctcttgctgcaggttggaatcagggattattggtatcattcaccttccaaggctcaaggagatttcacttgaatacaaaagtaaagtggctaggcttggtcagctggagggagaagtgaacacacacccaaatcgccccgtgctgcgaatggacagtgaccgaagggatcacgacctgggggctgaagccgaaggatcttctatagaagtgcaaacagcagatcctgttcctgatgccgaaggatcagtcactgtagcagtggaagcaacggatccccttcccgagcaggagggagagagctcgcagtcgcaggtgatcacgttgacgacgaacgataggtcagtcactccctacatggcagcttaattaacttgtttctaattctcttcttgttcagtattagccatcaggtgagggcgatgatttcaactcacttttcatctctctcgttttcttaacctgacagcgaagagataggcacagctcaagctggctgacgatctcctcccccatcagcgtcgtcatcagcgaacagatagggcagggcttccctgcttctgcgtgcacctcactgctctgactctgagggacatgatgatcaatgaggcttccagttcccaaatgtgcgcctaacacactcagtcattcccatccaaggtatgaattgatcgatctgatttatttcttgccgtgaggtgcaaatgtttgatgcttactttgatcagggtattctgtttcgggctgtgatatgcacaattactctcaattcgtgttttgatgctgagtttttatttctcttcttacccgtgcactcttcatttccatttcattcataacagaaaacaatttgattgcattgtggaagggaatatgagatcagaaatcagatggttagttgtggttttcttatttcgtttgctatgagcaccaatcgtttgctagaatgtctgaaagatcttgtgtacatatggtggactgaacaattgaacattacaagttatcatattttatattgttgctaaccgatcacatagtccagtagtccagtagtaagatttttgttaagtttattgttactgaatatattgtttggcctgcagttgttatttctctcaaaacaaaaataatttgttagtctcaaatacaaaaagaatgacagatcaaacaagttgtttcactctactagtttcaaattgatcatctctgtttgttcttcattcattttctctctgtaagagagtttgctagggtggagatgcggtcatgtggatgatcaaatggactacatcagacgcatcacactgctgcccaacctttacgcactgttgacaaatggagtgcaggtcctaaaccaggccagaagtttgttcagtgttcatgttcccaaacatacattctggatggcaggttaatttattataacattcactcttatagctttcttagtcaaaattacaaataaggtctcctaaaaaatgcatcgacgttgatatctgtgttttctgccatgcagaatgacttgctctcaatggttgaagctgcaatcttccaggctcatttctggctgaacccaaattggtggcttggaggctggagctgcatgacatcagagatcacaatggccactttttgttgccatgggtgagggtacatggatcatgccgctgagcctttactccgagctagcagaaaactggctggctgtagatcgagttcgccatcgccctcaactttgtcgatgtgatggtgatcatggatagatgtatgtcacagcgcaaagcggagactccaatgttcgcaaccatactgtaaatgtgggaggggaaaatttctcaaggggacaaccatggaacagaggagatggagcaaagtagttgctcttgctatcatttcagagctcaggctgatctctagctcagataggagtactaattcttatgtgtgtgtatttgatctatgatggtacgttttaagagttggaccagatattgcttgatctttacatgatgaattgatgatatgcaaaaaatggtatttgacatggagacgcaggaacagggtggttcattgatctgtttgatcttaatcttggagagctaggatggaaaactgagttggtggttatatctctactacttttgatgttgtccaaaactaatttgtttctactcatgtacgtatgtaaaaaggaaaattttaatgattaaagaacttattgttctctttaatcagattatttatcagtgggatttttctttctctggtctgtataaaaatactttttttatcgtcaatcctcctaaaattcctatttataaactattttttcttcagtttttttcctgtattttggttttctttcctctacgtgactacacattttgaatcgaacatgctctgtattcggccaacttagggccatttgaaatgtgcactggacagttgtttagaacacttccaaggaatttctcgtcagttgtcggccgacggctatttaaacctgatttaagaattccatgcgcagagacttgtactagcgtcgaagacttgcttcggagactcggagtcaagtcaaggccaaggccaccacagcgtcttcgacgacgaagactcgggttaatgcttcttcgacgacgacgacgacgccagccacggcgagccaagccaaccaccgcggacctgagggccccggccaagcgcacccacgaaccaagtaccaaccacgccattgcttttgtctacaaaagacatccgcacattccttcctttccccattgttgctcagccactcggcttcggcttttcctccagctccaccaacctttcccgagattagagagggaagaggagcacgccgaaggatcacggtcgaacacctactatgcgcacggccgtgtgtgcacagatccattatcccactagtgtgtgcaaagcaaagcagcataggtaagtaactccgattttctgagctttgctatttaattaagcagggaagattgtttgggtattttccgcagttttctggaaaccatttttttccccaatcatttccatgtttagctatttttcacgcctgtctgttaatctaatcctgcaaaacatagcataaagttgtaatacaagtacatcgccaatctacaacttttttttaaaagttagaacattcaccccaaggaagaaaaggtaaacttatgaattgttggacttggactaatcttatatttttcccaaagacttggaggaatgttaaggaatatcactcggactgtccctcctttttatttgcatttcctcaagattttttttttggggcatgtttgttgtttggctaatgggttagagaaatgatttcccacttaccaaaagacatctttaaatcctaaccattcattgtccctttttcaattaatcctaacccttcattcattttccaatcccaatttcacctctcattttccattatccaaacatagccacggagcttgtttgtgtatattctgttaaaccagcggttttgagacactaattaagcaaatttactagttaagagattataacgtttgagtttgggctttggagatgtcactggtggtcgaaggtacgcaagccctttctgctttgctacgaagaacctccgagctcagtcaatgtcaggaaaacaaactttgataactacaagtggaagccatttttggttggttttatgcatgatctgtttgtccctgttattatgtcctgcaaacttttcattgcccggtttgattaatacatgtctatttgatattgcagataaatacagttgtagcctgaaagatagaacttacaatctattgcttaaaagtttaaagaaagttgttcagagaaggaagatcagatggcggatacagtactcagcattgcaaagtccctggtgggaagtgctgtaagtaaggttgcttcggttgctgccgacaagatgatcttgctgctgggagtgcagaaggagatatggtgagcatctgaatcgcagcctaattaattttattttccagttgcattagattttttgggaccacacttatgcagagtggtatggtactcagtttatttttagtgatttatttatatttttataccatatttgatgaatgcatatgcagtttttttttaataagtatattatttgcaacatcttcgggagatacatgtagaggaaagtcaaagtagaatgaaatggaagttcctctatataccctgtaggatcgaaatagaagaactaagccaaccagaggggagggggtgaatggttagtatacccaaaaacgtaaactttttgcagaaattaaagttaccctcagattcgatgtagatcggtctgaccggagtagtctcgtcaggtctgaccgcgcctgtgccgccggtctgaccgcagtaggttgcccggtccgaccgctctgtagtcgccttagcctcctgatgctgatgctgctgtgaccgtgtcacgcccggaatttctatccaaaattccaaacgcttacatgtgtgtgaacccttgtccaggaatcagccgaggcacacaataacaaattgataatagagtacaattattactctaattaataagcgtataaaatgtcattacagaggtagatagttcctctcaaacaataaagatctaaacagcggaaaaagataaacggcgcagacggctccactccacaggcagcttgaccaaggctacacctaatcctccacaccatcagcttcactgtagaactcttcctctgatgaatgattgcaaggtgagtatatgacatactcagcaagccacgcagcaaatatgcaagtgcacaggataacaaaggatggcatagtagggtttcttttgcaaaagcagcatttagcaaacatttgagaatttaataaaacagttaagtaattaaacaatattaatccaacgctatacaacataccctgttgcataggcccaaccattctgaacaaccaccccggctgcacagttctatctccaaaccaggaatataccattccaaaccaggagctaatcaaatcattaccaattatagcatcattaactatggtgagaagggtgagactaatcacgaaagacattgttagacccgcccataaccgcgggcacggctattcgaatagttttactctgatcagaggtgtaccactgtacccacaagacacaaccccacatcatgtcaccatgtgcctcaataccaccacggtacctcggaaaggagttgtgacaatacccctcgcataacacaatccactgcagtgcatcttcctggatcataatcacccccttataaacaaggcatggactccccagcgacccccgtgggcttatctccgccacttctcagtctggtgctccacaatgaaccatactatacaaaagataaagccgttgcccatgctggcttgtggttggcacgataaatgtttcacaaccgaaactcgtgaaccggtccttaattgtcatgagcacgaccatcaaaaccatgtgctcacaacccaccattatcaggttttagttggcacattaattaattaactaatcacaattgaccatcgtgaactatcaataagccatcatgaaataatagtgaatcataagttatcccaatagtgagctaatgtttctaagcagggctaagcaattatatctaatatctagttgaaccaatacataaagctcaactagtcaaattattataacccaaggtatcaaggaataaggtaaacaagaacaaaagggctataacaaagaataggttaattccacccaatgacattcgaaaaataaatgcaatagttgaataaaaacaatagctttaaataggatcaacatgctcaaagggttgtttgggatctgtgtgacttgccttgctggccttggaactcttcaaactcttctccggcgaaaacggactctccggaaacgacggaatctaagcagaaaagagcaaaatcaccaaaacagcacataaacaagcatgaacagtacatgtggatatttttaatgtgtagatctcaattttagaaaaatttagagacttgaaccaactaaatcggagctaagatgaattagttatgaatttttaaagattaaatcggattaaaacacttaaatcggcttaaattgaattatgacgcaataacgaattatttttgaaaaggaaaaggggatttattgcgtcagcggctagggtttcggtgggccgggcgcacgggcggcggttcacgcgaacggacggccgagatctaaccgatccaagatggacggccgagatcgatcgagtgcacggcggctcacggcggaagggcatgatgacgtcagcgatgacgtcaccgacgacggcggcggctcggcggctggaacggctcgggcggcgcatgctcgccggcgaacggcggcgcgaggacgcaaacggagggcaccaacggatagcggacggcgcggcgaactcaccggcgaccaaagcgacggcggagaggcaacggacggcgacggcgacgaggaggaaacggcgaagctcttcgggttgacgacggcgacgatgctccggtggtcttcggcggcggcgaaggggcggacgaggacggcgacgcgacggcgaacacgacggcggccttcccgagcgacggcgacggctggaacgacggcggcacacggctggagcggcggcggcgacggcaaagctagggcacacggcgctagggctcttccggcgacgagaggtgaaggcgaaggtggcggcgagaagagaagagcacggggatccttttaaaggggctcggaggcgacggcggaggcccacggcgaccggcgacgaggaaggaaagctcgggaagaacgaatccgagacgaactcgaatccaacgctttccaaagcgatttaaccgatgattccaaaggagaaaaggtagaggagatcccggagattgtttcccctcaaccaattcggccggagaaggaaagggacggccggattggaaggaaacagcggcggcggctcggcggcggcgcgctagggtttcggcccgaggaagacgacgggcctgacaggtggaccccacctgtcagcggcagcgggcgcgcgcgcgcggcagcggactgggccggactgggccgaggagagagagagagggttttgggccgactttcggcccaaagccaaaagaagactttttaaaactttttcaatttaaattatttcttaaatgcaattccatttattaaaaatactttcttagctcaaataaatcccagaaaaatctaggaattatagaattaagtaaagtatttaatgaaattttatctggccccattttatattgaaatttatttattaaaattagattttcttttcaagacttttaaaaattatttctaaaaattccaattaaacaacaatttatataatttgaatttttagggtgtgacagaccgccgttgctccgccggtttgaccgcctccttgccgtcggtttgaccgccggtgagttgccggttagaccgccgaacccgataaaacacaaatcgaagaactcttaaagtagatgacaaccttattacttatctctgtgtttacaaagtgcaacaacaatactccttacaaaaaaactcgactaaactcgaaaccctaactaaactatcaactcaattgctctcaaaagcgataccgcgaagcctcacgctccctctctatttatacatgaggtaggcagcctaaagccacgaactaaactcatactagaagtcctaatccacctaagaaaccttcttgtacaagaaacaaactttacaaactccaatcataccaaatttggactcctttcaaattcgactccgcattccacacacacaatacctccatcgtatgccatatggaatcttcaccaaccacgtgcattgaactctagcctaaatatcccgcatgatatctgaccgtcacggacatcgtcttatctccaagccgactcaagatccatcaccggcgatactctcccgaggcgtcaagtcacctacacatgaatcaaacaaagaaaccgtattccgagaccaagctatctccaacttgactcattagtagcaaacaacagtataacatacgcatagtatccatctagaagccataaatatgaaacaatcacggatatccaaacaaacaacccaaaaccgaaaccgacacagagtcggccggtcagaccgcgggctggccgatctgaccgagtgatacacgtcggtctgaccagaaccgacaaatcagcagcacctgtttaacacctgcaaatccaatcatctccaaaaccacttcgcaaataaattccaaatatcaaaaccaataatctcagatgccaattgttcatcacagaataataataaaaaacacctttgattttacatacccaaacagaagtggacatagctttctcatggcaaccatgttcaatgaaggaatacaaacgagccagctatttgtaggatcgaatcacaagaactaagcctaccagaggggggtaaatggttggtatacccaaaaaccgaaaacttttagcggaaataaaagttaccctcaatttcgatgagatcggtctgaccggagtagattagccggtctgaccgaagcgtagccgccggtctgaccggtgttgatcttccggttggactgccctggaatccctgccgcgcctgatgtcgccaccggtctgaccgcaggtcacctgccggtctgaccgccgtgatgccgccggtctgaccgccggtgcgccgccggttagaccgccgaaacccggtgaaacacaaatcgaagaactcttaaagtagatgacgactttattgattctctctgtgtttacaaagtgcaccaacagcactccttacaaaaatttcgactaaactcgaaaccctaactcaaaactcaactcaattgctctcaaaagcgataccgggaagcctcacgctccccctctatttatacccaaggtaggcagtctaaagccatgaaccaaactcatactaagagtcctaaacaccttaggaaaccctctagtacaagaaagaaactttacataaccaatcgtatcaaattcggactccttccaaattcgactccgcatcccatacacatacaatacctccatcgtatgccatatagaaactccatcaaccacgtgcatcaattctagcctaagtatcctgcatgatctttgaccaccacggacgtcgtcttatccccaagccgactcccggtccatcaccgcaaatactctcccgagacatcgagtcacctacacatgaaataaacaaagaaaccatattccgagaccaagctatctccaacttgactcattagtagcaaacaatagtattacatacgtatagtatccatctagaagtcataatcatgaaataatcacggatatccaaacaaacaacccgaaaccgaaaacgacacagcgtcggctggtcagaccgcgggctggccggtctgaccgctcgatcaccgccggtctgaccggcacatactgcctggtctgaccggtcacataaaatgatagaatcctgcgatcacctgtaaaatccaatcatctccaaaaacacttcgtgaataaattccaaataacaaaaccactaatctccaatgcccaattgttcgtcacagaataataatcaaaaacaccttttattttacactattatggatctggtggtaatctaatttagacagaaacatggttgccttcgagagtaaacaatgtagcatgccatgcttaagtcaaatgcatatacaattggacaaggaactattacgtttcagtgctgttcaattttgcttttgaccttggaaccaaatatataatgcaaattaatacgttcctagtttaagcttccaatttgttcatattttgtgatttataatgctatatatgcatgtatacagacaagcaacgcagtccacgtgggatgttggccacgactggatgagctgagcaaaatttagacctgtgtcaagaaaatgtctgagttcctttgaacattttgtttacaggttcatcaaagacgagctacaaacgatacaagcatttttgatggctgccgaagcatcaaagaaaagcatactgttgaaggtttgggtgcagcaagtaagggatctatcctatgacattgaagattgccttgatgaatttacagttcatgtgggcagccaaaccttgtcgaggcagttgatgaagctaaaggatcgccatcggatcgccgttcagatccgcaatctcaggacaagaatcgaagaagtgagcagtaggaacacacgctacaacttaatagagaatgaccttaccagcaccactggtgagaggaatttcattatggaagacattcgaaatgaatcagctaacaatatcgaggaagctgagcttgtgggtttttcgggacccaaaagagagttgcttgatcttatagatgtccatgccaaggatggacctacaaaggttgtatgtgttgtcggtatgggtggtttgggtaagactactattgcaaggaaaatttatgaaagcaaagaggacattgcaaagaatttttcttgctgtgcttggattactgtttcacagtcctttgttaggttggaactactcaaggatttgatggtgaaactttttggagaggaagtactcaagaagcggctgagagaacttgaagggaaggttccacaagtagacgacctcgccagctacctcaggacagagttaaatgaaaggaggtactttgttgtgcttgatgatgtgtggagtacagattcatggaaatggattaatagtattgccttccctagaaataacaataaagggagccgggtgatagtaacaacaagagatgttggcttagctaaggagtgtacttctgaattgcttatctaccggcttaaacccctagaaataacctatgcgaaagagttgcttctaaggaaagcaaataaaaaaattgaagatatgaaaagtgataaaaagatgagtgacatcattactaaaatagtaaaaaagtgtggctatttaccactggctatactcacaataggaggcgtgcttgccaccaaagatgtaagagagtgggaaaccttttatagtcagataccttcagagcttgagagcaacccaaaccttgaagcaatgagaaggatagtgaccctaagttacaactacttgccatctcatcttaagcaatgctttttgtatctaagcatatttcctgaggattttgaaattaataggaaccgtctggtaaatagatggatggcagaggggtttattaaagctagggctaatatgactattgaagatgttgggaaatgttactttaaagaacttatcaaccgtagcatgatttagccatcaagagcgggtgtacgaggagaatttaagagctgtcgaatccacgacatcatgcgtgatattgcaatttcgatttctagagaggaaaatttcatactcttacctgagggcactgactatgacgtagtgcatgggaacactcggcacatagcatttcacgggagtaagtattgctctgaaacaagctt

SEQ ID NO: 84 - DNA, 2940 bp, Oryza minuta
  CDS (1)..(2940)
atggcggagacggtggtgagcatggcgatgtcggtgctgggcagcgccgtcgggaaggccgcctccgccgccgccgacgaggccaccctcctgctcggcgtccagaaggagatctggtacatcaaggacgagctgaaaactattcaggcattcttaagagctgctgaagtaacaaagaagaaagatgacttgctaaaggtatgggcagagcaagtacgagatctgtcatataacattgaagattgcctagacgaattcaaggttcatgttgagagccaaagcttggcaaagcaactaatgaagcttggtgaacgccatcgaattgctgtacagattctcaacttaaaatcaagaattgaagaagtgagcaacaggaatacacgctacagcttaatcaagctcatttcctctataaccacagaggatgagagggattcctacctagaagatgctcgcaatcgatcaggtagcaacactgacgagtcagaacttgtgggctttgccaagactaaagatgagttgcttaaactgatagatgtcaatactaatgacggtccagctaaagtgatatgtgtggttggaatgggtggattaggcaagactacccttgcaaggaaggcatatgaaaacaaggaacacatgaagaacttctcgtgttgtgcttggatcactgtgtctcagtcatttgacaggaaagaaattctgaaacaaatgatcaggcaacttctgggtgctgattcattagacaaactcttgaaagaatttagtgagaagttgctcgtgcaagtccagcatctcgctgatcacttggttgaagggctaaaggagaaaaggtactttgttgtccttgatgacctatggaccatagatgcatggaattggattcatgatactgcttttccgaagattaacaacagaggtagtcgcataataataacaacgcgagatgctggcttagctggaaggtgtacctctgaatcacttatttaccaccttgaaccgttacatatagatgatgctatacacttgctactagcaaagacaaacataagacttgaagacatggaaaatgatgaggacttgggcagcatagttacaaaattggtaaaaaggtgtggttatttaccgctggctatactcacaataggaggcattcttgctactaagaagataatggaatggggaaaattttacagagaacttccttcagagcttgagagcaatccaagcctagaagccatgaggaggatggtgaccctaagctacaatcacttaccatctcatcttaaaccatgctttctttacctaagtattttccctgaagattttgaaattcaaagagggcgcctggtagatagatggatagcagagggttttgtcagagccacagatggggtgaacattgaggatgttggaaatagtcactttaatgagcttatcaacagaagtctgattcagccctcaaaagttagtacagatggagttgttaagagatgtcgaatccatgatatcatgcgtgatatcatagtttcaatttctagagaggaaaattttgtgctgttgactagggagaagatcactgttgtagcggaggagagcatccgccatctagcatttcatgggagcaaatgctcaaagatatgcttggagtggaaccatctgcgctcagtaactttgtttggtgacagacctgcggggcgaacacctgcactttgttcaccacaatttaggatgctgagagtgttggatctggaagatgcaaaattcaaattcacacaaaatgatatcaggaatatagggttgttgcgccacatgaaatatttgaattttgcaagagcctcaactatttatacacttccaaggtccataggaaaattgcagtgcttgcaaattttgaacatgagggaggcaaatatctcagcactaacaactgaggtgactaaactccagaatctccgtagcctccgatgcagcaggaggtcaggttctggttactttagcataatagataatcccaaggaatgcttgatgatcaccatgtgcttaccgatggttttctcaacttcaataaatttcagtgaccgtgtgaagttaattcctgagatatgcatgtcatgttctacccgttggtctgatacaaagggtgtgagggtgccaagaggaattgacaacctaaaagagttacagattctagaagtcgtggacatcaacagaactagtaggaaggcgattgaagagctgggggagctaattcagttaagaaaattaagcgtgacaacaaaaggcgccacaaataagaagtatcagatattttgtgcagcgattgagaagctctcttctctgcaatctctccgtgtggatgctgagggattctcagatactggaacacttgagtggctcaattcgattgcatgtcctcctccattcttgaagacactcaagttgaatggatctcttgcagatacaccaaactggtttgggaaccttaagcagctggtgaagatgtgcttatccagatgtgtgctacaagatggtaaaactatggagatacttggggcactgcccaaccttatggttcttcgtctttatcgcaacgcatatgctgacgagaaaatggcattcagaaggggaacattcccaaatctcaggtgtcttgatatttacttgctgaagcaacttagagagataagatttgaggagggcacctcgccaacaatggaaagtatagaaatttatggttgcaggttggaatcagggattattggtatcaagcaccttccaagacttaagattatttcgcttgaatatgatggtaaagtcgcgaagcttgatgtgctgcaagaggaagtgaatacacaccccaatcatactgaactgcaaatggcagaggatcgaagtcatcatgacctaggaggtatattatataaggatcaaatatag

SEQ ID NO: 85 - protein, 979 aa, Oryza minuta
MAETVVSMAMSVLGSAVGKAASAAADEATLLLGVQKEIWYIKDELKTIQAFLRAAEVTKKKDDLLKVWAEQVRDLSYNIEDCLDEFKVHVESQSLAKQLMKLGERHRIAVQILNLKSRIEEVSNRNTRYSLIKLISSITTEDERDSYLEDARNRSGSNTDESELVGFAKTKDELLKLIDVNTNDGPAKVICVVGMGGLGKTTLARKAYENKEHMKNFSCCAWITVSQSFDRKEILKQMIRQLLGADSLDKLLKEFSEKLLVQVQHLADHLVEGLKEKRYFVVLDDLWTIDAWNWIHDTAFPKINNRGSRIIITTRDAGLAGRCTSESLIYHLEPLHIDDAIHLLLAKTNIRLEDMENDEDLGSIVTKLVKRCGYLPLAILTIGGILATKKIMEWGKFYRELPSELESNPSLEAMRRMVTLSYNHLPSHLKPCFLYLSIFPEDFEIQRGRLVDRWIAEGFVRATDGVNIEDVGNSHFNELINRSLIQPSKVSTDGVVKRCRIHDIMRDIIVSISREENFVLLTREKITVVAEESIRHLAFHGSKCSKICLEWNHLRSVTLFGDRPAGRTPALCSPQFRMLRVLDLEDAKFKFTQNDIRNIGLLRHMKYLNFARASTIYTLPRSIGKLQCLQILNMREANISALTTEVTKLQNLRSLRCSRRSGSGYFSIIDNPKECLMITMCLPMVFSTSINFSDRVKLIPEICMSCSTRWSDTKGVRVPRGIDNLKELQILEVVDINRTSRKAIEELGELIQLRKLSVTTKGATNKKYQIFCAAIEKLSSLQSLRVDAEGFSDTGTLEWLNSIACPPPFLKTLKLNGSLADTPNWFGNLKQLVKMCLSRCVLQDGKTMEILGALPNLMVLRLYRNAYADEKMAFRRGTFPNLRCLDIYLLKQLREIRFEEGTSPTMESIEIYGCRLESGIIGIKHLPRLKIISLEYDGKVAKLDVLQEEVNTHPNHTELQMAEDRSHHDLGGILYKDQI

SEQ ID NO: 86 - DNA, 2982 bp, Oryza minuta
  CDS (1)..(2982)
atgactgagacggtggtaagcatggccatgtcgctggtcgggagtgccatcagagtggcttcttctgcttccagccaagagatgagcatgctgattggtgtgcaaaacgagatttggttcataaaagatgagctgaagacaatgcaggcattcctgagggcggctgaagtaaggaaagatcaagatgaactcgtgaaggtgtgggctgagcaagtgagagatctagcctacgacattgaagattgtcttcaggagtttgctgtccatgttgggcatcaaagcctatcaatgcagctgattaagctctgccaccgacaccgaatcgctgtccagattcggaacctgaaacagagggttgaagaagtgagcaatagaaacatgcgttacaatctaatcaagtctgttccatctagcagtaccgatgactcccaaagcaacatggaattgattcggtatcagactgctcattatgttaatgaatcagagcttgttgggtttgacgttcccaaaaaggagatcctggagttggtatcaagcagtgaaaatgctgaagcccaaacaatttggattgtcggagaaggtggtcttgggaagactactcttgcaaagaaggtttatgaaagctcagatgttagtagcaagttcccgtgtcgtgcttggatcactgtgtcacgatcatttaatgtcaaggatttactgaagaacatgatcaagcaattactaggggaagactcactcaagaaactcttggatgaacacaaagaggtgattgtagaaaagcacaaccttacagaccacctgaacaaagggcttagagggagaaggtattttcttgttcttgatgacttgtggaccacacaagcatgggattgcattaaacctacttcctggggaaataacatggaaggtagccgagttgtagtaacaacaagaaacaaaaacctagcaggtggtagttccacctcgccagtctactgccttaaaactctagagaaagaagatgctaccaaattgttgttaagaaagacaaaaagaagtctgcatgacatagaaaaagatcaaatgaaagaaatatttgaaaagatactaaagaaatgtggaggtctaccgctagctataatcacaataggtgcagtccttgaagggaaagatataaaagagtgggaaattttgtatgctcaacttccatcagaacttgaaagcaatccaatcgctgaaccaatgaagaaggtggtaacccttagttacaattacttgccatctcatcttaagccttgctttctgtacctttgcatctttcctgaggattttgatatccaaaggaagcgcctagtacatagatggattgcagaggggtttattagagctaggggtggagtgggaattgtggatgtggcacaaaagtattttgatgagttgatcaaccgaagtatgattcaagcatctagagtggatatagagggaaatattaagagctgccgagtccatgatatcatgcgtgatgtcatgatatcaatatctagggaagaaaattttgtatacttgatgggtgatgatggaactagtgtagtggaggaaaatattcgccatttagtgcaccatgatactagcaagtgttcaaatataggcatggattggagccatgtacggtcattaactttgtttggcaatgagagacccaaagggctatctccttcattttgttttccccaattgaagatgctaagagtgctggatcttcaagatgtcaaatttgggatgacacaaaaagatatcggaaaaatagggttgctgcgtcacttgaaatatgtgaatattggagggcattcaagtatttatgcacttcctaggtgtataggaaaattaaaagacttgtgcactttggacataactgacagttacattacagaactaccaactgagattagtaaattgcagagtctatgtattctccgttgtagaggaagaccaaactcgggggattttaatctaaatgatcctaaggattgtctaattgccttctcatgtttgcctctgcttatggctgcaaccgattctgatgaacgtaataaaataattgctgagctacacgtgggttgttcaagtcaatggtctcctaatggtggtacatatggtgtgagggtacctagaggaatcaagaatttgaaaaggctgcaggtgctagagacagtggatatcaatcgaaccagcagtaagtcagttgaagagttgggagagcttatccagctaagaaaactaagtgtagtaacacaagggtccaccaaggaaaaatgcaaaatactctgtacagccattcagaagctaacttccctcaaaactctctatctgaatgctcatggacccttggatactggaacacttgaatggctacattctatttcccatcttccttccctgaggatcatcagattgatcggatacatgaaagagatgcccaactggtttagggagctcagacagctggtgaagattcacttgcagaatagccaactagaggaggataaaacaatggagatactcggagaactgcctaatctcatgctccttttccttagttggcgagcggtgctaatcattcgcaatcagaagcaactgaaagaggtgagatttgaagagggaacctcaccctggatggaaaggattgatatcagagaatgcagattgacatcagggattgctggtatcaaacaccttccgaggcttaaggagatttcacttgaatacagtgctaaagtggtgaggctaggtcagctagaggttgaaatgggcacacaccccaatcgacccatgttgcgcctgtttggggagcgaagccgtcatgacctggggaacacccaagtaactgtcgatgaacaacaactgcaggtcagtccatttatttatggtgtgcatgcaactacttgtttttctttctcttctttctcttgcttactattttcctccttccttgtttgtctctag

SEQ ID NO: 87 - protein, 993 aa, Oryza minuta
MTETVVSMAMSLVGSAIRVASSASSQEMSMLIGVQNEIWFIKDELKTMQAFLRAAEVRKDQDELVKVWAEQVRDLAYDIEDCLQEFAVHVGHQSLSMQLIKLCHRHRIAVQIRNLKQRVEEVSNRNMRYNLIKSVPSSSTDDSQSNMELIRYQTAHYVNESELVGFDVPKKEILELVSSSENAEAQTIWIVGEGGLGKTTLAKKVYESSDVSSKFPCRAWITVSRSFNVKDLLKNMIKQLLGEDSLKKLLDEHKEVIVEKHNLTDHLNKGLRGRRYFLVLDDLWTTQAWDCIKPTSWGNNMEGSRVVVTTRNKNLAGGSSTSPVYCLKTLEKEDATKLLLRKTKRSLHDIEKDQMKEIFEKILKKCGGLPLAIITIGAVLEGKDIKEWEILYAQLPSELESNPIAEPMKKVVTLSYNYLPSHLKPCFLYLCIFPEDFDIQRKRLVHRWIAEGFIRARGGVGIVDVAQKYFDELINRSMIQASRVDIEGNIKSCRVHDIMRDVMISISREENFVYLMGDDGTSVVEENIRHLVHHDTSKCSNIGMDWSHVRSLTLFGNERPKGLSPSFCFPQLKMLRVLDLQDVKFGMTQKDIGKIGLLRHLKYVNIGGHSSIYALPRCIGKLKDLCTLDITDSYITELPTEISKLQSLCILRCRGRPNSGDFNLNDPKDCLIAFSCLPLLMAATDSDERNKIIAELHVGCSSQWSPNGGTYGVRVPRGIKNLKRLQVLETVDINRTSSKSVEELGELIQLRKLSVVTQGSTKEKCKILCTAIQKLTSLKTLYLNAHGPLDTGTLEWLHSISHLPSLRIIRLIGYMKEMPNWFRELRQLVKIHLQNSQLEEDKTMEILGELPNLMLLFLSWRAVLIIRNQKQLKEVRFEEGTSPWMERIDIRECRLTSGIAGIKHLPRLKEISLEYSAKVVRLGQLEVEMGTHPNRPMLRLFGERSRHDLGNTQVTVDEQQLQVSPFIYGVHATTCFSFSSFSCLLFSSFLVCL

SEQ ID NO: 88 - DNA, 3096 bp, Oryza minuta
  CDS (1)..(3096)
atggcggagacggtgctgagcatggcgaggtcgctggtgggcagtgccatcagcaaggccgcctctgccgctgccaatgagacgagcctcctgctcggcgtcgagaaggacatctggtatatcaaagatgagctaaaaacaatgcaggcattccttagagctgctgaagttatgaaaaagaaagatgaactattaaaggtttgggcagagcaaatacgtgacctgtcgtatgacattgaagattcccttgatgaatttaaagtccatattgaaagccaaaccctatttcgtcagttggtgaaacttagagagcgccaccggatcgctatccgtatccacaacctcaaatcaagagttgaagaagtgagtagcaggaacacacgctacaatttagtcgagcctatttcctccggcacagaggatgacatggattcctatgcagaagacattcgcaatcaatcagctcgaaatgtggatgaagctgagcttgttgggttttctgactccaagaaaaggctgcttgaaatgatcgataccaatgctaatgatggtccggccaaggtaatctgtgttgttgggatgggtggtttaggcaagacagctctttcgaggaagatctttgaaagcgaagaagacattaggaagaacttcccttgcattgcttggattacagtgtcacaatcatttcacaggattgagctacttaaagatatgatacgccaacttcttggccccagttctctggatcaactcttgcaagaattgcaagggaaggtggtggtgcaagtacatcatctttctgagtacctgatagaagagctcaaggagaagaggtactttgttattctagatgatctatggattttacatgattggaattggataaatgaaattgcatttcctaagaacaataagaagggcagtcgaatagtaataaccactcggaatgttgatctagcggagaagtgtgccacagcctcactggtgtaccaccttgatttcttgcagatgaacgatgccataacattgctactgagaaaaacaaataaaaatcatgaagacatggaatcaaataaaaatatgcaaaagatggttgaacgaattgtaaataaatgtggtcgtctaccattagcaatacttacaataggagctgtgcttgcaactaaacatgtgtcagaatgggagaaattctatgaacaacttccttcagaactagaaataaacccaagcctggaagctttgaggagaatggtgaccctaggttacaaccacctaccatcccatctgaaaccatgctttttgtatctaagtatctttcctgaggattttgaaatcaaaaggaatcgtctagtaggtagatggatagcagaagggtttgttagaccaaaggttgggatgacgactaaggatgtcggagaaagttactttaatgagctaatcaaccgaagtatgattcaacgatcaagagtgggcatagcaggaaaaattaagacttgtcgaatccatgatatcatccgtgatatcacagtttcaatctcgagacaggaaaattttgtattattaccaatgggagatggctctgatttagttcaggaaaacactcgccacatagcattccatgggagtatgtcctgcaaaacaggattggattggagcattattcgatcattagctatttttggtgacagacccaagagtctagcacatgcagtttgtctagatcaattgaggatgttacgggtcttggatcttgaagatgtgacattcttaatcactcaaaaagatttcgaccgtattgcattgttgtgccacttgaaatacttgagtattggatattcgtcatccatatattcacttcccagatccattggtaaactacagggcctacaaactttgaacatgctgagaacatacattgcagcactaccaagtgagatcagtaaactccaatgtctgcatactcttcgttgtagtagaaagtttgtttatgacaactttagtctaaaccacccaatgaagtgcataactaacacaatatgcctgcctaaagtattcacacctttagttagtcgcgatgatcgtgcaaaacaaattgctgaattgcacatggccaccaaaagttgctggtctgaatcattcggtgtgaaggtacccaaaggaataggtaagttgcgagacttgcaggttctagagtatgtagatatcaggcggaccagtagtagagcaatcaaagagctggggcacttaagcaagttgaggaaattaggtgtgataacaaaaggctcgacaaaggaaaaatgtaagatactttatgcagccattgagaagctctcttccctccaatctctctatgtgaatgctgcgttattatcagatattgaaacacttgagtgcctagattctatttcatctcctcctcccctactgaggacactcgggttgaatggaagtcttgaagagatgcctaactggattgagcagctcactcacctgaagaagatctacttattgaggagcaaactaaaggaaggtaaaaccatgctgatacttggggcattgcccaacctcatggtcctttatctttattggaatgcttaccttggggagaagctagtattcaaaacgggagcattcccaaatcttagaacacttcgtatttacgaattggatcagctaagagagatgagatttgaggatggcagctcacccctgttggaaaagatagaaatctcttgctgcaggttggaatcagggattattggtatcattcaccttccaaggctcaaggagatttcacttgaatacaaaagtaaagtggctaggcttggtcagctggagggagaagtgaacacacacccaaatcgccccgtgctgcgaatggacagtgaccgaagggatcacgacctgggggctgaagccgaaggatcttctatagaagtgcaaacagcagatcctgttcctgatgccgaaggatcagtcactgtagcagtggaagcaacggatccccttcccgagcaggagggagagagctcgcagtcgcaggtgatcacgttgacgacgaacgataggtcagtcactccctacatggcagcttaa

SEQ ID NO: 89 - protein, 1031 aa, Oryza minuta
MAETVLSMARSLVGSAISKAASAAANETSLLLGVEKDIWYIKDELKTMQAFLRAAEVMKKKDELLKVWAEQIRDLSYDIEDSLDEFKVHIESQTLFRQLVKLRERHRIAIRIHNLKSRVEEVSSRNTRYNLVEPISSGTEDDMDSYAEDIRNQSARNVDEAELVGFSDSKKRLLEMIDTNANDGPAKVICVVGMGGLGKTALSRKIFESEEDIRKNFPCIAWITVSQSFHRIELLKDMIRQLLGPSSLDQLLQELQGKVVVQVHHLSEYLIEELKEKRYFVILDDLWILHDWNWINEIAFPKNNKKGSRIVITTRNVDLAEKCATASLVYHLDFLQMNDAITLLLRKTNKNHEDMESNKNMQKMVERIVNKCGRLPLAILTIGAVLATKHVSEWEKFYEQLPSELEINPSLEALRRMVTLGYNHLPSHLKPCFLYLSIFPEDFEIKRNRLVGRWIAEGFVRPKVGMTTKDVGESYFNELINRSMIQRSRVGIAGKIKTCRIHDIIRDITVSISRQENFVLLPMGDGSDLVQENTRHIAFHGSMSCKTGLDWSIIRSLAIFGDRPKSLAHAVCLDQLRMLRVLDLEDVTFLITQKDFDRIALLCHLKYLSIGYSSSIYSLPRSIGKLQGLQTLNMLRTYIAALPSEISKLQCLHTLRCSRKFVYDNFSLNHPMKCITNTICLPKVFTPLVSRDDRAKQIAELHMATKSCWSESFGVKVPKGIGKLRDLQVLEYVDIRRTSSRAIKELGHLSKLRKLGVITKGSTKEKCKILYAAIEKLSSLQSLYVNAALLSDIETLECLDSISSPPPLLRTLGLNGSLEEMPNWIEQLTHLKKIYLLRSKLKEGKTMLILGALPNLMVLYLYWNAYLGEKLVFKTGAFPNLRTLRIYELDQLREMRFEDGSSPLLEKIEISCCRLESGIIGIIHLPRLKEISLEYKSKVARLGQLEGEVNTHPNRPVLRMDSDRRDHDLGAEAEGSSIEVQTADPVPDAEGSVTVAVEATDPLPEQEGESSQSQVITLTTNDRSVTPYMAA

SEQ ID NO: 90 - DNA, 3220 bp, Oryza minuta
atggcggatacagtactcagcattgcaaagtccctggtgggaagtgctgtaagcaaggttgcttcggttgccgcagacaagatgatcatgctgctgggagtgcagaaggagatatggttcatcaaagatgagctacaaacgatacaagcatttttgattgctgccgaagcatcaaagaaaagcatactattgaaggtttgggtgcagcaagtaagggatctttcctatgacattgaagattgccttgatgaatttacagttcatgtgggcagccaaaacttgtcgaggcagttgatgaagctaaaggatcgccatcggattgccatccagatccgcaatctcaggacaagaattgaagaagtaagcactaggaacatacgctacaacttaatagagaatgacctcacctgcaccactactgatgagaggaatttatttatggaagacattcgcaaccaataagctaacaacattgaggaagctgatcttgtgggtttttctggacccaaaagagagttgcttgatcttatagatgtccatgccaatgacggacctacaaaagttgtatgtgttgtcggtatgggtggtttgggtaagactactattgcaaggaaaatttatgaaagcaaagaggacattgcaaagaatttttcttgctgtgcttggattactgtttcacagtcctttgttagggtggaactactcaaggatttgatggtgaaactttttggagaggaagtactgaagaagcggctgagagaactcgaagggaaggttccacaagtagacgacctcgccagctacctcaggacagagttaaatgaaaggaggtactttgttgtgcttgataacgtgtggagtacagattcatggaaatggattaatagtattgccttccctagaaataacaataaagggagccgggtgatagtaacaacaagagatgttggcttaactaaggagtgtacttctgaattgcttatctaccagcttaaacccctagaaataaactatgcaaaagagttgcttctacggaaagcaaataaagcaataggagatatggaaagtgataaaaagatgagtgacattataactaaaataataaagaagtgtggctatttaccgctggctatactcacaataggaggcgtgctttccaccaaagagataagagagtgggaaactttttatagtcagataccttcagagcttgagagcaacccaaaccttgaagcaatgagaaggatagtgaccctaagttacaactacttaccgtctcatcttaagcaatgctttttgtatctaagcatatttcctgaggattttgaaattaataggaaccgtctggtaaatagatggatggcagaggggtttattaaagctaggactaatatgactattgaagatgttgggaaaagttactttaaagaacttatcaaccgtagcatgattcagccatcaagagcgggtatacgaggagattttaagagctgtcgagtccacgacatcatgcgtgatattacaatttcgatttctagagaagaaaatttcacactcttacccgatggcactgactatgatgtagtacatgggaacactcggcacatagaatttcacgggagtaagtattgctctgaaacaagcttggactggagcattatacggtcattaactatgtttggtgagaggtccgtagaactagagcattcagtttgttcatctcagttgaggatgttacgggtcttggatctaacagatgcacaattttctatcacacaaaatgatgtcgacaacatagtgctcttgtgccacttgaaatacctatgcattaagatacagataccgttcaccatatatttattcacttccacaatccatagctagactgcatggtctgcagacattggacttgggtcagacgtacatttcaacactgccaactcagattactaacttcggagtctccgtagccttcgatgcatgaaagaatatttttcttcttctttaacaacatatttaactaacacattatgcctgcccatgatattcacacctttcgttagtacctcggatcgttctgaaacaattgctaaattgcacatggccaccaaaggcttccgttcaaaatcaaatggtgtcaaggtaccaaaaggaatatgtaataaaaaaactcgacttacaaatattggaggtagtggatattagaaggactagcagtagagcaatcaaagagttggggcagttaagcaagctgaggaaattatgtgtggtaacaaagggatccacaaaggaaaaatgtgagatactctatacagctatccagaagctctgtttcctacaatctctccatgtgaatgctgtgggattttcaggtattggaacacttcagtgtatagattctatttcatctcctcctcccctactgaggacactcaggttgaatggaagtcttgaggagatgcctaactggattgagcagctcacgcacctgatgaagttcaacttatggaggagcaaactaaaagaaggtaaaaccatgttggtacttgcggcgttgcccaacctcatggtcctttatcttcattccaatgcttaccatggggagaagctagtattcaaaatgggagcattcccaaatcttagaacattttcgatttacaatttggagcagctaagagagattagatttgaggacggcagctcaatcttgttggaaaagatagaaatattcaggggttggaatcagggattgttggtatcattcaccttccaaggctcaaggagatttcacttggatacggaagtaaagtggctaggcttggtcagctggagggagaagtgcgcacacacccaaatcaccccgtgctgcgaatgagggaggaccgaagtgatcacgaccttgcttgtgacgccgaaggatcccctgttgaagtggaagcaacagatcctgtgagagctcacagttgcaggtgatcacgttgacaacgaacgacaggtcagtcactccctacacggcatcttaatgaacttgttttatcctcttgtgagatcgatgattttaactcaccctttcatctctctcgttttcttaacctaacagcgaagagataagcacaacttaagctggtttgatcaagtgatgatctcctcctccattggcatctccggtcgtccctgcttctgcggctgcgcacctcgctgctccgaggaggggtgctga

SEQ ID NO: 91 - protein, 1054 aa, Oryza minuta
MADTVLSIAKSLVGSAVSKVASVAADKMIMLLGVQKEIWFIKDELQTIQAFLIAAEASKKSILLKVWVQQVRDLSYDIEDCLDEFTVHVGSQNLSRQLMKLKDRHRIAIQIRNLRTRIEEVSTRNIRYNLIENDLTCTTTDERNLFMEDIRNQANNIEEADLVGFSGPKRELLDLIDVHANDGPTKVVCVVGMGGLGKTTIARKIYESKEDIAKNFSCCAWITVSQSFVRVELLKDLMVKLFGEEVLKKRLRELEGKVPQVDDLASYLRTELNERRYFVVLDNVWSTDSWKWINSIAFPRRNNNKGSRVIVTTRDVGLTKECTSELLIYQLKPLEINYAKELLLRKANKAIGDMESDKMSDIITKIIKKCGYLPLAILTIGGVLSTKEIREWETFYSQIPSELESNPNLEAMRRIVTLSYNYLPSHLKQCFLYLSIFPEDFEINRNRLVNRWMAEGFIKARTNMTIEDVGKSYFKELINRSMIQPSRAGIRGDFKSCRVHDIMRDITISISREENFTLLPDGTDYDVVHGNTRHIEFHGSKYCSETSLDWSIIRSLTMFGERSVELEHSVCSSQLRMLRVLDLTDAQFSITQNDVDNIVLLCHLKYLCIKIQIPFTIYLFTSTIHSTAWSADIGLGSDVHFNTANSDYLRSLRSLRCMKEYFSSSLTTTLTNTLCLPMIFTPFVSTSDRSETIAKLHMATKGFRSKSNGVKVPKGICNKKTRLTNIGGSGYKDQSNQRVGAVKQAEEIMCGNKGIHKGKMDTLYSYPEALFPTISPCECCGEFRYWNTSVYRFYFISSSPTEDTQVERKSGDALDAAHAPDEVQLMEEQTKRRNHVGTCGVAQPHGPLSSFQCLPWGEASIQNGSIPKSNIFDLQFGAAKRDIGRQLNLVGKDRNIQGLESGIVGIIHLPRLKEISLGYGSKVARLGQLEGEVRTHPNHPVLRMREDRSDHDLACDAEGSPVEVEATDPVRAHSCRSRQRTTGQSLPTRHLNELVLSSCEIDDFNSPHLSRFLNLTAKRAQLKLVSSDDLLLHWHLRSSLLLRLRTSLLRGGVL

SEQ ID NO: 92 - DNA, 3276 bp, Oryza minuta
  CDS (1)..(3276)
atggcggagacggtgctgagcatggcgaggtcgctggtgggcagcgccatcagcaaggccgcctccgctgctgccgacgagaccagcctcctgctgggcgtcgagaaagacatctggtatatcaaagatgagttgaaaataatgcaggcattccttagagctgcagaagttatgaaaaagaaagacgaactattaaaggtttgggcagagcaaatacgtgacctgttatatgacattgaagattcccttgatgaatttaaggtccatgttgaaagtcaaaccctatttcgtcagtatatcaaagatgagctaaaaacgatgcaagcattccttagagctgctgaacttatgaaaaagaaagatgaactattaaaggtttgggcagagcaaatacgtgacctgtcatatgacattgaagattcccttgatgaatttaaggtccatattgaaagccaaaccctatttcgtcagttggtgaaactcagagaacgccaccgaattgctatccgtatccacaaccttaaatcaagagttgaagaagtgagtagcaggaacacacgctacagtttagtcaagcctatttcctctggcacagagattgacatggattcctatgcagaagacattcgtaatcagtcagctcgcaatgtggatgaagctgagcttgttgggttttctgactccaagaaaaggttgcttgaaatgatcgataccaatgctaatgatggtccggccaaagtaatctgtgttgttgggatgggtggtttaggcaagacagctctttcgaggaagatctttgaaagcgaagaagacattaggaagaacttcccttgcaatgcttggattacagtgtcacaatcatttcacaggattgagctactcaaagatatgatacgccaacttctaggtcccaattctctgaaacaactcttgcaagaattgcaagggaaggtggtggtgcaagtacatcatctttctgagtacctgatagaagagctcaaggagaagaggtactttgttattctagatgatctatggattttacatgattggaattggataaatgaaattgcatttcctaagaacaataagaagggcagtcgaatagtaataaccactcggaatgttgatctagcggagaagtgtgccacagcctcactggtgtaccaccttgatttcttgcagatgaacgatgccataacattgctactaagaaaaacaaataaaaatcatgaagacatggaatcaaataaaaatatgcaaaagatggttgaacgaattgtaaataaatgtggtcgtctaccattagcaatacttacaataggagctgtgcttgcaactaaacatgtgtcagaatgggagaaattctatgaacagcttccttcagaactagaaataaacccaagcctggaagctttgaggagaatggtgaccctaggttacaaccacctaccatcccatctgaaaccatgctttttgtatctaagtatctttcctgaggattttgaaatcaaaaggaatcgtctggtaggtagatggatagcagaagggtttgttagaccgcaggttgggatgacgactaaggatgtcggagaaagttactttaatgagctaatcagccgaagtatgattcaacgatcaagagtgggcatatcaggaaaaattaagacttgtcgaatccatgatatcatccgtgatatcacagtttcaatctcgagacaggaaaattttgtattgttaccaatgggagatggctctgatttagttcaggaaaacactcgccacatagcattccatgggagtatgtcctgcaaaacaggattggattggagcattattcgatcattagctatttttggtgacagacccaagagtctagcacatgcagtttgtctagatcaattgaggatgttacgggtcttggatcttgaagatgtgacattcttaatcactcaaaaagatttcgaccgtattgcattgttgtgccacttgaaatacttgagtattggatattcgtcatccatatattcacttcccagatccattggtaaactacagggcctacaaactttgaacatgctgagaacatacattgcagcactaccaagtgagatcagtaaactccaatgtctgcatactcttcgttgtagtagaaagtttgtttatgacaactttagtctaaaccacccaatgaagtgcataactaacacaatatgcctgcctaaagtattcacacctttagttagtcgcgatgatcgtgcaaaacaaattgctgaattgcacatggccaccaaaagttgctggtctgaatcattcggtgtgaaggtacccaaaggaataggtaagttgcgagacttgcaggttctagagtatgtagatatcaggcggaccagtagtagagcaatcaaagagctggggcacttaagcaagttgaggaaattaggtgtgataacaaaaggctcgacaaaggaaaaatgtaagatactttatgcagccattgagaagctctcttccctccaatctctctatgtgaatgctgcgttattatcagatattgaaacacttgagtgcctagattctatttcatctcctcctcccctactgaggacactcgggttgaatggaagtcttgaagagatgcctaactggattgagcagctcactcacctgaagaagatctacttattgaggagcaaactaaaggaaggtaaaaccatgctgatacttggggcattgcccaacctcatggtcctttatctttattggaatgcttaccttggggagaagctagtattcaaaacgggagcattcccaaatcttagaacacttcgtatttacgaattggatcagctaagagagatgagatttgaggatggcagctcacccctgttggaaaagatagaaatctcttgctgcaggttggaatcagggattattggtatcattcaccttccaaggctcaaggagatttcacttgaatacaaaagtaaagtggctaggcttggtcagctggagggagaagtgaacacacacccaaatcgccccgtgctgcgaatggacagtgaccgaagggatcacgacctgggggctgaagccgaaggatcttctatagaagtgcaaacagcagatcctgttcctgatgccgaaggatcagtcactgtagcagtggaagcaacggatccccttcccgagcaggagggagagagctcgcagtcgcaggtgatcacgttgacgacgaacgatagcgaagagataggcacagctcaagctggctga

SEQ ID NO: 93 - protein, 1091 aa, Oryza minuta
MAETVLSMARSLVGSAISKAASAAADETSLLLGVEKDIWYIKDELKIMQAFLRAAEVMKKKDELLKVWAEQIRDLLYDIEDSLDEFKVHVESQTLFRQYIKDELKTMQAFLRAAELMKKKDELLKVWAEQIRDLSYDIEDSLDEFKVHIESQTLFRQLVKLRERHRIAIRIHNLKSRVEEVSSRNTRYSLVKPISSGTEIDMDSYAEDIRNQSARNVDEAELVGFSDSKKRLLEMIDTNANDGPAKVICVVGMGGLGKTALSRKIFESEEDIRKNFPCNAWITVSQSFHRIELLKDMIRQLLGPNSLKQLLQELQGKVVVQVHHLSEYLIEELKEKRYFVILDDLWILHDWNWINEIAFPKNNKKGSRIVITTRNVDLAEKCATASLVYHLDFLQMNDAITLLLRKTNKNHEDMESNKNMQKMVERIVNKCGRLPLAILTIGAVLATKHVSEWEKFYEQLPSELEINPSLEALRRMVTLGYNHLPSHLKPCFLYLSIFPEDFEIKRNRLVGRWIAEGFVRPQVGMTTKDVGESYFNELISRSMIQRSRVGISGKIKTCRIHDIIRDITVSISRQENFVLLPMGDGSDLVQENTRHIAFHGSMSCKTGLDWSIIRSLAIFGDRPKSLAHAVCLDQLRMLRVLDLEDVTFLITQKDFDRIALLCHLKYLSIGYSSSIYSLPRSIGKLQGLQTLNMLRTYIAALPSEISKLQCLHTLRCSRKFVYDNFSLNHPMKCITNTICLPKVFTPLVSRDDRAKQIAELHMATKSCWSESFGVKVPKGIGKLRDLQVLEYVDIRRTSSRAIKELGHLSKLRKLGVITKGSTKEKCKILYAAIEKLSSLQSLYVNAALLSDIETLECLDSISSPPPLLRTLGLNGSLEEMPNWIEQLTHLKKIYLLRSKLKEGKTMLILGALPNLMVLYLYWNAYLGEKLVFKTGAFPNLRTLRIYELDQLREMRFEDGSSPLLEKIEISCCRLESGIIGIIHLPRLKEISLEYKSKVARLGQLEGEVNTHPNRPVLRMDSDRRDHDLGAEAEGSSIEVQTADPVPDAEGSVTVAVEATDPLPEQEGESSQSQVITLTTNDSEEIGTAQAG

SEQ ID NO: 94 - DNA, 1355 bp, Oryza minuta
  CDS (1)..(1353)
atggcggatacagtactcagcattgcaaagtccctggtgggaagtgctgtaagtaaggttgcttcggttgctgccgacaagatgatcttgctgctgggagtgcagaaggagatatggttcatcaaagacgagctacaaacgatacaagcatttttgatggctgccgaagcatcaaagaaaagcatactgttgaaggtttgggtgcagcaagtaagggatctatcctatgacattgaagattgccttgatgaatttacagttcatgtgggcagccaaaccttgtcgaggcagttgatgaagctaaaggatcgccatcggatcgccgttcagatccgcaatctcaggacaagaatcgaagaagtgagcagtaggaacacacgctacaacttaatagagaatgaccttaccagcaccactggtgagaggaatttcattatggaagacattcgaaatgaatcagctaacaatatcgaggaagctgagcttgtgggtttttcgggacccaaaagagagttgcttgatcttatagatgtccatgccaaggatggacctacaaaggttgtatgtgttgtcggtatgggtggtttgggtaagactactattgcaaggaaaatttatgaaagcaaagaggacattgcaaagaatttttcttgctgtgcttggattactgtttcacagtcctttgttaggttggaactactcaaggatttgatggtgaaactttttggagaggaagtactcaagaagcggctgagagaacttgaagggaaggttccacaagtagacgacctcgccagctacctcaggacagagttaaatgaaaggaggtactttgttgtgcttgatgatgtgtggagtacagattcatggaaatggattaatagtattgccttccctagaaataacaataaagggagccgggtgatagtaacaacaagagatgttggcttagctaaggagtgtacttctgaattgcttatctaccggcttaaacccctagaaataacctatgcgaaagagttgcttctaaggaaagcaaataaaaaaattgaagatatgaaaagtgataaaaagatgagtgacatcattactaaaatagtaaaaaagtgtggctatttaccactggctatactcacaataggaggcgtgcttgccaccaaagatccatcaagagcgggtgtacgaggagaatttaagagctgtcgaatccacgacatcatgcgtgatattgcaatttcgatttctagagaggaaaatttcatactcttacctgagggcactgactatgacgtagtgcatgggaacactcggcacatagcatttcacgggagtaagtattgctctgaaacaagctt

SEQ ID NO: 95 - protein, 451 aa, Oryza minuta
MADTVLSIAKSLVGSAVSKVASVAADKMILLLGVQKEIWFIKDELQTIQAFLMAAEASKKSILLKVWVQQVRDLSYDIEDCLDEFTVHVGSQTLSRQLMKLKDRHRIAVQIRNLRTRIEEVSSRNTRYNLIENDLTSTTGERNFIMEDIRNESANNIEEAELVGFSGPKRELLDLIDVHAKDGPTKVVCVVGMGGLGKTTIARKIYESKEDIAKNFSCCAWITVSQSFVRLELLKDLMVKLFGEEVLKKRLRELEGKVPQVDDLASYLRTELNERRYFVVLDDVWSTDSWKWINSIAFPRNNNKGSRVIVTTRDVGLAKECTSELLIYRLKPLEITYAKELLLRKANKKIEDMKSDKKMSDIITKIVKKCGYLPLAILTIGGVLATKDPSRAGVRGEFKSCRIHDIMRDIAISISREENFILLPEGTDYDVVHGNTRHIAFHGSKYCSETS

SEQ ID NO: 96 - DNA, 2265 bp, Oryza minuta
tacgccagctctaatacgactcactatagggcaaagctggtacgcctgcaggtaccggtccggaattcccgggtcgacccacgcgtccgaacaaattgctgaattgcacatggccaccaaaagttgctggtctgaatcattcggtgtgaaggtacccaaaggaataggtaagttgcgagacttgcaggttctagagtatgtagatatcaggcggaccagtagtagagcaatcaaagagctggggcacttaagcaagttgaggaaattaggtgtgataacaaaaggctcgacaaaggaaaaatgtaagatactttatgcagccattgagaagctctcttccctccaatctctctatgtgaatgctgcgttattatcagatattgaaacacttgagtgcctagattctatttcatctcctcctcccctactgaggacactcgggttgaatggaagtcttgaagagatgcctaactggattgagcagctcactcacctgaagaagatctacttattgaggagcaaactaaaggaaggtaaaaccatgctgatacttggggcattgcccaacctcatggtcctttatctttattggaatgcttaccttggggagaagctagtattcaaaacgggagcatttccaaaatcttagaacacttcgtatttacgaattggatcagctaagagagatgagatttgaggatggcagctcacccctgttggaaaagatagaaatctcttgctgcaggttggaatcagggattattggtatcattcaccttccaaggctcaaggagatttcacttgaatacaaaagtaaagtggctaggcttggtcagctggagggagaagtgaacacacacccaaatcgccccgtgctgcgaatggacagtgaccgaagggatcacgacctgggggctgaagccgaaggatcttctatagaagtgcaaacagcagatcctgttcctgatgccgaaggatcagtcactgtagcagtggaagcaacggatccccttcccgagcaggagggagagagctcgcagtcgcaggtgatcacgttgacgacgaacgatagcgaagagataggcacagctcaagctggctgacgatctcctcccccatcagcgtcgtcatcagcgaacagatagggcagggcttccctgcttctgcgtgcacctcactgctctgactctgagggacatgatgatcaatgaggcttccagttcccaaatgtgcgcctaacacactcagtcattcccatccaagagagtttgctagggtggagatgcggtcatgtggatgatcaaatggactacatcagacgcatcacactgctgcccaacctttacgcactgttgacaaatggagtgcaggtcctaaaccaggccagaagtttgttcagtgttcatgttcccaaacatacattctggatggcagaatgacttgctctcaatggttgaagctgcaatcttccaggctcatttctggctgaacccaaattggtggcttggaggctggagctgcatgacatcagagatcacaatggccactttttgttgccatgggtgagggtacatggatcatgccgctgagcctttactccgagctagcagaaaactggctggctgtagatcgagttcgccatcgccctcaactttgtcgatgtgatggtgatcatggatagatgtatgtcacagcgcaaagcggagactccaatgttcgcaaccatactgtaaatgtgggaggggaaaatttctccaaggggaccaaccatggaacagaggaagatggagcaaagtagttgctcttgctatcatttcagagctcagggctgatctctagctcagataggagtactaattcttatgtgtgtgtatttgatctatgatggtacgttttaagagttggaccagatattgcttgatctttacatgatgaattgatgatatgcaaaaaatggtatttgacatggagacgcaggaacagggtggttcattgatctgtttgatcttaatcttggagagctaggatggaaaactgagttggtggttatatctctactacttttgatgttgtccaaaactaatttgtttctactcatgtacgtatgtaaaaaggaaaattttaatgattaaagaacttattgttctctttaatcagattatttatcagtgggatttttctttctctggtctgtataaaaatactttttttatcgtcaatcctcctaaaattcctatttataaaaaaaaaaaaaaagggcggccg

SEQ ID NO: 97 - DNA, 2351 bp, Oryza minuta
gatcaaatgaaagaaatatttgaaaagatactaaagaaatgtggaggtctaccgctagctataatcacaataggtgcagtccttgaagggaaagatataaaagagtgggaaattttgtatgctcaacttccatcagaacttgaaagcaatccaatcgctgaaccaatgaagaaggtggtaacccttagttacaattacttgccatctcatcttaagccttgctttctgtacctttgcatctttcctgaggattttgatatccaaaggaagcgcctagtacatagatggattgcagaggggtttattagagctaggggtggagtgggaattgtggatgtggcacaaaagtattttgatgagttgatcaaccgaagtatgattcaagcatctagagtggatatagagggaaatattaagagctgccgagtccatgatatcatgcgtgatgtcatgatatcaatatctagggaagaaaattttgtatacttgatgggtgatgatggaactagtgtagtggaggaaaatattcgccatttagtgcaccatgatactagcaagtgttcaaatataggcatggattggagccatgtacggtcattaactttgtttggcaatgagagacccaaagggctatctccttcattttgttttccccaattgaagatgctaagagtgctggatcttcaagatgtcaaatttgggatgacacaaaaagatatcggaaaaatagggttgctgcgtcacttgaaatatgtgaatattggagggcattcaagtatttatgcacttcctaggtgtataggaaaattaaaagacttgtgcactttggacataactgacagttacattacagaactaccaactgagattagtaaattgcagagtctatgtattctccgttgtagaggaagaccaaactcgggggattttaatctaaatgatcctaaggattgtctaattgccttctcatgtttgcctctgcttatggctgcaaccgattctgatgaacgtaataaaataattgctgagctacacgtgggttgttcaagtcaatggtctcctaatggtggtacatatggtgtgagggtacctagaggaatcaagaatttgaaaaggctgcaggtgctagagacagtggatatcaatcgaaccagcagtaagtcagttgaagagttgggagagcttatccagctaagaaaactaagtgtagtaacacaagggtccaccaaggaaaaatgcaaaatactctgtacagccattcagaagctaacttccctcaaaactctctatctgaatgctcatggacccttggatactggaacacttgaatggctacattctatttcccatcttccttccctgaggatcatcagattgatcggatacatgaaagagatgcccaactggtttagggagctcagacagctggtgaagattcacttgcagaatagccaactagaggaggataaaacaatggagatactcggagaactgcctaatctcatgctccttttccttagttggcgagcgtatgctggggggaagctggtgttcagggagggaacattctaaaatctcagggtgctaatcattcgcaatcagaagcaactgaaagaggtgagatttgaagagggaacctcaccctggatggaaaggattgatatcagagaatgcagattgacatcagggattgctggtatcaaacaccttccgaggcttaaggagatttcacttgaatacagtgctaaagtggtgaggctaggtcagctagaggttgaaatgggcacacaccccaatcgacccatgttgcgcctgtttggggagcgaagccgtcatgacctggggaacacccaagtaactgtcgatgaacaacaactgcagccaaaccaaacagttagcgaagaatgcttgatggatgccgatcttgcttctagatgatgatgatcagcttcctgttgcaagctctcctggtgatcgttcttcttcagttcttgacctaccaccaatgctggctagcttcctgtagtgctgatctcggagtcccctgctgaattttgcacctccggcgccgagaggccagttcactgtacatcccccctgagacacaatgtcggtatttgtttgattggatatgatttgattaagtacgatgatttttggttctgaaaatttgtatcatgattggttggagcgtgtgattaggatgcttatatttgcagcagaggtcgatcgtattttgttttgctgtgcacacaaaatgttcgtcccaaatgtcttgtcagatattttgtagcttttgattawmaarwamwmmrwawwwawrwwwcgtaatatgaaaaaaaaaaaaaaaa

(19) United States
(12) Patent Application Publication
(10) Pub. No.: US 2004/0006788 A1
(43) Pub. Date: Jan. 8, 2004

(54) Procedures and materials for conferring disease resistance in plants

(76) Inventors: Guo-Liang Wang, Columbus, OH (US); Guifu Liu, Beijing (CN)

Correspondence Address: CALFEE HALTER & GRISWOLD, LLP, 800 SUPERIOR AVENUE, SUITE 1400, CLEVELAND, OH, 44114, US

(21) Appl. No.: 10/352,179

(22) Filed: Jan. 27, 2003

Related U.S. Application Data
(60) Provisional application No. 60/352,106, filed on Jan. 25, 2002.
(60) Provisional application No. 60/353,304, filed on Feb. 1, 2002.

(51) Int. Cl. A01H 1/00; C12N 15/82; C07H 21/04; C12N 9/10; C12N 5/04; A01H 5/00

(52) U.S. Cl. 800/279; 800/320.2; 435/69.1; 435/193; 435/320.1; 435/419; 536/23.2

(57) ABSTRACT

The present invention is in the field of rice genetics. More specifically, the invention relates to nucleic acid molecules from regions in the rice genome, which are associated with resistance to the fungal pathogen Magnaporthe grisea. The invention relates to methods which employ such nucleic acid molecules to produce plants, particularly rice plants, that are resistant to infection by Magnaporthe grisea. The invention relates to the use of such nucleic acids or fragments thereof as markers for resistance to infection with Magnaporthe grisea in a plant breeding program. The invention also relates to proteins encoded by such nucleic acid molecules as well as antibodies capable of recognizing these proteins.

This application claims priority under 35 USC 119 from U.S. Provisional Application Serial No. 60/352,106, filed on Jan. 25, 2002 and U.S. Provisional Application Serial No. 60/353,304, filed on Feb. 1, 2002, the disclosures of which are incorporated herein in their entirety.

### BACKGROUND OF THE INVENTION

Rice blast, caused by the fungus Magnaporthe grisea, is one of the most devastating diseases in rice. The disease occurs in most rice growing areas worldwide, costing farmers a loss of nearly $5 billion per year (Moffat 1994). The high variability in M. grisea's pathogenicity makes the control and management of rice blast difficult. In addition, resistance in many cultivars is short-lived in disease-conducive environments. For the last four decades, rice geneticists and breeders have studied the genetics of blast resistance and have tried to collect new sources of resistant germplasm to breed for durably resistant cultivars.

Genetic analysis of resistance to blast began in the early 1960s when Goto established the differential system for races of M. grisea in Japan (On 1985). Over 20 loci for complete resistance have been mapped relative to molecular markers on the rice molecular map (McCouch et al. 1994). To elucidate the molecular mechanism(s) of blast resistance, map-based cloning of a number of blast resistance genes is being actively pursued in several laboratories. Recently, two resistance genes, Pib and Pita, have been successfully isolated. Pib was introgressed independently from two Indonesian and two Malaysian cultivars into various japonica cultivars (Yokoo et al. 1978). The deduced amino acid sequence of the Pib gene contains a nucleotide binding site (NBS) and leucine-rich repeats (LRRs) (Wang et al. 1999), a common feature of many cloned plant resistance genes (Bent 1996). Interestingly, Pita also encodes a putative cytoplasmic receptor with a centrally localized nucleotide-binding site and leucine-rich domain (LRD) at the C-terminus. AVR-Pita(176) protein is shown to bind specifically to the LRD of the Pi-ta protein, both in the yeast two-hybrid system and in an in vitro binding assay, indicating that the AVR-Pita(176) protein binds directly to the Pi-ta LRD region inside the plant cell to initiate a Pi-α-mediated defense response (Bryan et al. 2000 and Jia et al. 2000). Comparison of the sequences of 6 resistant and 5 susceptible alleles of Pita has revealed overall amino acid polymorphism with only one single amino acid determining specificity.

It is desirable to have additional methods and tools for producing and identifying plants that are resistant to fungal diseases, particularly diseases caused by the fungus Magnaporthe grise.

### SUMMARY OF THE INVENTION

The present invention provides isolated nucleic acids that are useful for producing or identifying plants, particularly plants in the grass family with resistance to diseases caused by the fungus Magnaporthe grisea. In one aspect, the nucleic acids comprise a sequence which encodes the NBS1 protein, the NBS2 protein, the NBS3 protein, the NBS4 protein, the NBS 5 protein, the NBS 6 protein or combinations thereof. Preferably, such sequence is incorporated into a transgene or expression construct which can be used to produce transgenic plants whose genome comprises such sequence. In another aspect, the nucleic acid is a probe that comprises a sequence which specifically hybridizes to a contiguous sequence of at least 15 nucleotides in one or more of the following sequences SEQ ID NO. 84, SEQ ID NO. 86, SEQ ID NO. 88, SEQ ID NO. 90, SEQ ID NO 92, or SEQ ID NO. 94, or the complement thereof. In another aspect, the invention is a primer set which comprises a forward primer and a reverse primer that can be used in a polymerase chain reaction to amplify a unique region in the Pi9 locus. Preferably, the forward and reverse primer comprise a sequence which is identical to or the reverse complement of a contiguous sequence of at least 15 nucleotides in one or more of SEQ ID NO. 84, SEQ ID NO. 86, SEQ ID NO. 88, SEQ ID NO. 90, SEQ ID NO. 92, or SEQ ID NO. 94.

The present invention also provide for methods which employ the present nucleic acids to provide a transgenic plant that is resistant to an infection, particular rice blast, that is caused by the fungus Magnaporthe grisea. The present invention also provides for plants and parts of the plants produced by such method. Plant parts, without limitation, include seed, endosperm, ovule and pollen. In a particularly preferred embodiment of the present invention, the plant part is a seed.

The present invention also provides methods for identifying transgenic or non-transgenic plants that comprise an NBS1, NBS2, NBS3, N1BS4, NBS5, or NBS6 rice blast resistant allele. The method comprises isolating DNA or RNA from a cell of the plant and assaying for the presence of such allele using the primers or probes of the present invention.

### BRIEF DESCRIPTION OF THE DRAWINGS

FIG. 1. SCAR markers pB8 and pBA14 were linked to the Pi9 gene. About 20 ng of genomic DNA and pB8- and pBA14-specific primers were used in the two PCR reactions.

FIG. 2. Southern blot analysis of pB8 (A) and 19L (B) in an F₂ population. About 2 μg of DNA per sample was digested with restriction endonuclease HindIII (in both A and B) and separated on a 1% agarose gel by electrophoresis. PCR products of pB8 and 19L were used as hybridization probes.

FIG. 3. Genetic and physical map of the Pi9 region. The genetic map was constructed using the mapping data from 1280 F₂ plants. The numbers in brackets are genetic distances in centiMorgans from Pi9. Insert size is shown in parenthesis after each BAC clone. Recombinants for each marker were determined in 596 plants using Southern hybridization. The putative Pi9 introgression region was estimated based on the hybridization results of pB8 and 19L that did did not hybridize with IR31917 but did hybridize with both 75-1-127 and O. minuta.

FIG. 4. The genetic linkage map of the Pi2 and Pi9 region. This map is constructed based on the consensus mapping data from DH, Pi9 and Pi2 mapping populations. The numbers at the right side are genetic distances in centiMorgans. RG64 is a RFLP marker from Cornell University. R2123 is a RFLP marker from the Japanese Rice Genome Project. The rest of the markers were isolated from this work. The computer program Mapmaker 3.0 with kosambi function was used in the map construction. FIG. 5. Southern blot hybridization of 75-1-127 (lane 1), IR31917 (lane 2), CO₃₉ (lane 3) and two recombinants (R198-10R, lane 4, and R174-11R, lane 5) with BAC end 12L. DNA was digested with both DraI and HindIII, and separated by electrophoresis on a 1% agarose gel. The membrane was hybridized with a ³²P-labeled 12L probe. Arrow indicates the segregating band among the parents and two recombinants.

FIG. 6. Genomic DNA sequence at the Pi9 locus as well as sequences of the NBS1-NBS6 open reading frames.

FIG. 7. Structure of the Pi9 locus. All the six NBS genes were predicted by the programs including gene prediction and homology search. The string of the sequence is indicated by the gray line (labeled as: specific sequence”). The exons are indicated by the dark boxes along the gray line, and the introns by the white boxes along the line. The arrows show the transcription direction of the NBS genes. The numbers below the NBS genes shows the start and stop site along the sequence string. NBS4 may be a pseudogene, which has some stop codons in the coding region whose exons ar not given. The insertion element indicated in NBS6 shows 94% nucleotide sequence similarity to the LTR of the rice retrotransposon RIRE8.

FIG. 8. Sequence comparison analysis of the six NBS genes. The six putative NBS/LRR genes were translated into proteins. The sequence comparison program of Gap was used to compare the amino sequence of the six putative NBS/LRR proteins and PIB, a cloned blast resistance protein in rice.

FIG. 9. Phylogeny of six NBS genes was conducted using the program of the Phylodendronn.

FIG. 10. Disease reaction of the Pi(transgenic plants.

FIG. 11. PCR analysis of Pi9 mutant lines.

FIG. 12. cDNA sequences for NBS3 and NBS5.

### DETAILED DESCRIPTION OF THE INVENTION

The present invention relates to nucleic acids that can be used to provide and to identify plants, particularly plants in the grass family, more particularly rice plants, that are resistant to disease caused by the fungal pathogen Magnaporthe grisea. The present invention also relates to methods of producing transgenic plants that are resistant to such disease and to methods of identifying plants whose genome comprises one or more NBS rice blast resistant alleles. The nucleic acids and methods of the present invention are based, at least in part, on applicants' discovery of certain genes in the P19 locus that confer resistance to rice blast, a disease caused by the fungus Magnaporthe grisea.

Pi9 confers resistance to 43 isolates collected from 14 countries.

The wild rice Oryza minuta is a tetraploid species with a genomic composition of BBCC and is a source of resistance to both bacterial blight and rice blast (Sitch et al. 1989). Through wild hybridization and repeated backcrossing, the resistance gene Pi9 was transferred from Oryza minuta into the elite breeding line IR31917 (Amante-Bordeos et al. 1992). The introgression line, 75-1-127, was tested for resistance to many Philippine isolates at the International Rice Research Institute (IRR1) and no compatible isolates were identified (H. Leung, personal communication).

A total of 43 rice blast isolates of Magnaporthe grisea collected from 14 countries were used to determine if Pi9 confers resistance to rice blast. To test the spectrum of the Pi9 gene, both Pi9 introgression line 75-1-127 and the susceptible recurrent cultivar, IR31917, were inoculated with the same set of isolates in the inoculation experiment (Table 1). 75-1-127 was highly resistant to all 43 isolates. These inoculation results suggest that Pi9 confers high and broad spectrum resistance to blast.

Three RAPD Markers Linked to Pi9 were Identified.

The RAPD technique (Williams et al. 1990) and bulk segregant analysis (Michelmore et al. 1991) were used to screen for Pi9-linked markers. An F₂ population was generated from a cross between 75-1-127 and the susceptible cultivar, CO₃₉. Seventy-nine F₂ plants were inoculated with Philippine blast isolate PO₆-6. Fifty-four resistant plants and twenty-five susceptible plants were identified which is consistent with a single dominant gene segregation ratio (3:1). DNA was extracted from 10 resistant and 10 susceptible plants.

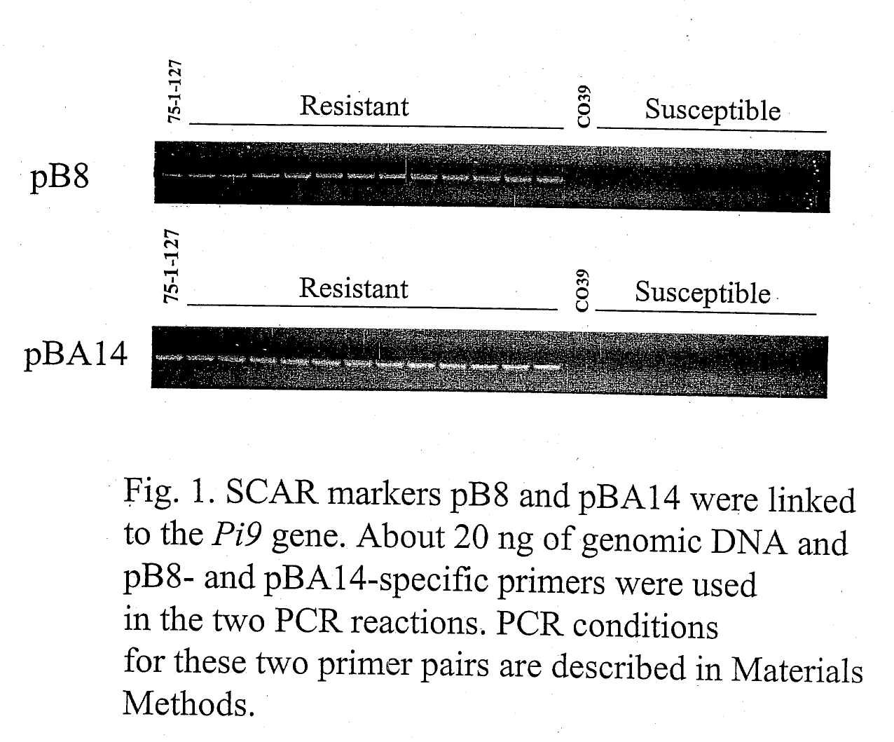

To detect more polymorphic bands and better separation of the amplified fragments, especially those between 100 bp and 1000 bp, ³³P labeled dCTP nucleotide was added to the PCR reaction and the reaction mix was run on a 4.5% polyacrylamide sequencing gel. After screening with over 900 Operon random primers, only three primers were found with reproducible polymorphic bands between the resistant and susceptible pools. After confirmation in 54 individual resistant and 25 susceptible F₂ plants, specific bands were excised from the dried polyacrylamide gel. The eluted DNA was used as a template to re-amplify the band with the same random primer. After gel purification, all three fragments were cloned into the pGEM-T vector (Promega) and the corresponding recombinant clones were named pB8, pBA14 and pBV14. After sequencing the insert, specific primers were designed for each marker based on their sequences. Two markers (pB8 and pBA14) were then successfully converted into SCAR markers since a single band (700 bp for pB8 and 400 bp for pBA14) was amplified in 75-1-127 but not in CO₃₉ (FIG. 1). No polymorphic band was found between resistant plant 75-1-127 and the susceptible line CO₃₉ using primer pairs based on the sequence of marker pBV14, thus, pBV14 was not used in the succeeding experiment.

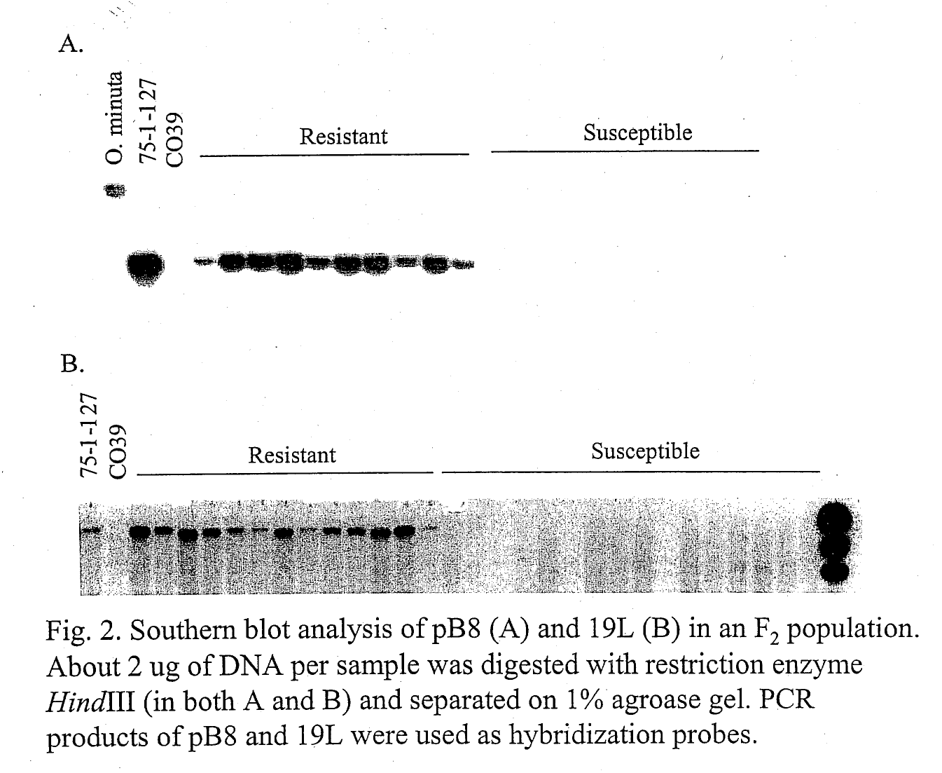

To determine the genetic distance accurately between these two SCAR markers and Pi9, 1280 F2 plants were inoculated with isolate PO₆-6 and the disease reaction of each plant was obtained. Mini-scale DNA was isolated from each plant for PCR amplification. Five of the 1280 F₂ plants showed recombination between pBA14 and Pi9, indicating that the pBA14 marker lies approximately 0.4 cM from Pi9. No recombinant plants between pB8 and Pi9 were identified. Southern hybridization of 75-1-127, CO₃₉ and the O. minuta donor line (accession 101141) genomic DNA revealed that pB8 is a single copy fragment present only in the resistant plant 75-1-127 and O. minuta (FIG. 2A). pBA14 is a medium repetitive element and only a faint polymorphic band was observed between 75-1-127 and CO₃₉, which prevented its use in Southern hybridization analysis of resistant and susceptible plants (data not shown). To confirm the PCR result, DNA was extracted from, 79F₂, 55 F₃ and 462 F₅ plants for Southern analysis. When pB8 was used as a probe, all HindIII digested DNA isolated from the resistant plants showed a single band and no hybridization was detected in any of the susceptible plants (FIG. 2A). Both PCR and Southern hybridization results suggested that pB8 might be a part of or tightly linked to the Pi9 gene.

Construction of a Bacterial Artificial Chromosome (BAC) Library of the Pi9-Introgression Line.

A BAC library was constructed using high molecular weight (HMW) DNA isolated from 75-1-127 according to the procedure described by Wang et al. (1995). Since only one size-selection was performed, the average insert size was about 45 kb. To maximize the chance of getting a DNA fragment containing the Pi9 gene, over 200,000 clones were collected and stored in 100 pools (about 2000 individual clones per pool). The library equaled approximately 21 genome equivalents based on the rice genome size (430 Mb) and average insert size of the BAC clones (45 kb). BAC DNA was mini-prepared from each pool.

For PCR screening of the BAC library, BAC DNA was diluted 5 times to 20/111. For Southern hybridization, 5 μl of BAC DNA (500 ng) was digested with HindIII and separated on 1.0% agarose gel before transferring to nylon membranes. Since the fine-mapping result indicated that pB8 might lie within the Pi9 gene or tightly linked to the gene, the pB8 primer pairs were used to screen the 100 BAC pools. A 700 bp fragment was amplified in 12 of the 100 pools. These 12 positive pools were then confirmed with membrane hybridization using pB8 as the probe. To identify the individual pB8-containing clones, 4000-5000 clones were streaked on three large petri dishes from positive pool. Individual clones that hybridized with the pB8 probe were then isolated. Each positive clone was further confirmed by Southern hybridization. The insert sizes of all 12 identified clones were determined using Bio-Rad's Gene Mapper II after digestion with restriction enzyme NotI. The insert sizes were ranged from 15 kb to 80 kb.

Isolation of BAC Ends for the Construction of a BAC Contig of the Introgression Region.

To construct a contiguous map of BAC clones covering the Pi9 region, two BAC clones (BAC19 and BAC12) were selected because of their insert size (over 50 kb) and different HindIII-restriction digestion patterns. A 600 bp fragment, 12R, at the right end of BAC12 (sp6 side of the BAC vector) was isolated after digestion with both NotI and EcoRI. 12L, a 2.3 kb fragment at the left end of BAC12, was isolated when digested with both NotI and SpeI. For BAC19, the left end consists of the NotI and EcoRV fragment (4.0 kb, 19L) while the right end consist of the NotI and SpeI fragment (3.0 kb, named 19R). To confirm whether or not the two BACs overlap, the isolated end sequences from each BAC (the NotI and HindIII vector sequence was removed) were hybridized with BAC12 and BAC19. Both 12L and 19R hybridized only with their original BAC clones. 12R and 19L ends hybridized to both BAC19 and BAC12, suggesting that these two BACs overlap and extend in opposite directions. This is consistent with the restriction digestion patterns that they had 5 identical bands when digested with XbaI (data not shown). The end sequences from two BAC clones were further characterized for copy number by Southern hybridization. All four ends isolated from the two BACs showed single or few bands in 75-1-127, indicating these ends are suitable for BAC contig construction and chromosome walking.

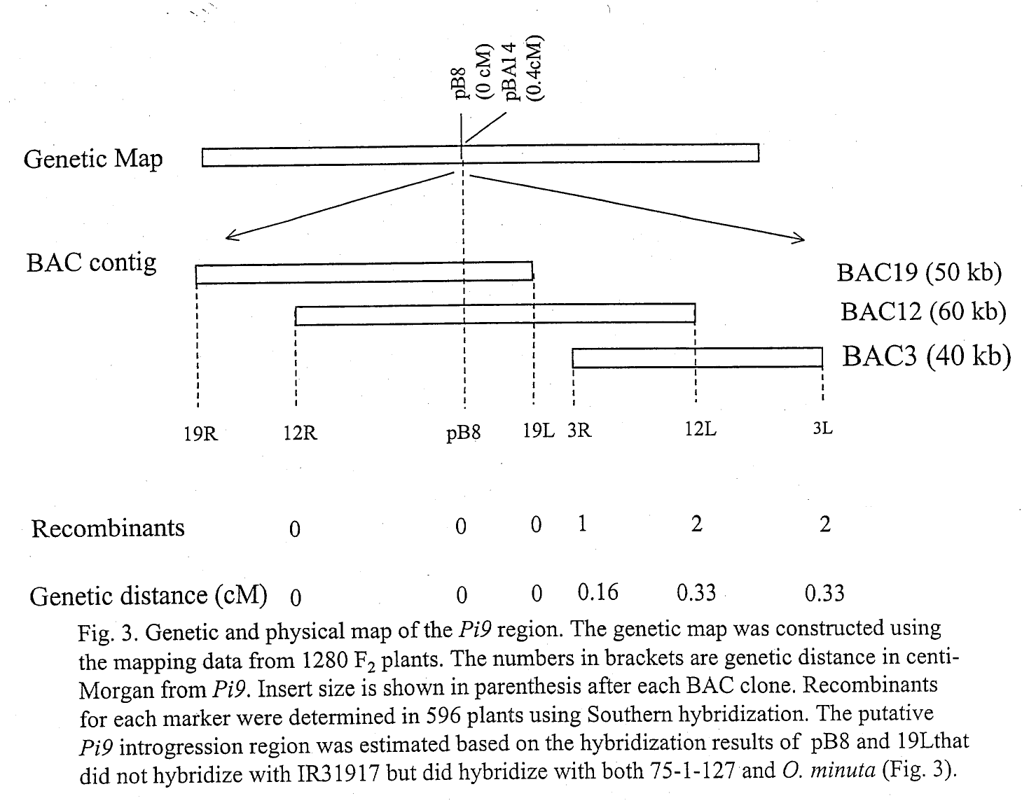

To extend the BAC contig past the 12L end, 12L was used as probe in hybridization with the membranes containing all of the BAC pool DNA. Five BAC pools were identified containing the 12L sequence. Among them, BAC3 (a 40 kb BAC) was chosen for further characterization because of its minimum overlapping with BAC12 based on restriction enzyme digestion patterns (data not shown). Both BAC3 end sequences were obtained using the same method used for the isolation of the BAC12 and BAC19 ends. The BAC3 right end sequence is 3.8 kb and left end sequence is 2.4 kb. Southern hybridization confirmed that 3R overlaps with BAC12 but not with BAC19. Both 3R and 3L ends hybridized with both 75-1-127 and IR31917 (data not shown). From this information, a 100 kb BAC contig comprised of BAC19, BAC12 and BAC3 was constructed (FIG. 3).

Pi9 is Located on Chromosome 6 Between RFLP Marker RG64 and R2131.

To map the Pi9-linked markers and BAC ends on the rice molecular linkage map, a doubled haploid (DH) mapping population derived from a cross between IR64 and Azucena (Huang et al. 1994) was used. The genomic DNA of IR64 and Azucena was first digested with 8 restriction enzymes (BamHI, BglII, DraI, EcoRI, HindIII, PstI and XbaI). When pB8 was used as a probe, no hybridization band was observed although the blot was exposed to X-ray film for 5 days. Another marker, pBA14, contained repetitive sequences and showed no polymorphism between the two mapping parents. Therefore, neither marker is preferred for mapping in the population.

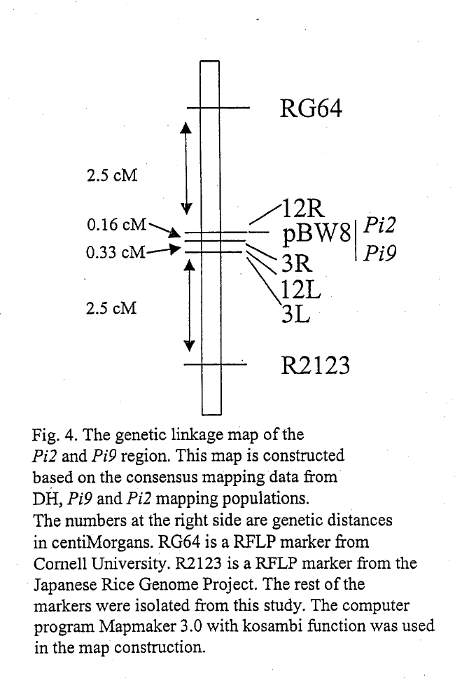

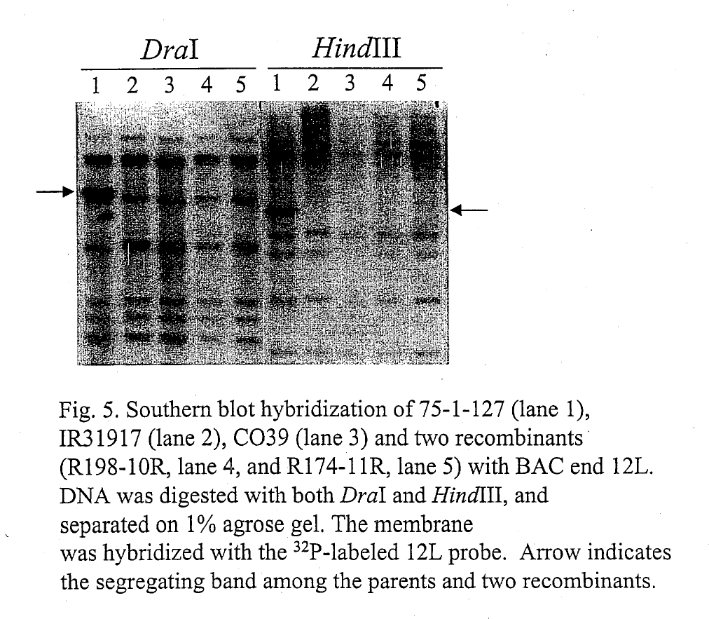

To map the Pi9-linked BAC ends on the linkage map, a parental polymorphism survey was conducted using 18 different restriction enzymes for all 6 BAC ends. Polymorphism between the two mapping parents was detected with only 4 ends (12R, 12L, 3R and 3L) for at least one restriction enzyme. No polymorphism was detected at the 19R locus. Like pB8, no hybridization signal was found when 19L was used as a probe. An appropriate enzyme was selected to digest the 111 DH lines for each BAC end. Mapping data analysis indicated that all 4 sequences were mapped onto chromosome 6 between RFLP markers RG64 and R2131 (FIG. 4).

Establishing a High-Resolution Map at the Pi9 Locus.

To construct a high-resolution map at the Pi9 locus, a total of 596 plants (79 F₂, 55 F₃ and 462 F₅ plants) were used in hybridization with one RAPD marker (pB8) and six BAC ends (12L, 12R, 19L, 19R, 3L and 3R). Among them, 340 plants were resistant and 256 plants were susceptible. Each marker was hybridized with the parental survey blots that contained 75-1-127 and CO₃₉ DNA digested with 14 enzymes. An appropriate enzyme showing polymorphism between the two parents for each marker was used to digest all 596 plants. End 19R was not used in the experiment since it did not show any polymorphism between 75-1-127 and CO₃₉ for 20 enzymes, again confirming that it may be outside the introgression region. Hybridization results indicated that no recombinants were found between Pi9 and either 12R, 19L, or pB8. For 3R, only one recombinant (plant R198-10R) was identified. Two recombinants (R198-10R and R174-11R, resistant phenotype and susceptible genotype) were found when end sequences 12L and 3L were used as probes. The hybridization result of 12L with the two recombinants is shown in FIG. 5. Based on these mapping results, the Pi9 gene was mapped between the BAC ends 19R and 3R (FIG. 3 and FIG. 4).

Sequence Analysis of a 76 kb Fragment at the Pi9 Locus

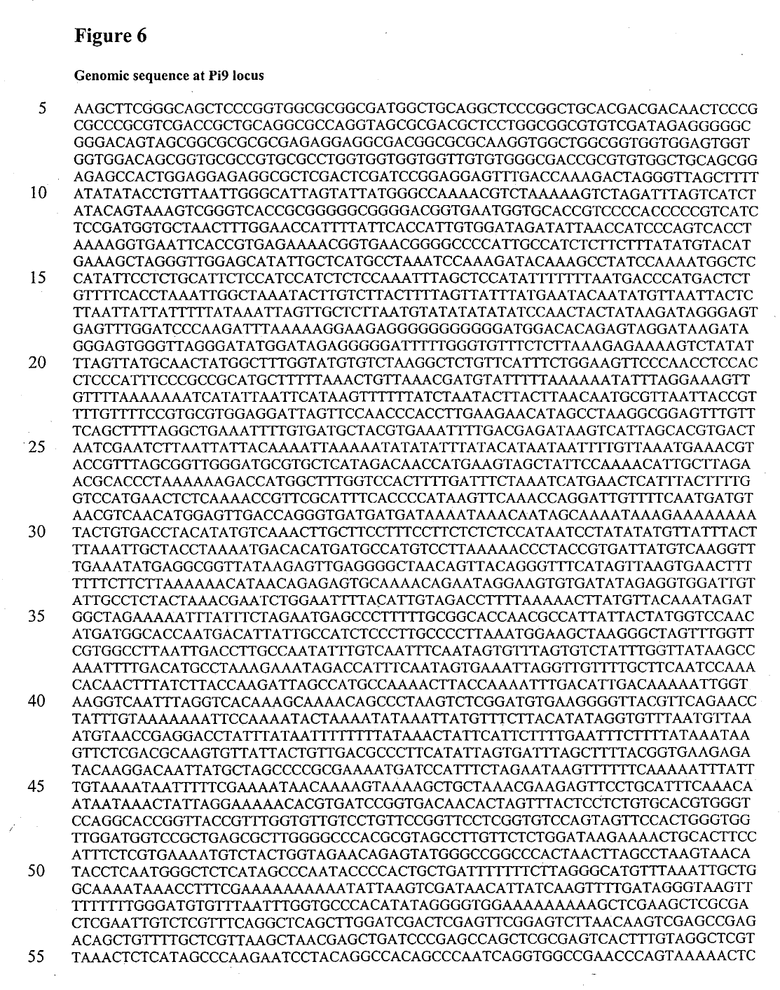

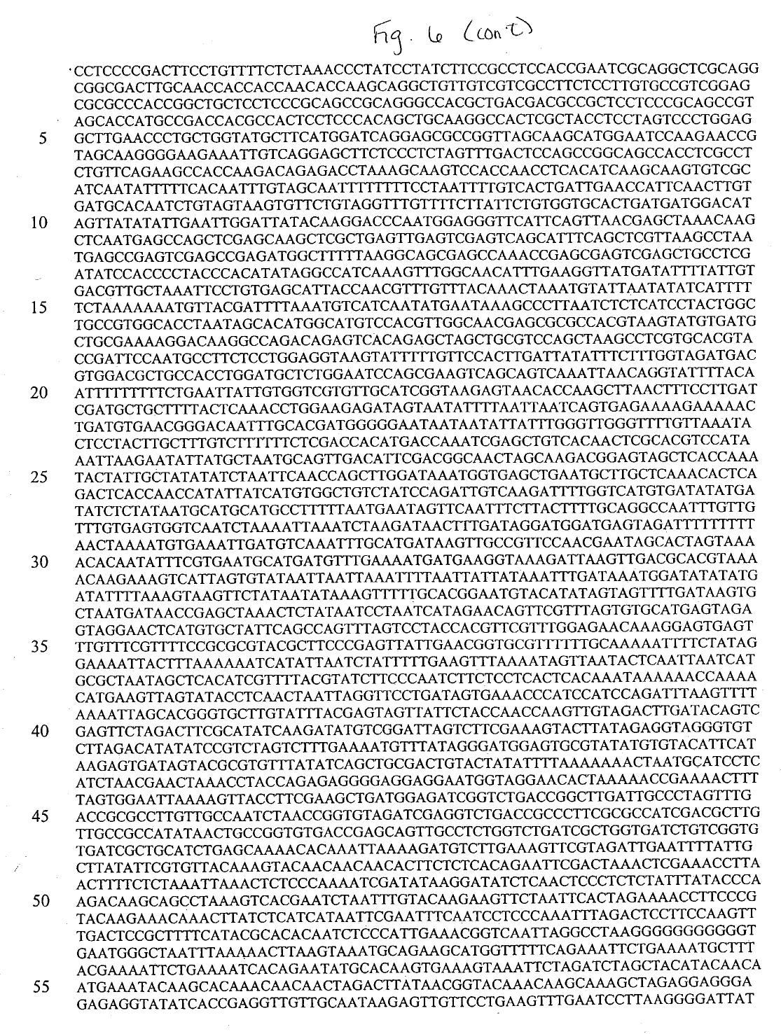

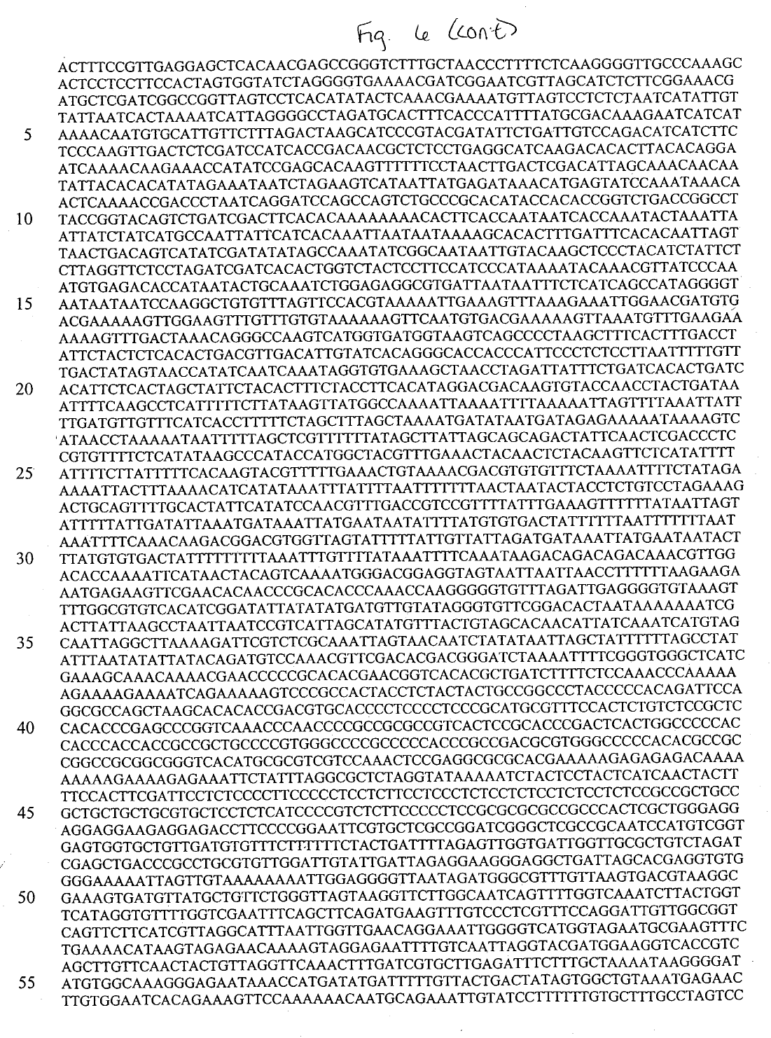

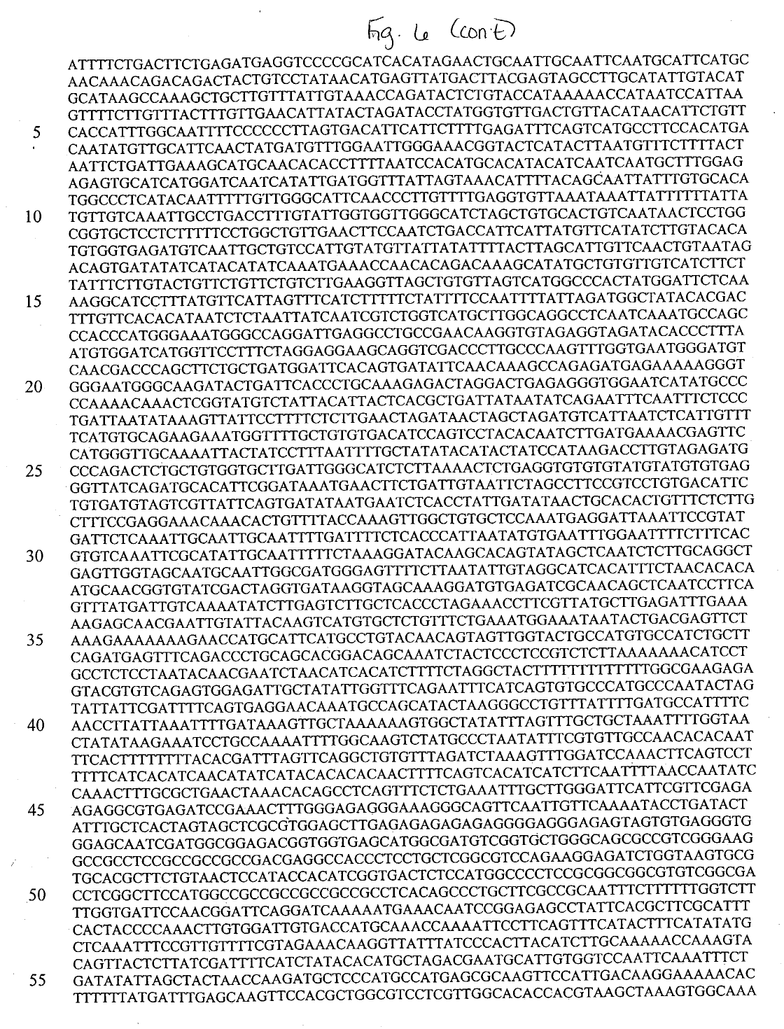

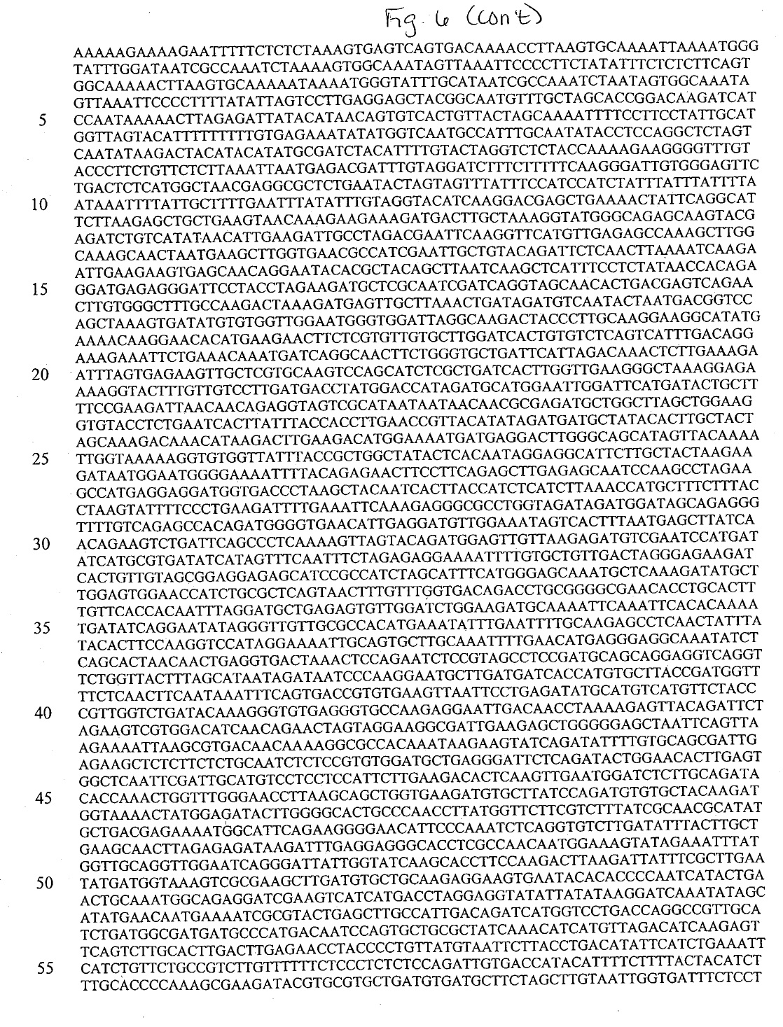

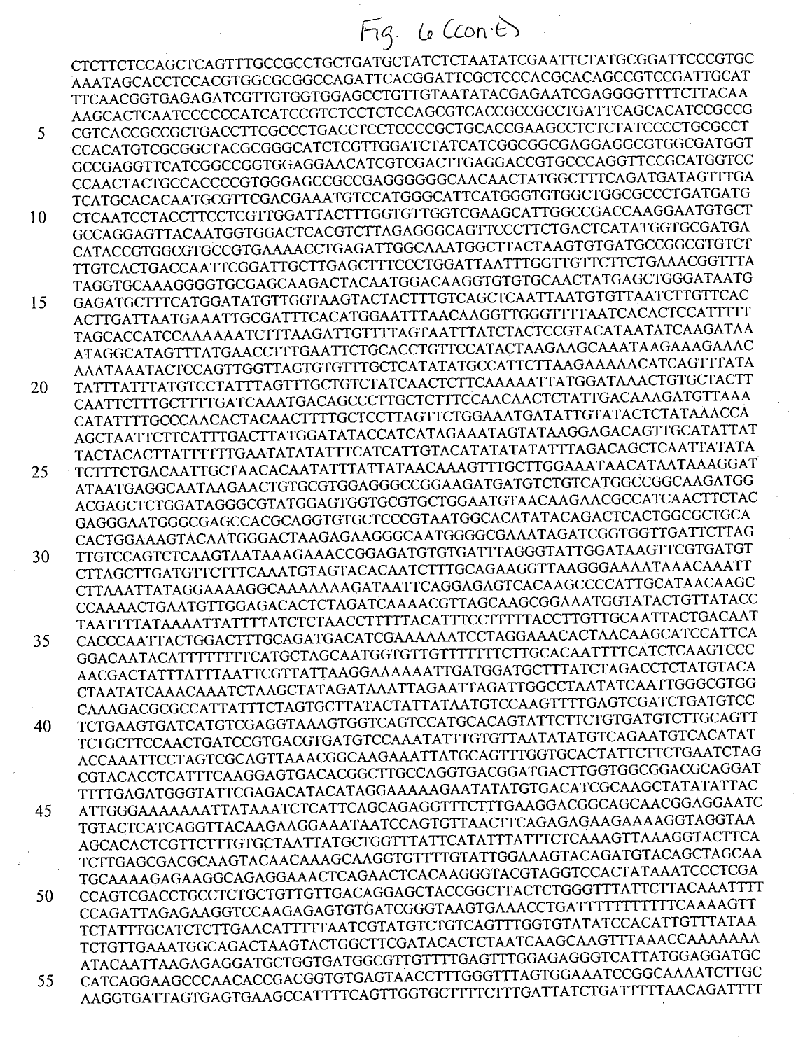

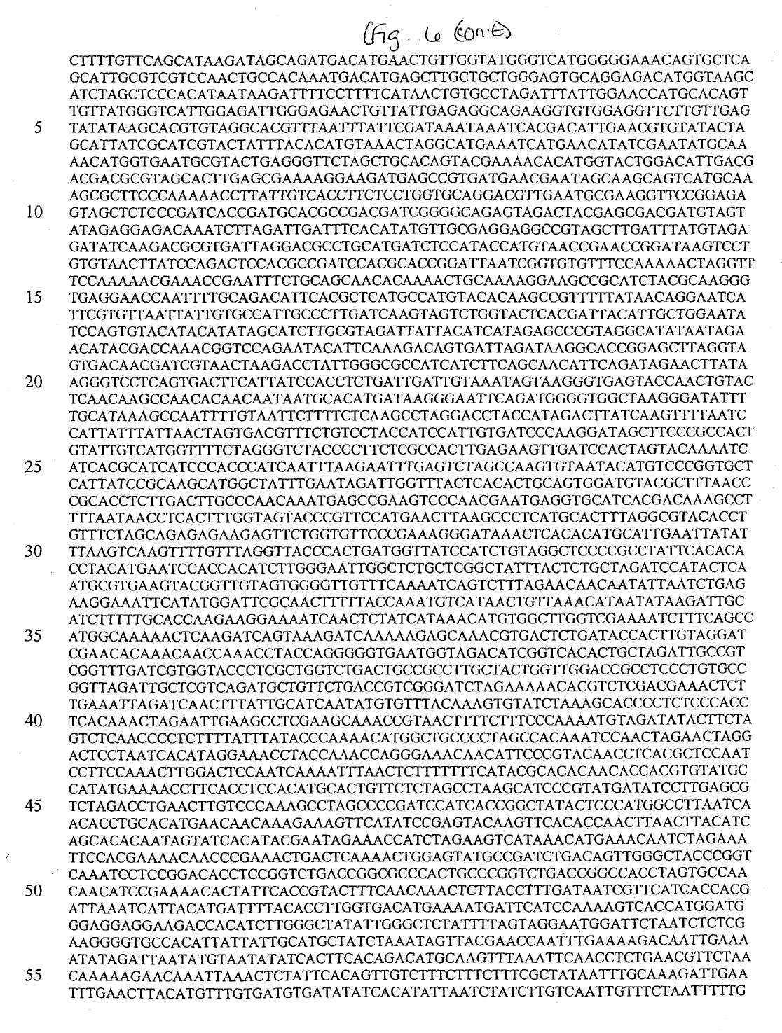

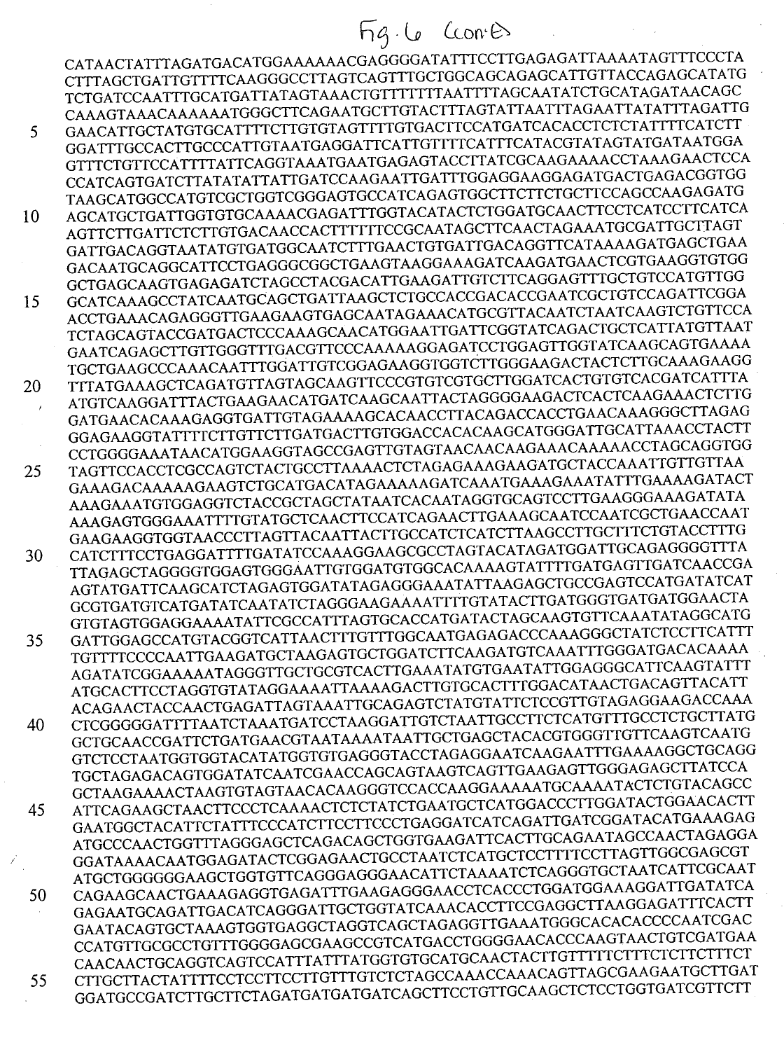

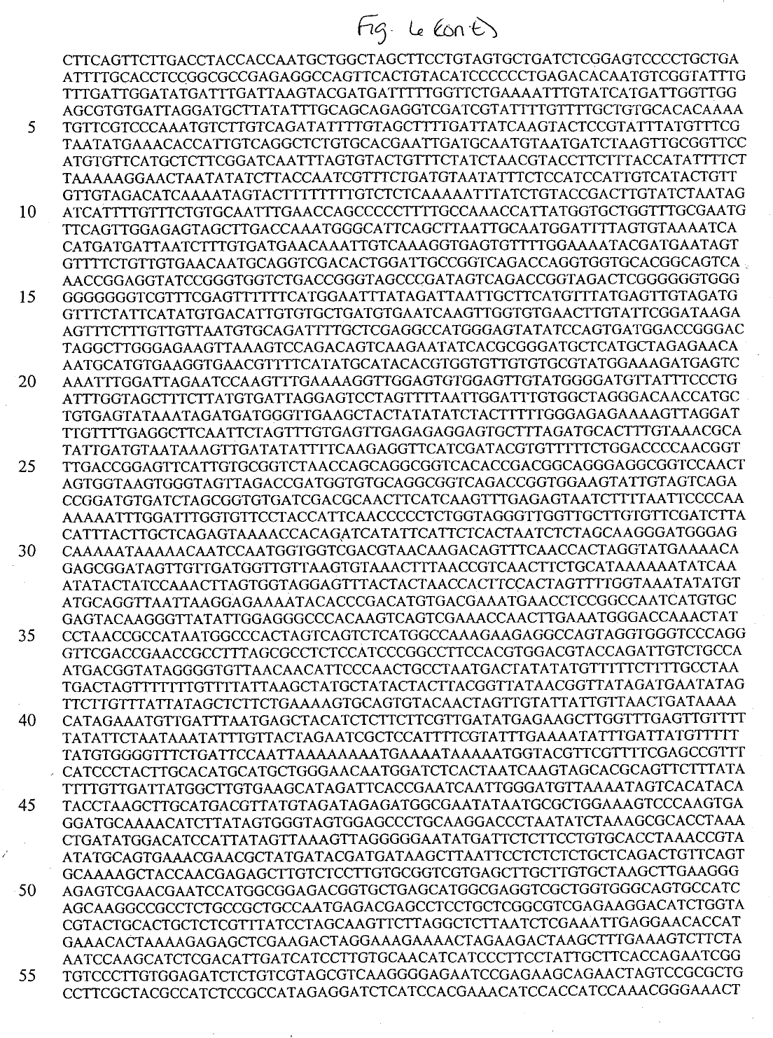

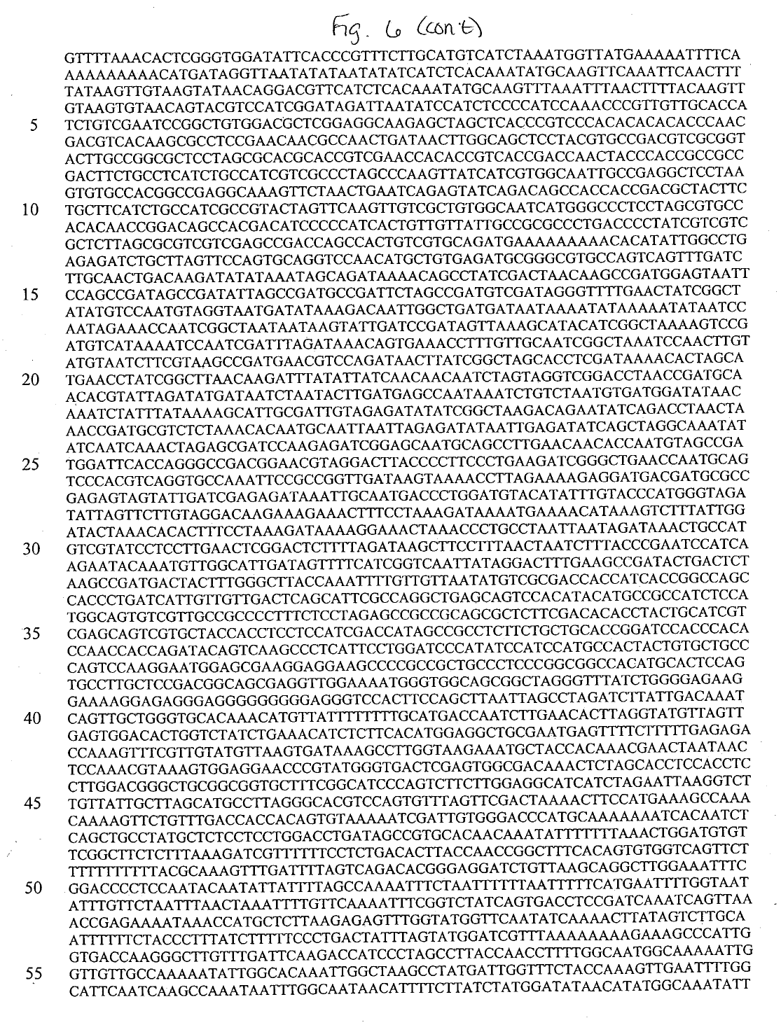

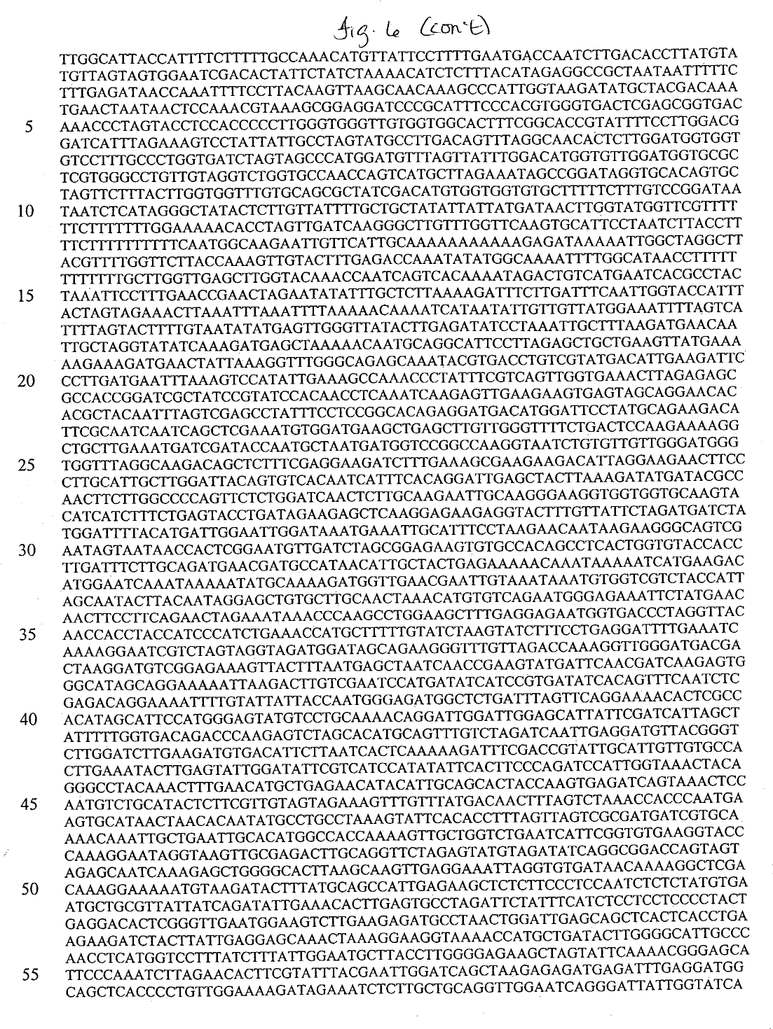

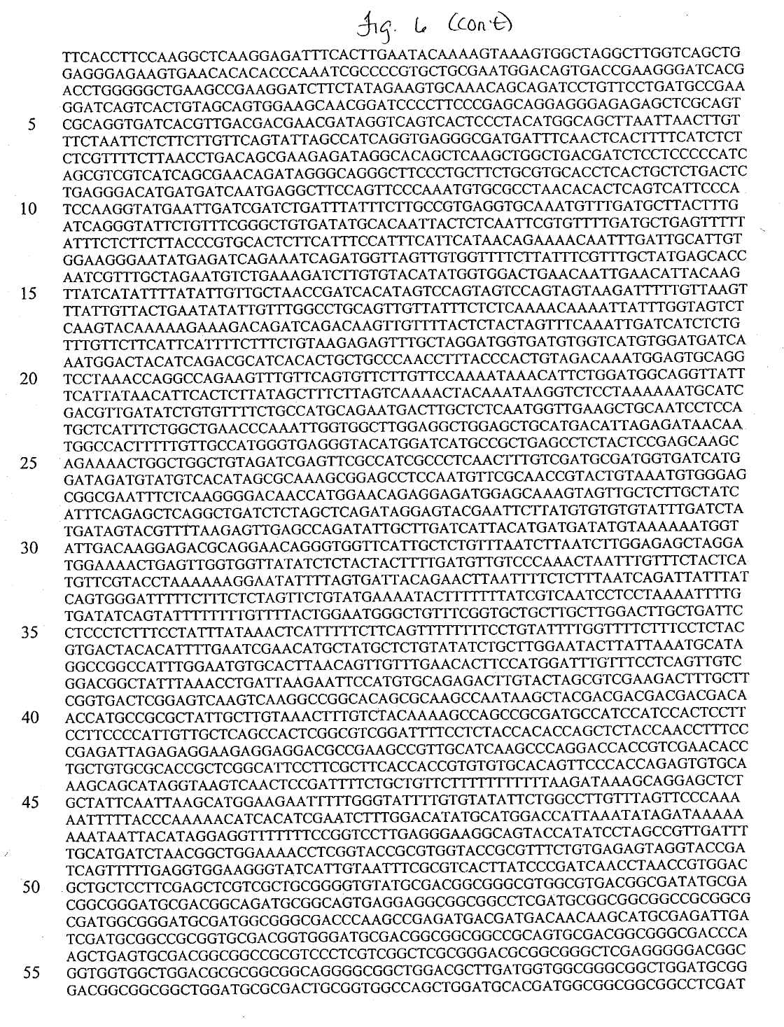

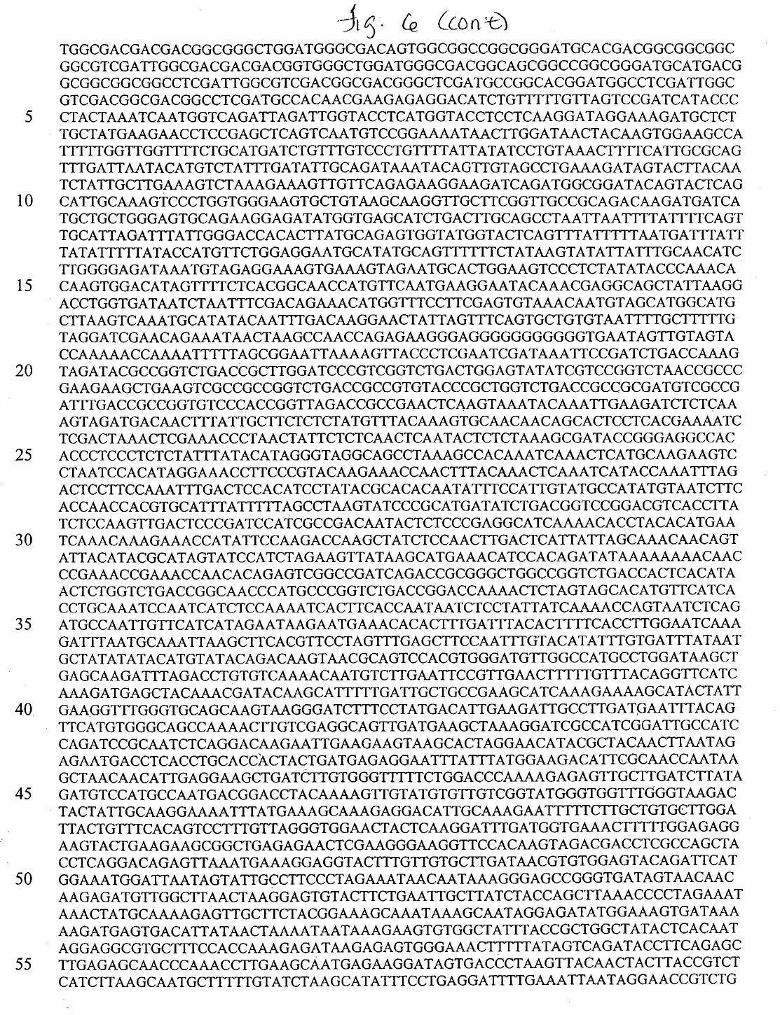

From the hybridization results, it was confirmed that BAC12 and BAC3 overlaps (FIG. 3). To obtain the sequence information at the Pi9 locus, both BAC12 and BAC3 were fully sequenced using a short gun method. Purified plasmids of the two BACs were sonicated, separately, using a sonicator. Sheared DNA fragment was then size-selected on a agarose gel, damaged DNA ends were repaired using a T4 polymerase (Roche), and ligated to a pBluescript (KS) vector. About 700 individual clones from the BAC12 shotgun library and 450 individual clones from the BAC3 shotgun library were sequenced from both ends. The sequence analysis program Phred/Phrap was used to assemble all sequence data. Sequence analysis showed BAC12 is 58 kb and BAC3 is 40 kb. About 18 kb was overlapped between these two BACs. The total length of the DNA fragment from the two BAC is 76,272 bp (FIG. 6).

Identification of a NBS/LRR Gene Cluster

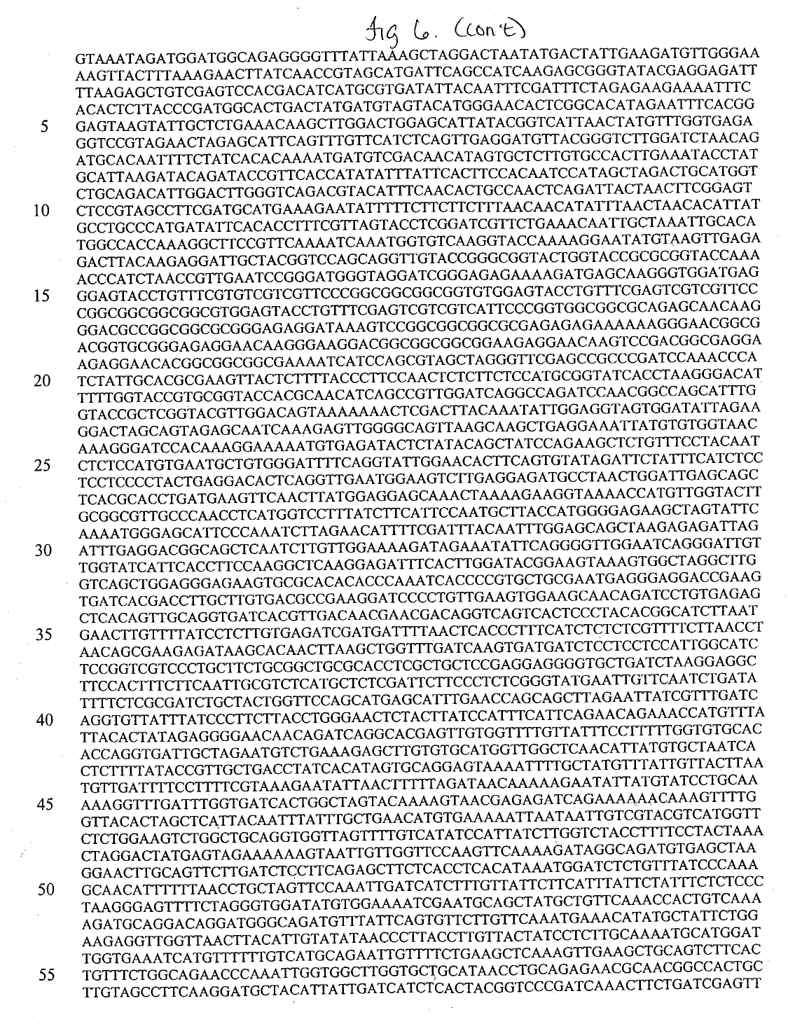

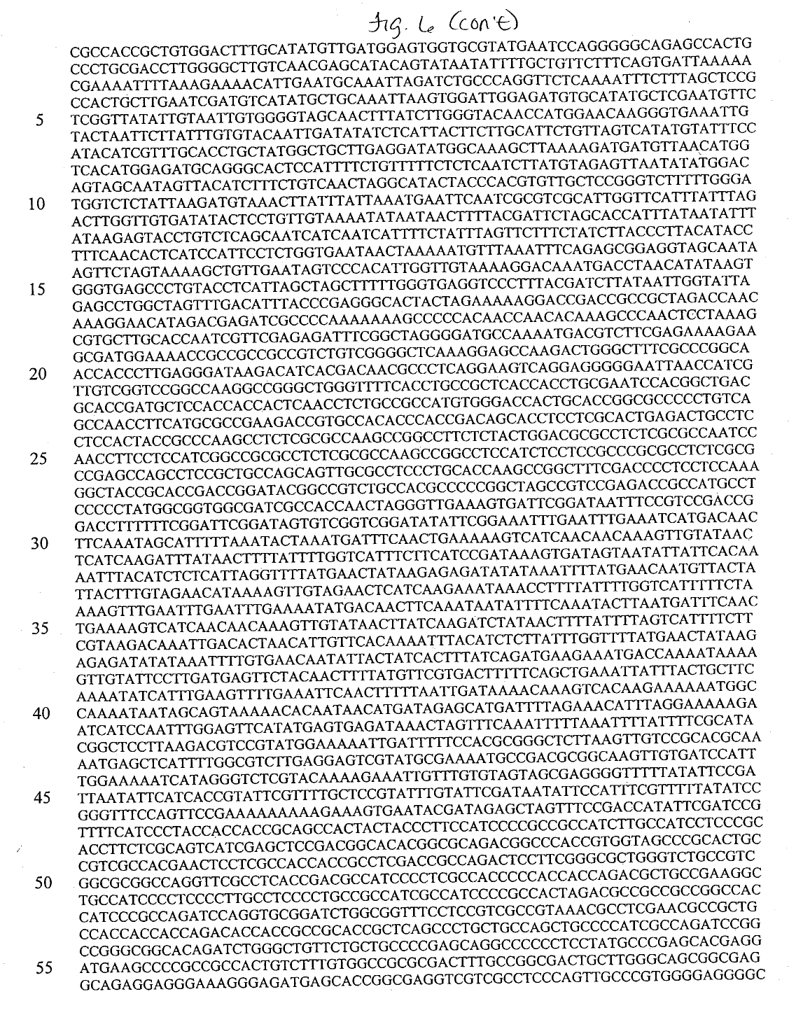

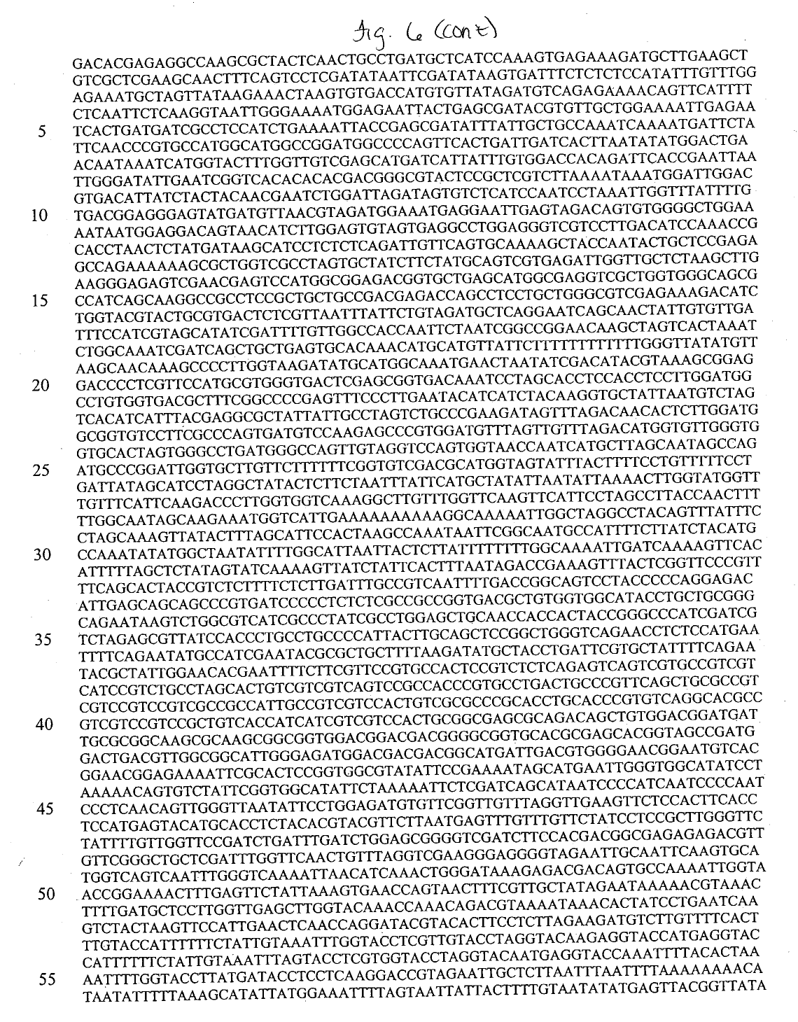

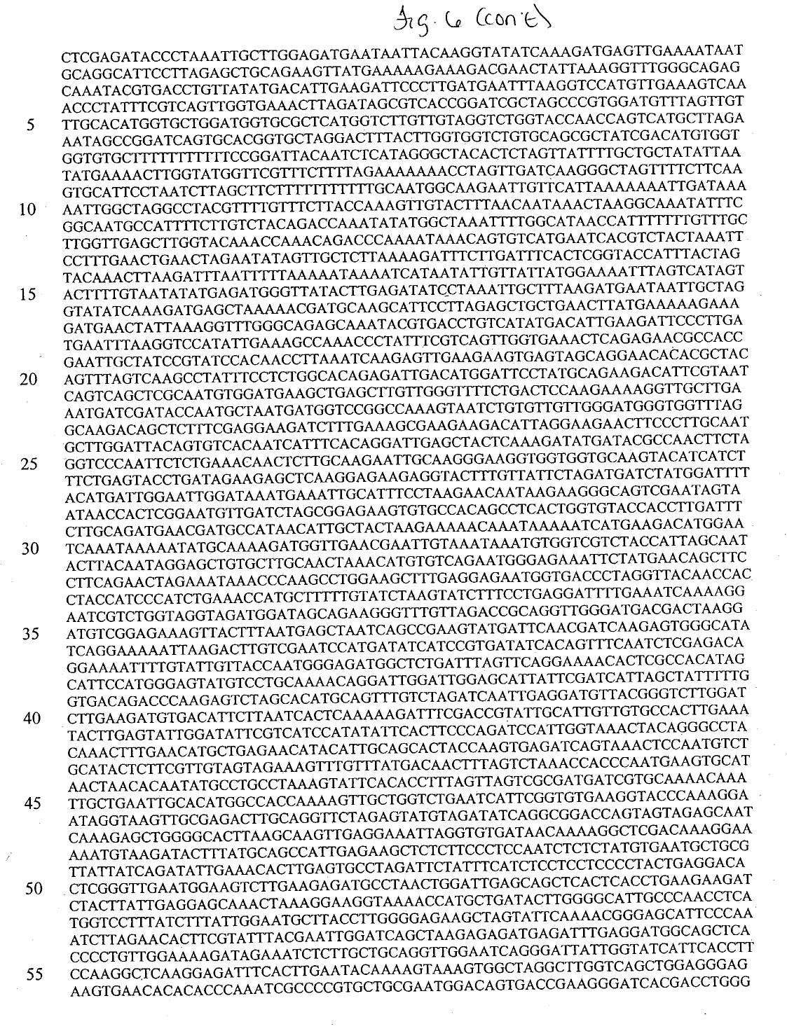

To identify the open reading frame (ORF) accurately from the genomic sequence, two different approaches were used. Firstly, the gene prediction program of GenScan1.0 was used to analyze the coding sequence (CDS) in the 76 kb region. Secondly, the homology search using BLAST program was used to modify the gene prediction result. A total of seven putative genes were identified. The first gene from the sp6 end of BAC12 is homologous to maize nitric induced gene. The other six genes (named NBS1-NBS6) are candidate genes of Pi9 since all of them show high homology to NBS/LRR type disease resistance genes cloned in plant species (FIG. 7, Bent, 1996). The exact position of each NBS/LRR gene is shown in FIG. 7. Among the six Pi9 candidate genes, NBS3 and NBS2 were confirmed with the partial sequence of the relative cDNA. It seems the NBS6 is not complete. This gene is also truncated in the 5′ region by an insertion of a solo-LTR, which shows 94% of identity in nucleotide sequence to the LTR of rice gypsy-type retrotransposon, RIRE8. This solo-LTR shows typical feature including duplicated target sequence of GACCG and inverted sequence of TGTCAC.

Sequence Comparison Analysis of Six Candidate Genes

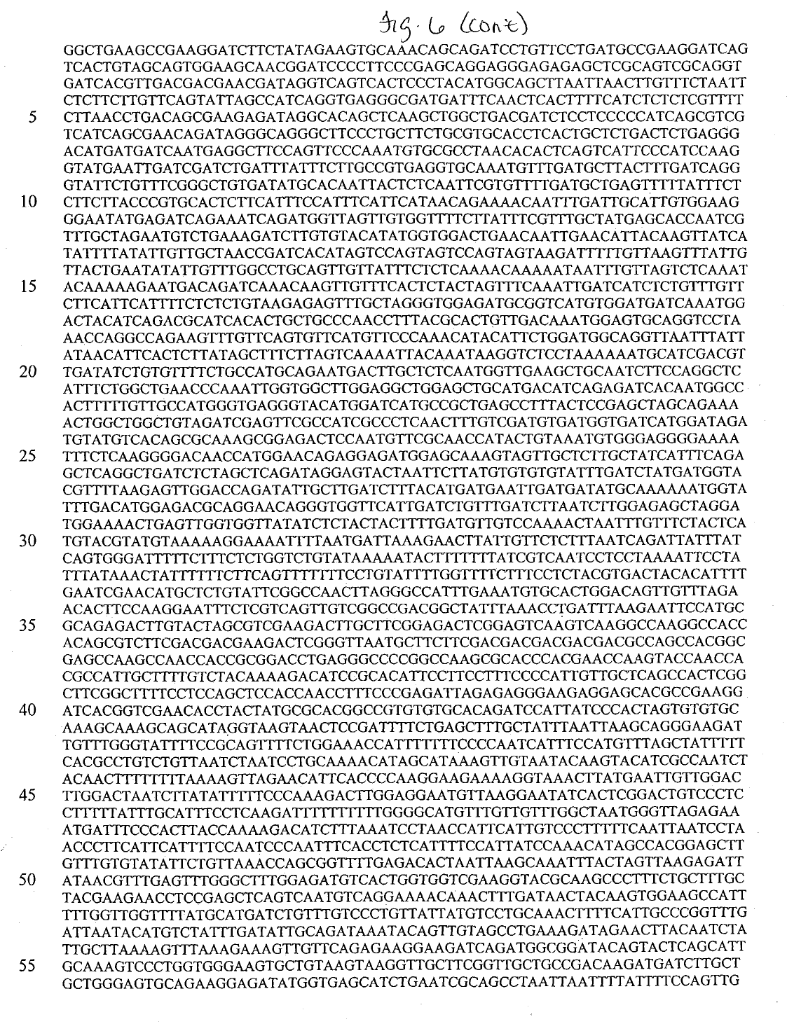

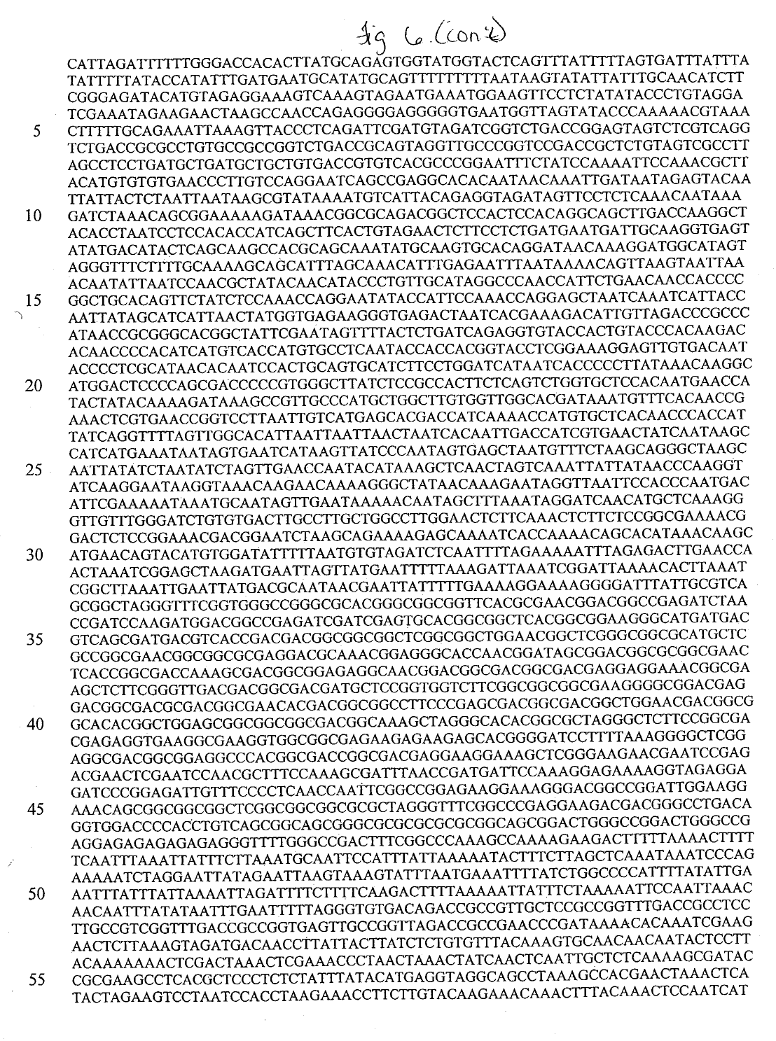

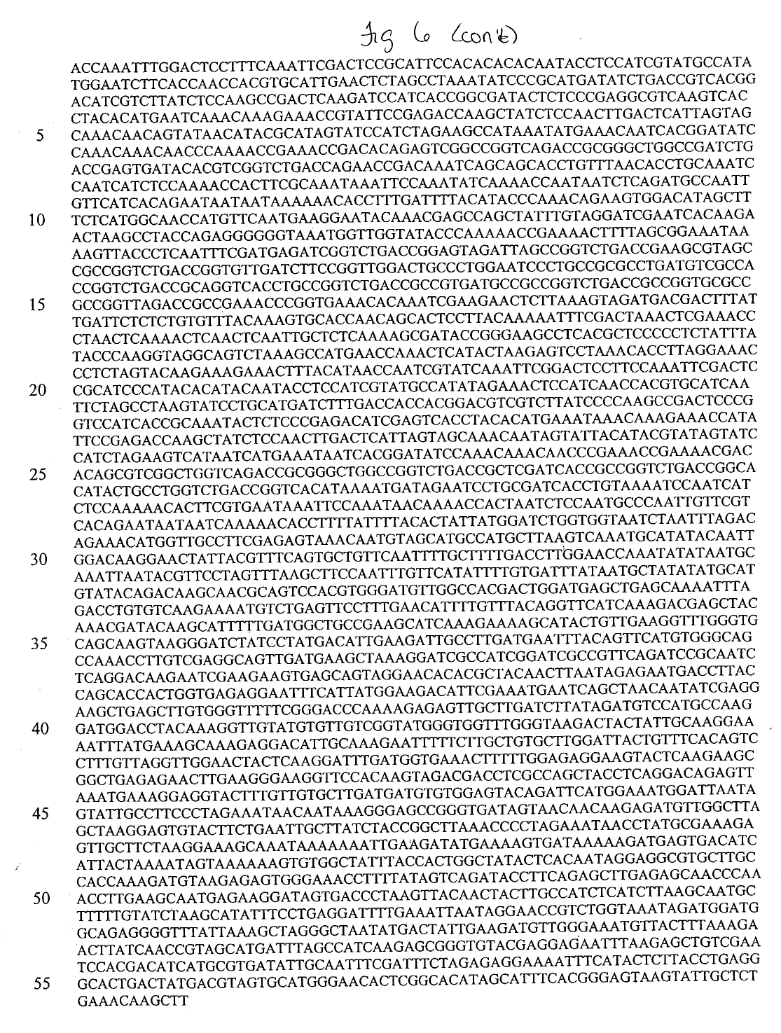

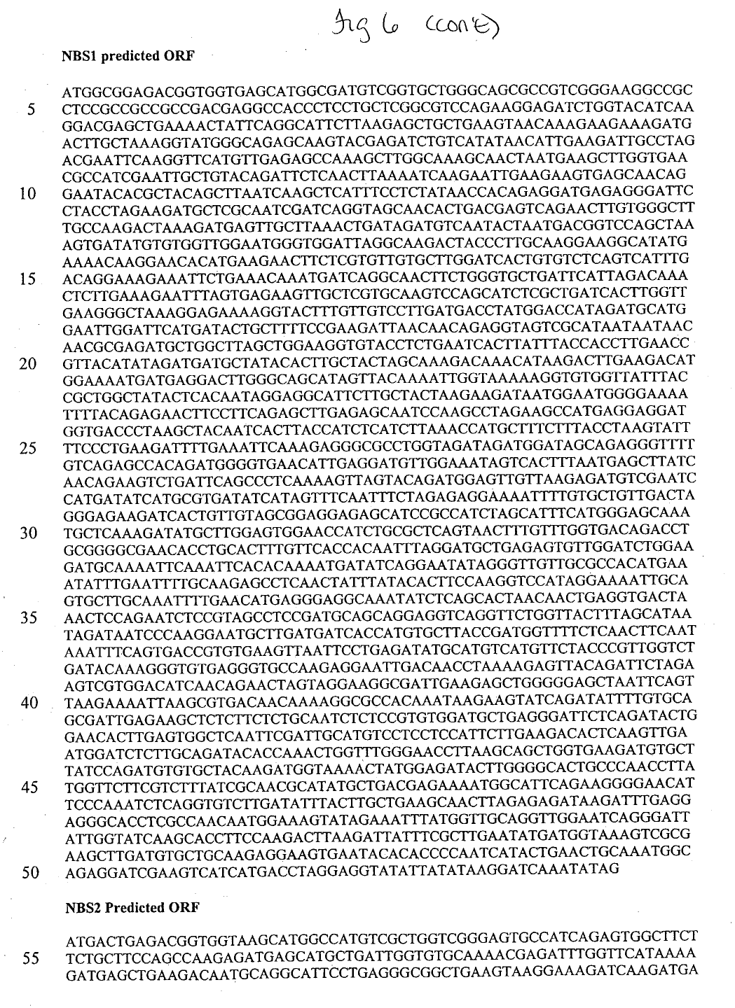

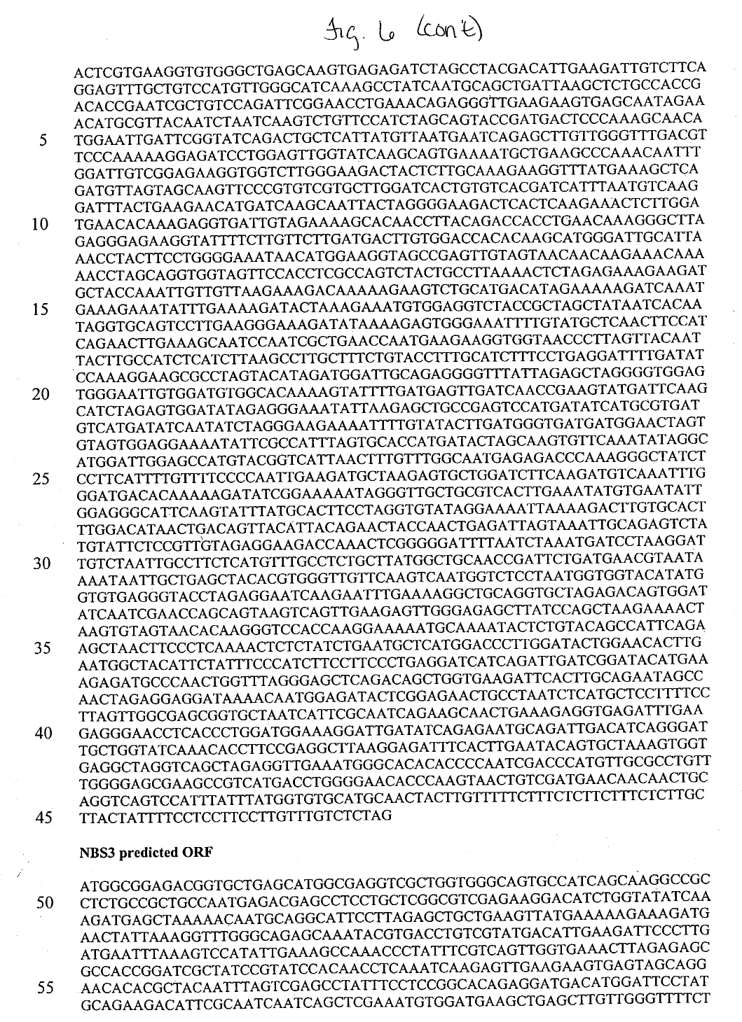

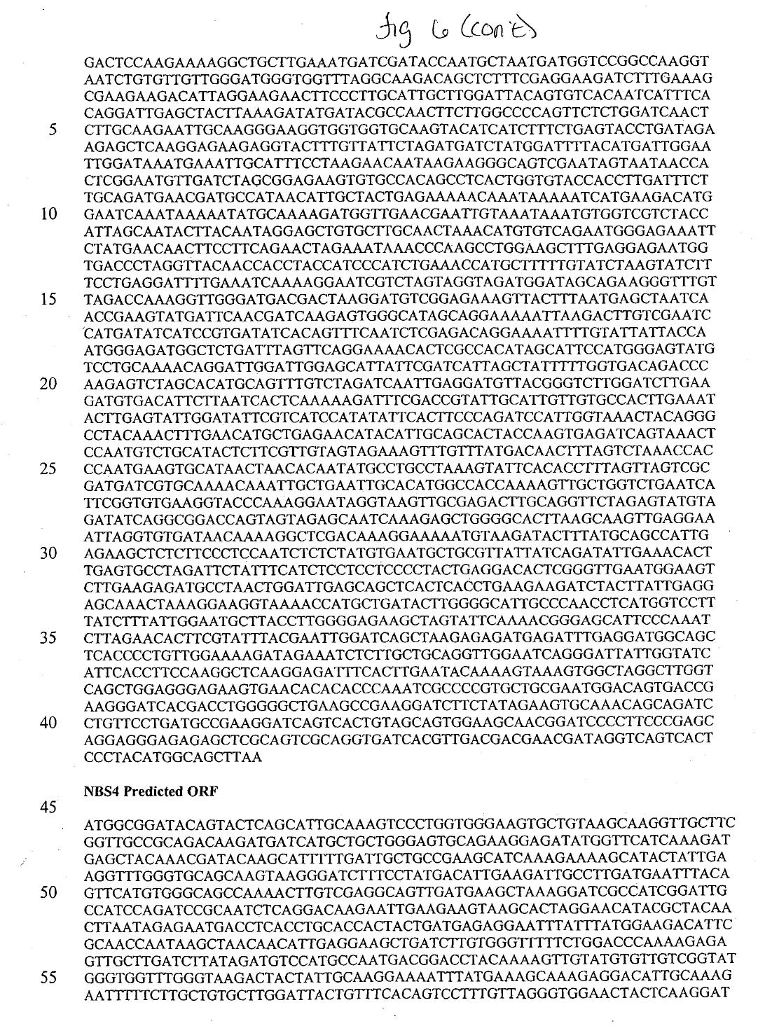

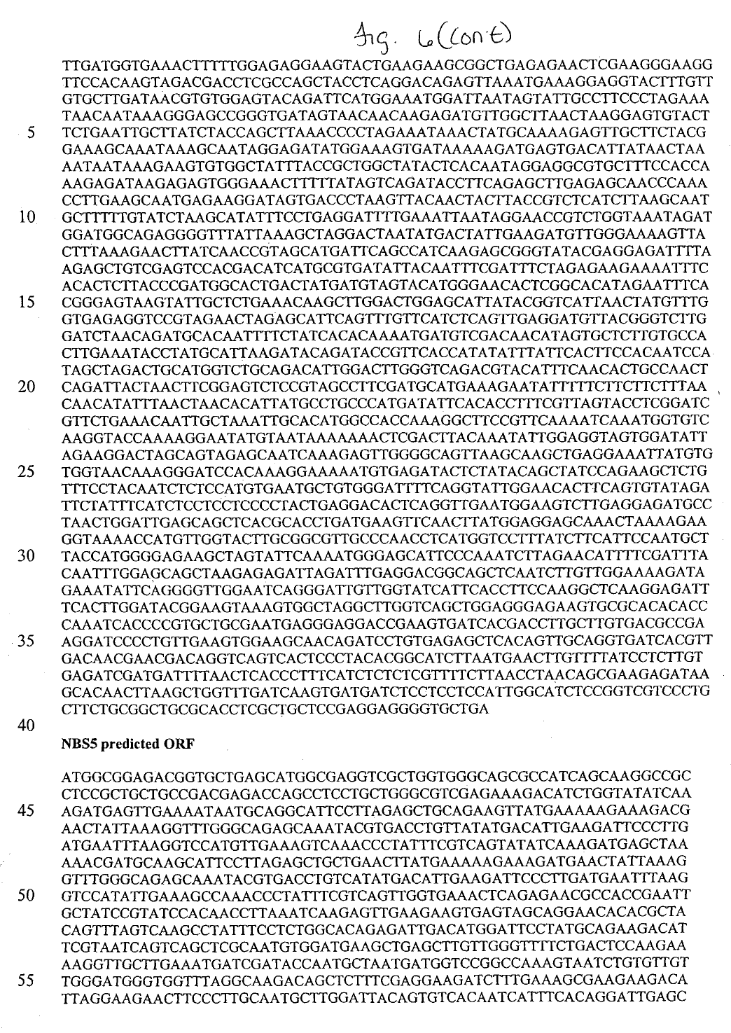

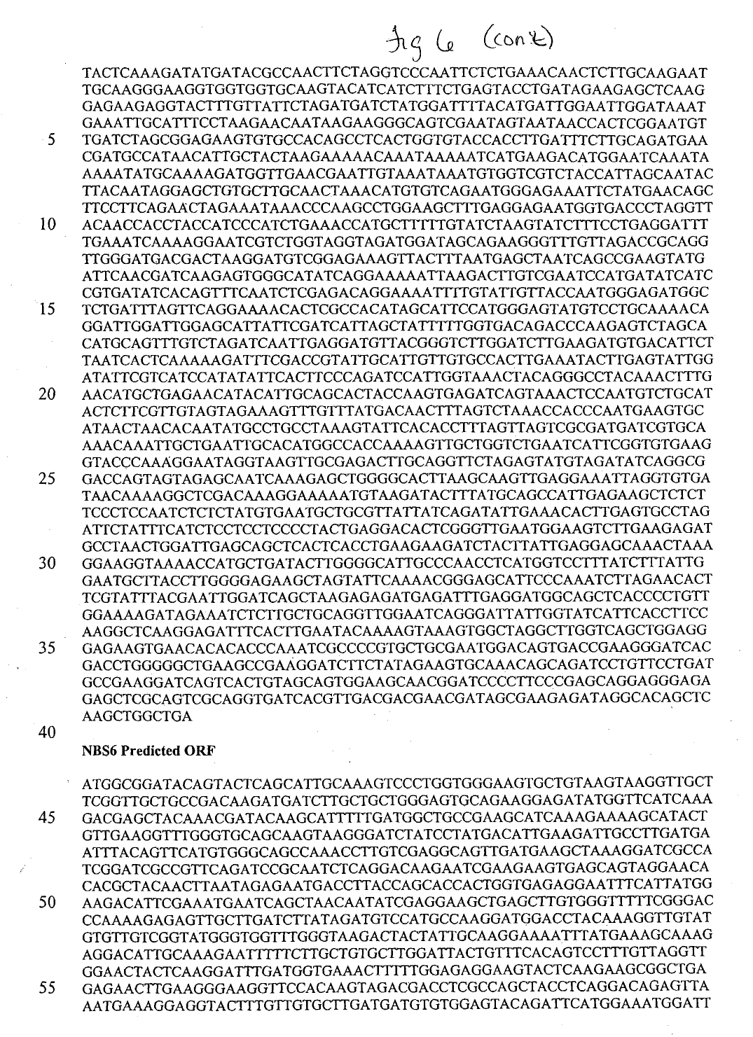

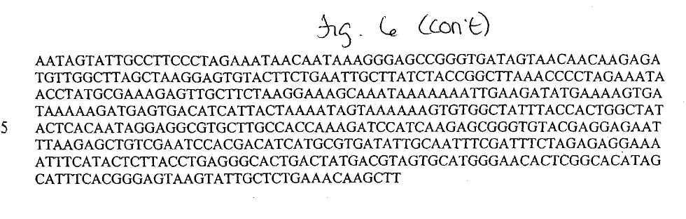

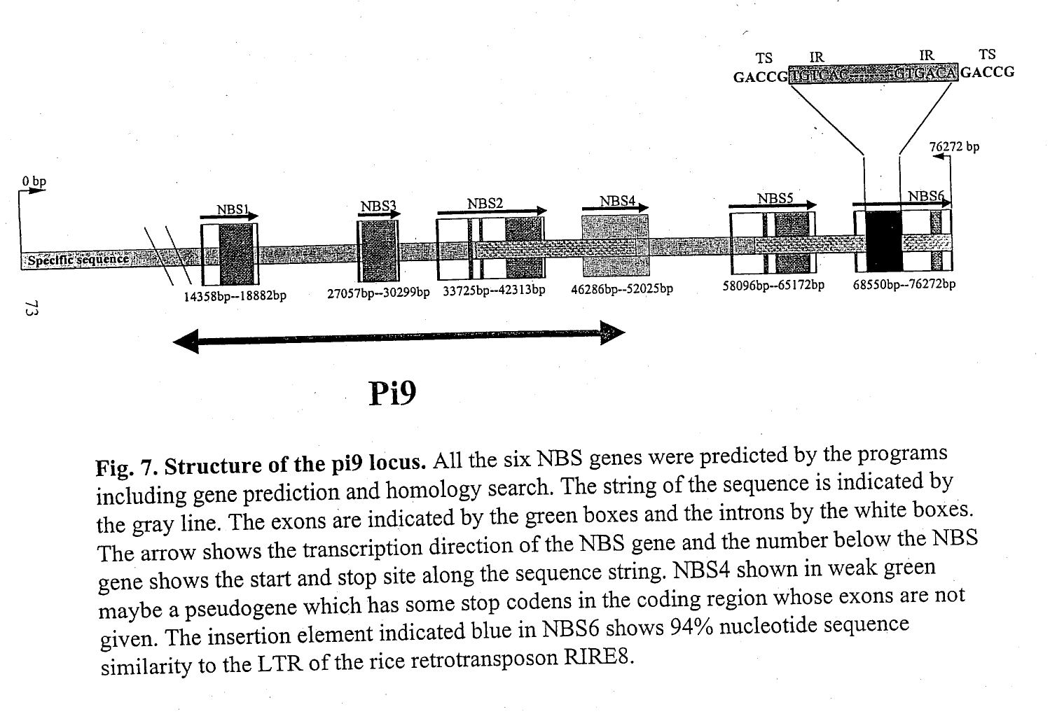

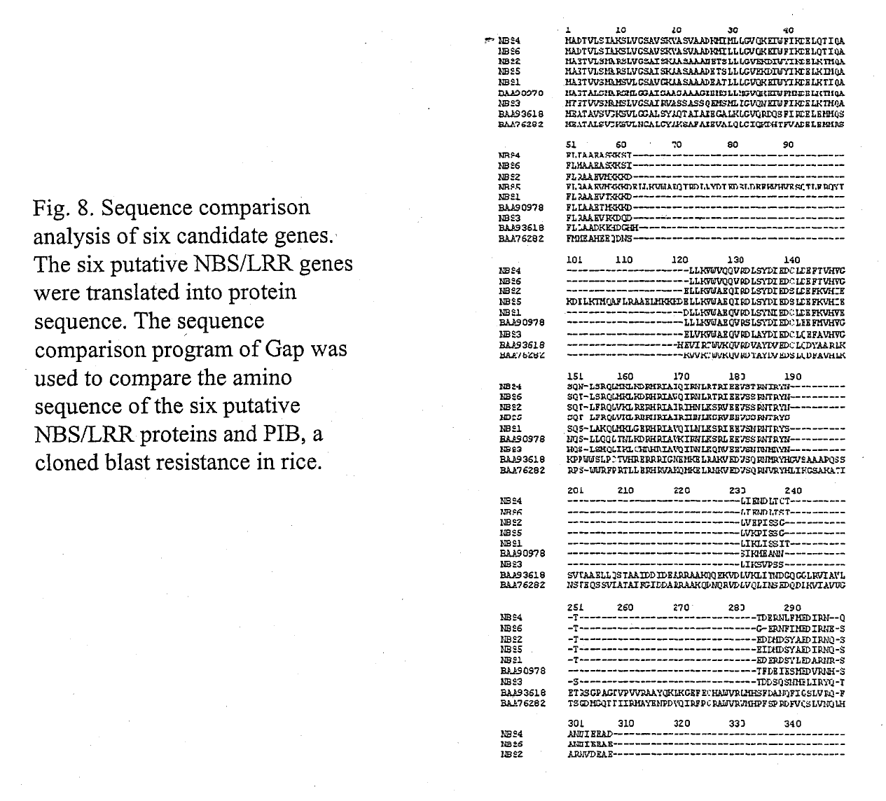

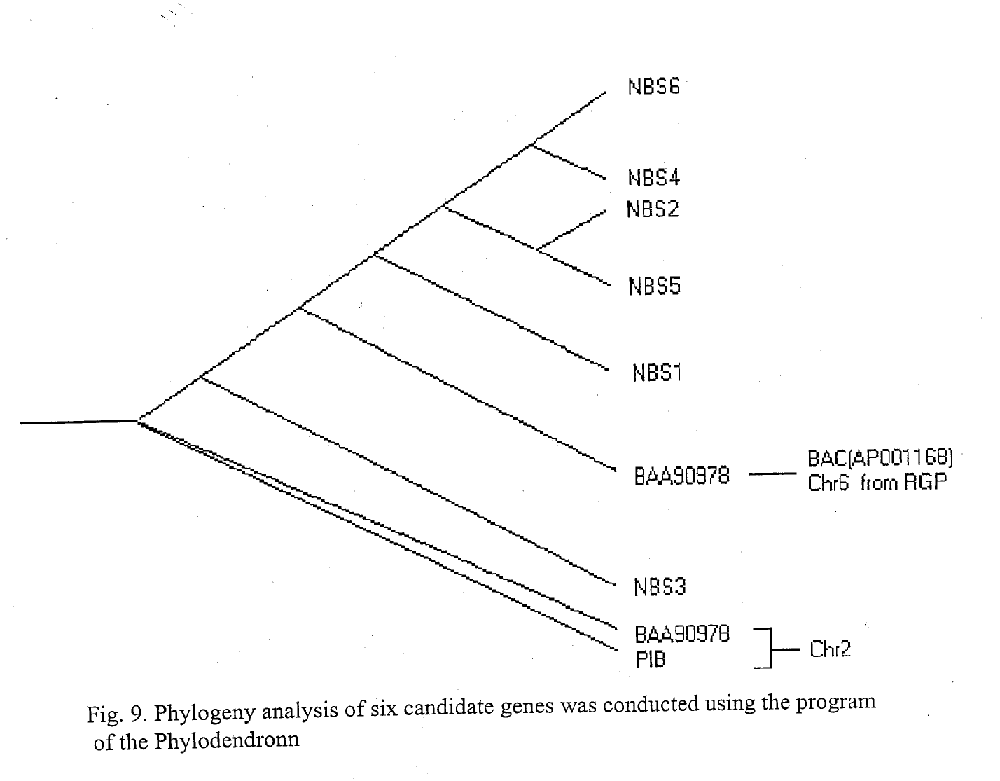

The six putative NBS/LRR genes were translated into protein sequence. The sequence comparison program of Gap was used to compare the amino sequence of the six putative NBS/LRR proteins and PIB, a cloned blast resistance in rice (Wang et al. 1999). The identity and similarity of all the NBS/LRR proteins were shown in Table 2 and the alignment of the candidate genes are shown in FIG. 8. The NBS2 and NBS5 shows 98% of identity in amino acid sequence each other and NBS4 and NBS6 shows 93%. All the six NBS/LRR protein found in Pi9 locus show higher than 28% identity in amino acid sequence to PIB protein. The multiple sequence alignment of the six NBS/LRR proteins was done by the program of Clustalw (accurate), which was shown in FIG. 7. A phylogeny analysis of six candidate genes was conducted using the program of the Phylodendronn (FIG. 9). The analysis revealed a similar result on the relationship of the six candidate genes with that obtained using the Gap program.

A Possible Duplication Event in the NBS/LRR Gene Cluster

Based on the sequence identity in the nucleotide sequence, two sequence fragments with high homology each other were identified (FIG. 6). The sequence from 38882 bp to 44118 bp shows 98% of identity to the sequence from 61740 bp to 66982, which are corresponding to the NBS2 and NBS5 separately. The sequence from 46029 bp to 49812 bp shows 94% of identity to the sequence from 68294 bp to 76251 bp which are corresponding to the NBS4 and NBS6 separately. The high identity of the genomic DNA region in Pi9 locus imply that one duplication event occurred during the evolution of this resistance gene locus. The retrotransposon inserted into the NBS6 gene may have occurred after the duplication event.

Fine-Mapping of the Pi9 Locus with NBS/LRR Genes

To pinpoint the Pi9 gene in the BAC contig, all the NBS/LRR candidate genes were used in Southern hybridization with a total of 596 plants (79 F₂, 55 F₃ and 462 F₅ plants). No recombination was observed between either NBS1, NBS2 or NBS3 with Pi9. Only one recombinant was found between NBS4 and Pi9 whereas two recombinants were found between NBS5 and NBS6 with Pi9. These results indicate that the Pi9 gene lies between BAC end 12R and candidate gene NBS4 (FIG. 7).

Screen for cDNAs Clone from the Pi9 cDNA Library

To isolate cDNA clones at the Pi9 locus, a cDNA library was made using the total RNA isolated from the infected leaf tissues of the Pi9 line 75-1-127. Leaf tissues were harvested at 12 and 24 hrs after inoculated with blast isolate PO₆-6. Equal amount of mRNAs from the two time points were mixed for the first strand cDNA synthesis. Detailed procedures were followed according to the manufacturers instruction (GIBCO-BRL, USA). About 7.2 million individual clones with average insert size of 1.5 kb were stored in 200 384-well plates.

Using both NBS1 and NBS2 as probes in colony hybridization, two cDNA clones were identified. These two clones were fully sequenced. BLAST search indicated that both genes had some homology with known NBS/LRR disease resistance genes. Sequence analysis revealed that one of the cDNAs (1.8 kb) matched with NBS3 and another cDNA (2.3 kb) matched with NBS5. Both clones are not full length based on the predicted ATG site in the genomic sequence.

Transformation of BAC 12 into Susceptible Cultivar TP309

When BAC12 was digested with restriction enzyme NotI, two fragments (45 kb and 13 kb, respectively) were released from the clone. The 45-kb fragment contains NBS1, NBS2 and NBS3 while the 13 kb fragment contains NBS4. These two NotI fragments are cloned into our newly constructed pTAC8 vector. Recombinant clones as TAC45 and TAC13 were transferred to Agrobacterium strain LBA4404. TP309 was transformed with these two constructs using the procedure established in our lab (Yin and Wang, 1999). Sixty and fifty independently transformed lines were generated from the transformation of TAC45 and TAC, respectively. About 3 TI plants from each line were transplanted in pots with soil and kept in greenhouse. Plants were selfed to produce T2 seeds.

Disease Evaluation of Transgenic Plants

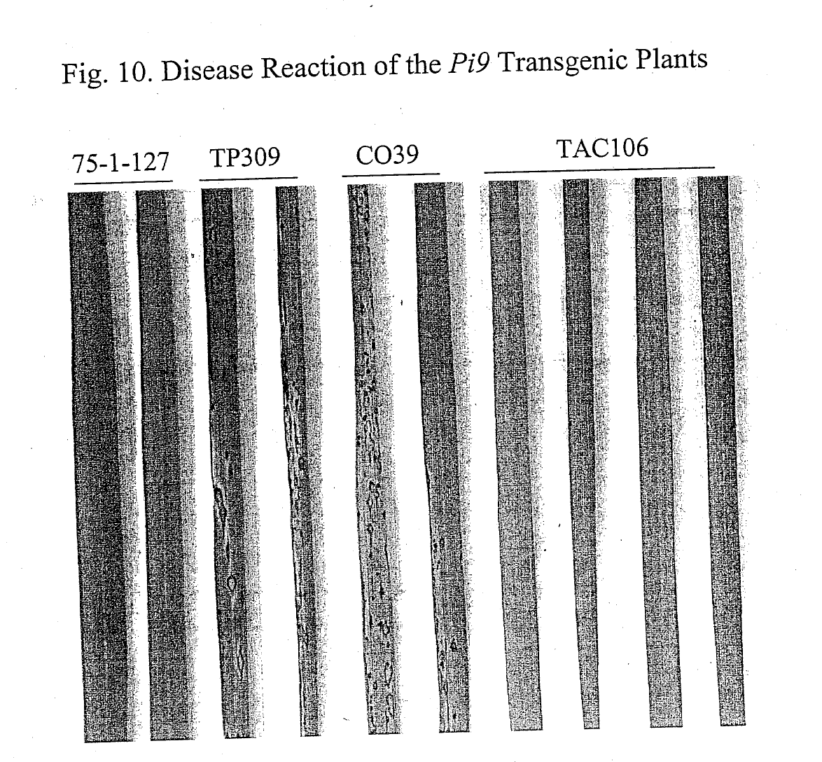

About 10-15 T2 seeds were sowed in trays and plants were growing in growth chamber. Eighteen days old plants (at 4 leaf stage) were inoculated with rice blast isolate PO6-6. Disease reaction was scored 7 days after inoculation based on a 0-5 scoring system. Inoculation results showed that all transgenic line transformed with TAC13 were highly susceptible to the isolate. In TAC45 transgenic lines, only one line (TAC106) showed segregation of resitance and susceptibility to blast. Among the 12 inoculated plants, 10 plants were resistance and 2 plant were susceptible (FIG. 10).

Small scale DNA was extracted from each plant. Primer pairs from NBS1, NBS2, NBS3 were used to check if these genes are present in the plants. The PCR result confirmed that NBS1, NBS2 and NBS3 were present in the resistant plants. Southern hybridization method will be used when enough leaf tissue for DNA extraction is available.

Mutant Generation from the Pi9 Plants

To identify mutants at the Pi9 locus or in the Pi9-mediated resistance pathway, about 20,000 75-1-127 seeds (carrying the Pi9 gene) was treated with the chemical mutagen DEB. Seeds were divided into two parts and treated with the chemical at concentrations of 0.04 and 0.06%, respectively. About 70% of the germination rate from 0.06% treatment and 80% of germination rate from 0.04% treatment were observed. Approximately, seed from 12,000 M1 plants were harvested.

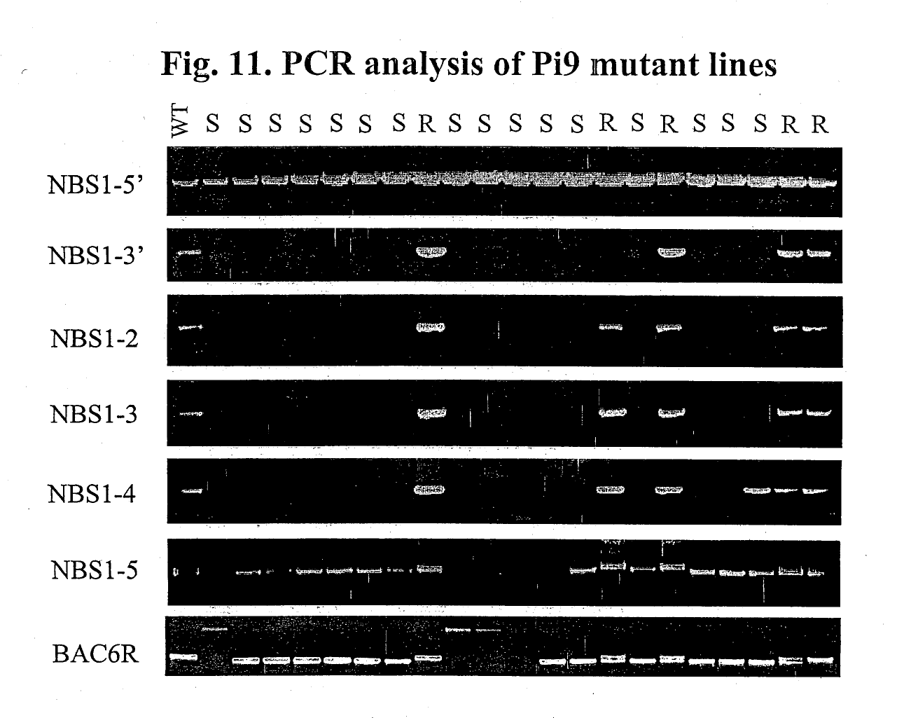

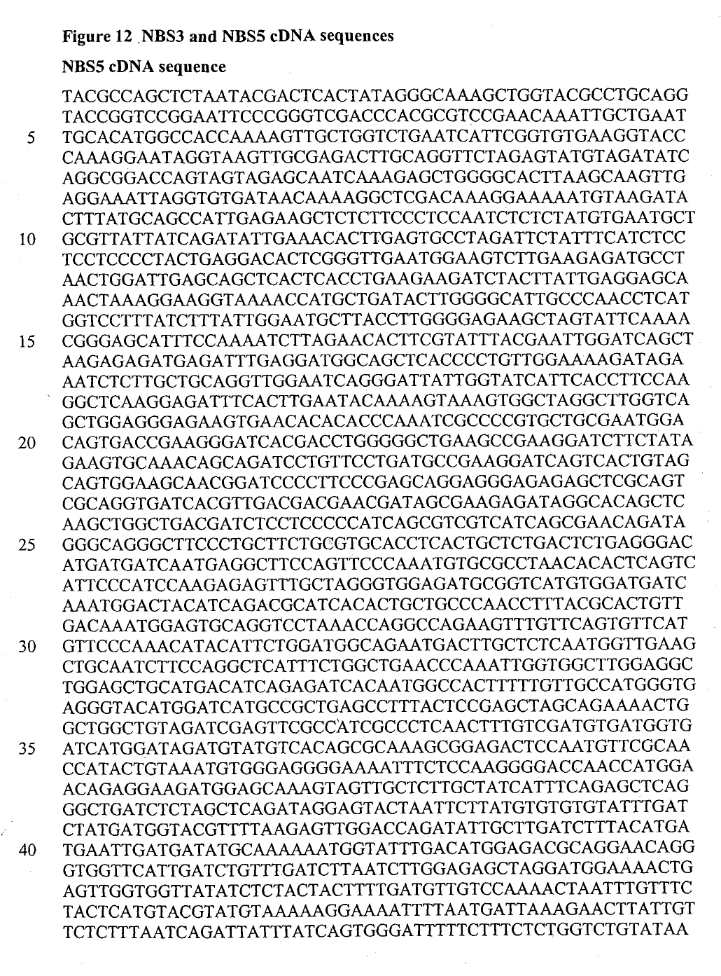

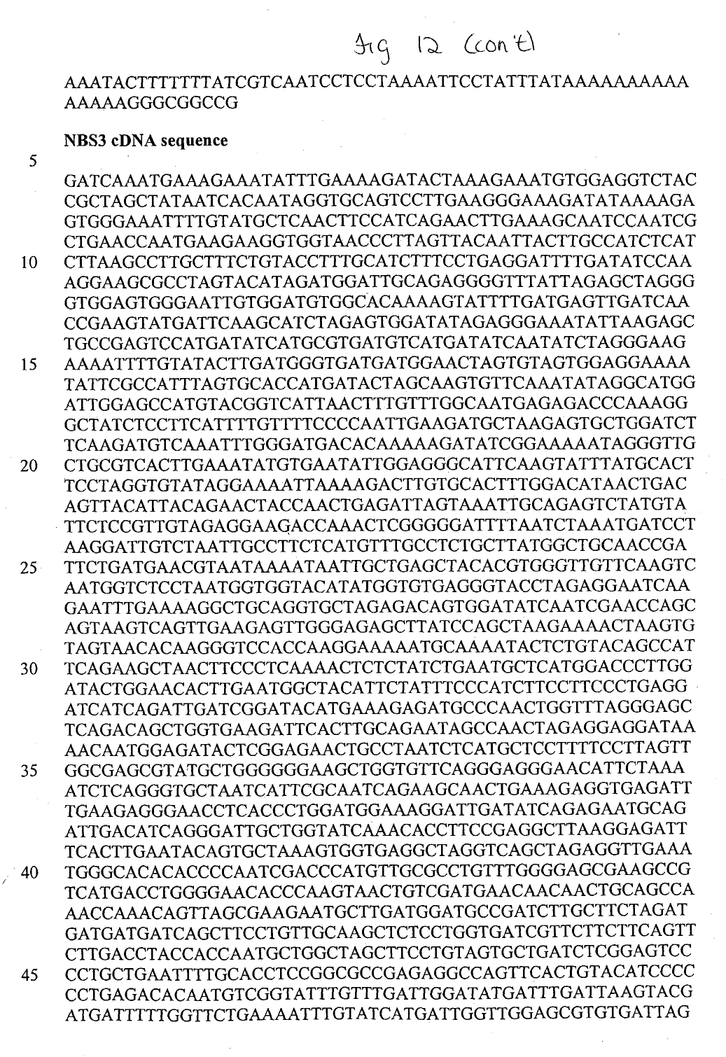

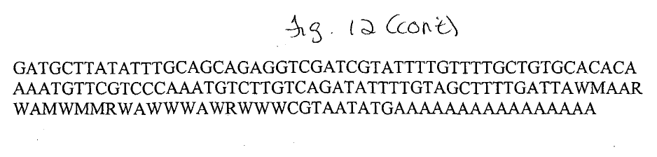

Bulk M2 seeds of the mutant population were sowed in a soil. Three weeks old plants were inoculated with PO6-6. Plants with visible lesions were picked 6 days after inoculation. Selfed seeds were harvested from the putative susceptible M2 plants. To confirm the disease reaction of the selected plants, inoculation was carried out in M3 generation. Lines showed typical susceptible lesions are transplanted and DNA was extracted from each plant for PCR and Southern analysis. PCR analysis with 5 NBS genes showed that NBS2 and NBS3 were deleted in all the susceptible mutant lines (FIG. 11).

### DEFINITIONS

By “cDNA” is meant DNA that is complementary to and derived from a mRNA.

By “complementarity” is meant a nucleic acid that can form hydrogen bond(s) with other nucleic acid sequences either through traditional Watson-Crick or other non-traditional types of base paired interactions.

By “constitutive promoter” is meant promoter elements that direct continuous gene expression in all cell types and at all times (i.e., actin, ubiquitin, CaMV ³⁵S, 35T, and the like).

By “developmental specific” promoter is meant promoter elements responsible for gene expression at specific plant developmental stages, such as in early or late embryogenesis and the like.

By “enhancer” is meant nucleotide sequence elements which can stimulate promoter activity such as those from maize streak virus (MSV) protein leader sequence, alfalfa mosaic virus protein leader sequence, alcohol dehydrogenase intron 1, and the like.

By “expression” as used herein, is meant the transcription and stable accumulation of mRNA inside a plant cell. Expression of genes also involves transcription of the gene to create mRNA and translation of the mRNA into precursor or mature proteins.

By “foreign” or “heterologous gene” is meant a gene encoding a-protein whose exact amino acid sequence is not normally found in the host cell, but is introduced by standard gene transfer techniques.

By “gene” is meant to include all genetic material involved in protein expression including chimeric DNA constructions, genes, plant genes and portions thereof, and the like.

By “genome” is meant genetic material contained in each cell of an organism and/or virus and the like.

By “inducible promoter” is meant promoter elements which are responsible for expression of genes in response to a specific signal such as: physical stimuli (heat shock genes); light (RUBP carboxylase); hormone (Em); metabolites, chemicals, stress and the like.

By “plant” is meant a photosynthetic organism including both eukaryotes and prokaryotes.

By “promoter regulatory element” is meant nucleotide sequence elements within a nucleic fragment or gene which controls the expression of that nucleic acid fragment or gene. Promoter sequences provide the recognition for RNA polymerase and other transcriptional factors required for efficient transcription. Promoter regulatory elements from a variety of sources can be used efficiently in plant cells to express gene constructs. Promoter regulatory elements are also meant to include constitutive, tissue-specific, developmental-specific, inducible promoters and the like. Promoter regulatory elements may also include certain enhancer sequence elements and the like that improve transcriptional efficiency.

By “tissue-specific” promoter is meant promoter elements responsible for gene expression in specific cell or tissue types, such as the leaves or seeds (i.e., zein, oleosin, napin, ACP, globulin and the like).

By “transformation” is meant a process of introducing an exogenous DNA sequence (e g., a vector, a recombinant DNA molecule) into a cell or protoplast in which that exogenous DNA is incorporated into a chromosome or is capable of autonomous replication.

By “transformed cell” is meant a cell whose DNA has been altered by the introduction of an exogenous DNA molecule into that cell.

By “transgene” is meant an exogenous gene which when introduced into the genome of a host cell through a process such as transformation, electroporation, particle bombardment, and the like, is expressed by the host cell and integrated into the cells genome such that the trait or traits produced by the expression of the tansgene is inherited by the progeny of the transformed cell.

By “transgenic cell” is meant any cell derived or regenerated from a transformed cell or derived from a transgenic cell. Exemplary transgenic cells include plant calli derived from a transformed plant cell and particular cells such as leaf, root, stem, e.g., somatic cells, or reproductive (germ) cells obtained from a transgenic plant.

By “transgenic plant” is meant a plant or progeny thereof derived from a transformed plant cell or protoplast, wherein the plant DNA contains an introduced exogenous DNA molecule not originally present in a native, non-trarsgenic plant of the same strain. The tenms “transgenic plant” and “transformed plant” have sometimes been used in the art as synonymous terms to define a plant whose DNA contains an exogenous DNA molecule. However, it is thought more scientifically correct to refer to a regenerated plant or callus obtained from a transformed plant cell or protoplast as being a transgenic plant, and that usage will be followed herein.

By “vector” is meant a DNA molecule capable of replication in a host cell and/or to which another DNA segment can be operatively linked so as to bring about replication of the attached segment. A plasmid is an exemplary vector.

Nucleic Acid Molecules

Nucleic acid molecules of the present invention include, without limitation, nucleic acid molecules having a nucleic acid sequence selected from the group consisting of SEQ ID NOs: 1-84, 86, 88, 90, 92, 94, 96, and 97 and complements thereof. A subset of the nucleic acid molecules of the present invention includes nucleic acid molecules that encode the NBS1, NBS2, NBS3, NBS4, NBS5, or NBS6 protein or a variant thereof. Such variants comprise an amino acid sequence which is at least 90% identical to SEQ ID NO. 85, SEQ ID NO. 87, SEQ ID NO. 89, SEQ ID NO. 91, SEQ ID NO. 93, or SEQ ID NO. 95. The amino acids which are not identical, preferably, are conservative amino acid substitutions.

Another subset of the nucleic acid molecules of the present invention includes nucleic acid molecules that can be used as probes or primers for selecting or identifying plants whose genome comprises an NBS rice blast resistant allele. A list of such primers is attached to this application. The listed primers were designed based on the genomic sequences from BAC clones, DNA markers and other genomic clones. These primers can be used in gene amplification and marker-aided selection.

Fragment nucleic acid molecules may comprise significant portion(s) of, or indeed most of, these nucleic acid molecules. In preferred embodiments, the fragments may comprise smaller polynucleotides, e.g., oligonucleotides having from about 20 to about 250 nucleotide residues and more preferably, about 40 to about 100 nucleotide residues. Such fragments are useful as probes for identifying plants whose genome includes an NBS rice blast resistant allele. In another preferred embodiment, fragment molecules may be at least 15 nucleotides and are useful as primers for identifying or selecting plants whose genome includes an NBS rice resistant allele.

The nucleic acids may be single-stranded or double stranded. Such nucleic acids may be DNA or RNA molecules

The term “isolated,” as used herein, refers to a molecule separated from substantially all other molecules normally associated with it in its native state. More preferably an isolated molecule is the predominant species present in a preparation. An isolated molecule may be greater than 60% free, preferably 75% free, more preferably 90% free, and most preferably 95% free from the other molecules (exclusive of solvent) present in the natural mixture. The term “isolated” is not intended to encompass molecules present in their native state.

It is understood that the nucleic acids of the present invention, particularly the probes and primers, may be labeled with reagents that facilitate detection of the agent, e.g., fluorescent labels, (Prober et al., Science 238:336-340 (1987); Albarella et al., EP 144914), chemical labels, (Sheldon et al., U.S. Pat. No. 4,582,789; Albarella et al., U.S. Pat. No. 4,563,417), and modified bases, (Miyoshi et al., EP 119448) including nucleotides with radioactive elements, e.g., .sup.32P, .sup.33P, .sup.35S or .sup.1251, such as .sup.32P dCTP.

It is further understood, that the present invention provides recombinant bacterial, animal, fungal and plant cells, plasmid and viral constructs comprising the isolated nucleic acids of the present invention.

Nucleic acid molecules or fragments thereof of the present invention are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit “complete complementarity,” i.e., each nucleotide in one sequence is complementary to its base pairing partner nucleotide in another sequence. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Nucleic acid molecules which hybridize to other nucleic acid molecules, e.g., at least under low stringency conditions are said to be “hybridizable cognates” of the other nucleic acid molecules. Conventional stringency conditions are described by Sambrook et al., Molecular Cloning, A Laboratory Manual, 2nd Ed., Cold Spring Harbor Press, Cold Spring Harbor, N.Y. (1989) and by Haymes et al., Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, 6.0.times. sodium chloride/sodium citrate (SSC) at about 45.degree. C., followed by a wash of 2.0.times. SSC at 50.degree. C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0.times. SSC at 50.degree. C. to a high stringency of about 0.2.times. SSC at 50.degree. C. In addition, the temperature in the wash step can be increased from low stringency conditions at room temperature, about 22.degree. C., to high stringency conditions at about 65.degree. C. Both temperature and salt may be varied, or either the temperature or the salt concentration may be held constant while the other variable is changed.

In a preferred embodiment, a nucleic acid of the present invention will specifically hybridize to at least 15 contiguous nucleotides in one or more of the nucleic acid molecules set forth in SEQ ID NO: 83, 85, 87, 89, 91, 93, or 95 or complements thereof under moderately stringent conditions, for example at about 2.0.times. SSC and about 65.° C.

In a particularly preferred embodiment, a nucleic acid of the present invention will include those nucleic acid molecules that specifically hybridize to at least 15 contiguous nucleotides in one or more of the nucleic acid molecules set forth in SEQ ID NO: 85, 87, 89, 91, 93, or 95 or complements thereof under high stringency conditions such as 0.2.times. SSC and about 65° C.

In one aspect of the present invention, the nucleic acid molecules of the present invention comprise one or more of the nucleic acid sequences set forth in SEQ ID NOs. 84, 86, 88, 90, 92, or 94 or complements thereof or fragments of either. In another aspect of the present invention, one or more of the nucleic acid molecules of the present invention share at least 90% sequence identity with one or more of the nucleic acid sequences set forth in SEQ ID NO: 84, 86, 88, 90, 92, or 94 or complements thereof.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. “Percent identity” is the identity fraction times 100.

Useful methods for determining sequence identity are disclosed in Guide to Huge Computers, Martin J. Bishop, ed., Academic Press, San Diego, 1994, and Carillo, H., and Lipton, D., SIAM J Applied Math (1988) 48:1073. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990); version 2.0 or higher of BLAST programs allows the introduction of gaps (deletions and insertions) into alignments; BLASTX can be used to determine sequence identity between a polynucleotide sequence query and a protein sequence database; and, BLASTN can be used to determine sequence identity between between sequences.

For purposes of this invention “percent identity” shall be determined using BLASTX version 2.0.14 (default parameters), BLASTN version 2.0.14, or BLASTP 2.0.14.

The isolated nucleic acid molecules that encode SEQ ID NOs 85, 87, 89, 91, 93, and 95 can be used to produce NBS 1, NBS 2, NBS 3, NBS4, NBS 5, and NBS6 proteins using any of a variety of methods known to those skilled in the art. The amino acid sequence of the NBS1 protein is shown in FIG. 8 and set forth in SEQ ID NO. 85. One embodiment of a nucleotide sequence encoding the NBS1 protein is shown in Fig. and set forth in SEQ ID NO. 84. The amino acid sequence of the NBS2 protein is shown in FIG. 8 and set forth in SEQ ID NO. 87. One embodiment of a nucleotide sequence encoding the NBS2 protein is shown in FIG. 8 and set forth in SEQ ID NO. 86. The amino acid sequence of the NBS3 protein is shown in FIG. 8 and set forth in SEQ ID NO 89. One embodiment of a nucleotide sequence encoding the NBS3 protein is shown in Fig. and set forth in SEQ ID NO. 88. The amino acid sequence of the NBS4 protein is shown in FIG. 8 and set forth in SEQ ID NO 91. One embodiment of a nucleotide sequence encoding the NBS4 protein is shown in FIG. 8 and set forth in SEQ ID NO. 90. The amino acid sequence of the NBS6 protein is shown in FIG. 8 and set forth in SEQ ID NO 95. One embodiment of a nucleotide sequence encoding the NBS5 protein is shown in FIG. 8 and set forth in SEQ ID NO. 94. The proteins may be used for various purposes. One purpose for the proteins is as antigens to cause production of antibodies that react with the proteins. The present invention encompasses such antibodies.

Modification and changes may be made in the structure of the peptides of the present invention and DNA segments which encode them and still obtain a functional molecule that encodes a protein or peptide with desirable characteristics. The following is a discussion based upon changing the amino acids of a protein to create an equivalent, or even an improved, second-generation molecule. In particular embodiments of the invention, mutated proteins are contemplated to be useful for increasing the rice blast disease resistance activity of the protein, and consequently increasing the activity and/or expression of the recombinant transgene in a plant cell. The amino acid changes may be achieved by changing the codons of the DNA sequence, according to the codons given in Table 3.

TABLE 3

| Amino Acid | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (p.4); threonine (−7); serine (−8); trtyptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, ie., still obtain a biological functionally equivalent protein. In making such changes, the substitution of amino acids whose hydropathic indices are within .+−0.2 is preferred, those which are within .+−.1 are particularly preferred, and those within .+−.0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As described in U.S. Pat. No. 4,554,101, the following hydrophilicity values have been assigred to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (O); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent, and in particular, an immunologically equivalent protein. In such changes, the substitution of amino acids whose hydrophilicity values are within .+−0.2 is preferred, those which are within .+−0.1 are particularly preferred, and those within .+−.0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take variots of the foregoing characteristics into consideration are well known to those of skill in th. art and include: arginine and lysine; glutamate and aspartate; serine and threonine; glutamine and asparagine; and valine, leucine and isoleucine.

Site-specific mutagenesis is a technique useful in the preparation of individual peptides, or biologically functional equivalent proteins or peptides, through specific mutageneis of the underlying DNA. The technique further provides a ready ability to prepare aid test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed.

In general, the technique of site-specific mutagenesis is well known in the art, as exemplified by various publications. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art. Double stranded plasmids are also routinely employed in site directed mutagenesis which eliminates the step of transferring the gene of interest from a plasmid to a phage.

The preparation of sequence variants of the selected peptide-encoding DNA segments using site-directed mutagenesis is provided as a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants.

Concerning the amino acid sequences of the inventive proteins disclosed herein, variants of those proteins are also encompassed within the scope of this invention. Such encompassed variants have at least 90% amino acid sequence identity with one or more of the inventive proteins disclosed herein. Such variants include, for instance, proteins wherein one or more amino acid residues are added or deleted at the N- or C-terminus of the sequence of the disclosed protein sequences or one or more amino acid residues within the disclosed protein sequences are substituted, preferably with a conservative amino acid. Ordinarily, the disclosed protein sequence variants will have at least about 90% amino acid sequence identity, more preferably at least about 95% amino acid sequence identity, with the amino acid sequence of the disclosed proteins. Percent (%) amino acid sequence identity with respect to the sequence herein is defined as the percentage of amino acid residues in a candidate sequence that are identical with the amino acid residues in the disclosed protein sequences, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN.TM. or Megalign (DNASTAR) software. Those skilled in the art can determine appropriate parameters for measuring alignment, including any algorithms needed to achieve maximal alignment over the full length of the sequences being compared.

Preferably, the deletions and additions are located at the amino terminus, the carboxy terminus, or both, of the disclosed protein sequences. Amino acid substitutions are generally based on the relative similarity of the amino acid side-chain substituents, for example, their hydrophobicity, hydrophilicity, charge, size, and the like.

It is known in the art that certain amino acids may be substituted by other amino acids having a similar hydropathic index score and a similar hydrophilicity value and still result in a protein with similar biological activity, i.e., still obtain a biological functionally equivalent protein. It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

Transgenic Plants Comprising an NBS Rice Blast Resistant Allele

The present invention provides a method of producing a transgenic plant whose genome comprises an NBS rice resistant allele. The method comprises introducing a transgene or DNA construct comprising a nucleic acid that encodes an NBS 1 protein, an NBS2 protein, an NBS3 protein, an NBS4 protein, an NBS5 protein, and NBS6 protein or combinations thereof and a promoter which is operably linked to said nucleic acid into a plant cell or protoplast, and regenerating a plant from said plant cell or said protoplast.

Another aspect of the invention comprises a transgenic plant whose genome comprises a transgene or DNA expression construct that encodes and expresses the present NBSI, NBS2, NBS32, NBS4, NBS5, or NBS6 protein.

Types of Plants in Which the Invention can be Used

The transgene of the present invention can be introduced in a variety of non-transgenic host plants. The grasses are one family of plants that can be used to produce the present transgenic plants, however, the invention may be used in other families of plants. The grass family comprises the plants in the family Gramineae (also called Poaceae). This family comprises plants such as maize, wheat, rice, barley, turfgrass, ryegrass, stall fascue grass, other turf plants, orghum, rye, and sugar cane. The plants comprising rice is preferably used to practice the invention. One genus in the rice family is Oryza. Within the genus Oryza, a variety of species are found. These species comprise Octopus vulgaris, Onchocerca cervicalis, Onchocerca volvulus, Oryctolagus cuniculus, Oryza australiensis, Oryza brachyantha, Oryza latifolia, Oryza minuta, Oryza nivarra, Oryza officinalis, Oryza punctata, Oryza rufipogon, Oryza sativa, Oryza longistaminata, Oryza glaberrima, Oryza eichingeri, Oryza grandiglumis, Oryza perennis, Oryza glumaepatula, Oryza meridionalis, Oryza alta and other genera within this species. All of these species may be used in practice of the present invention.

Types of Diseases and Infective Organisms the Invention Can be Used to Prevent

The present invention, especially the genes of the present invention, are used to make strains of plants that are resistance to particular diseases, the diseases being caused by infective organisms. The genes and proteins of the present invention can be used to prevent infection of any infective organisms to which the inventive genes and proteins provide protection. Preferably, the present invention is used to prevent, lessen the severity of, or lessen the occurrence of rice blast disease. Rice blast disease is caused by organisms of the genus Magnaporthe (also called Pvricularia). One such organism is Magnaporthe grisea. However, the present invention is not limited to protection against these particular infective organisms. The inventive genes and proteins may provide protection against infection (i.e., resistance to infection) against other infective organisms.

Aside from rice, the Magnaporthe grisea fungus can also attack more than fifty other species of grasses and sedges. The present invention can be practiced using any of the species of plants that the organisms causing rice blast disease can infect.

The effects of rice blast disease on plants and the identification of the disease in plants are well known in the art and are described in various publications such as MP 645, Rice Blast: Identification and Control and MP 646, Rice Sheath Blight Control. These publications are incorporated herein by reference and are available from the Delta Center, Missouri Agricultural Experiment Station, P.O. Box 160, Portageville, Mo. 63873, or are be available a on the World Wide Web at (aes.missouri.edu/delta).

Preparation of the Transgenic Plant

The genes of the present invention are introduced into and expressed in the plants that are susceptible to Magnaporthe grisea. One or more of the genes, or all of the genes can be introduced into plants to provide the desired result of resistance to the rice blast disease fungus. There are a variety of methods by which the genes can be introduced into plants and transferred between plants. When rice plants are used, genes can be introduced into the rice using various methods of transformation. Such plants that contain an introduced gene are referred to as transgenic plants. Such transformation methods include biolistic methods, Agrobacterium tumefaciens-based methods, and methods involving direct gene transfer into protoplasts. Many of these methods are either described or referenced in a paper that is attached to and is part of this application. This paper is authored by Yin and Wang and was published in Theor Appl Genet in 2000. This paper is incorporated into the present application by reference. An article authored by V. L. Muniz de Padua et. al. published in Plant Molecular Biology Reporter in 2001, describes additional methods for transforming plant cells.

Once genes have been introduced into rice plants, the genes can be moved from plant to plant using various genetic methods, traditional breeding methods, that are well known to those skilled in the art. Such methods include crosses or genetic crosses between the plants. Techniques such as embryo rescue can also be used.

Methods for DNA transformation of plant cells include Agrobacterium-mediated plant transformation, protoplast transformation, gene transfer into pollen, injection into reproductive organs, injection into immature embryos and particle bombardment. Each of these methods has distinct advantages and disadvantages. Thus, one particular method of introducing genes into a particular plant strain may not necessarily be the most effective for another plant strain, but it is well known which methods are useful for a particular plant strain.

There are many methods for introducing transforming DNA segments into cells, but not all are suitable for delivering DNA to plant cells. Suitable methods are believed to include virtually any method by which DNA can be introduced into a cell, such as infection by A. tamefaciens and related Agrobacterium, direct delivery of DNA such as, for example, by PEG-mediated transformation of protoplasts (Ominilleh et al., 1993), by desiccation/inhibition-mediated DNA uptake, by electroporation, by agitation with silicon carbide fibers, by acceleration of DNA coated particles, etc. In certain embodiments, acceleration methods are preferred and include, for example, microprojectile bombardment and the like.

Technology for introduction of DNA into cells is well-known to those of skill in the art. Four general methods for delivering a gene into cells have been described: (1) chemical methods (Graham and van der Eb, 1973); (2) physical methods such as microinjection (Capecchi, 1980), electroporation (Wong and Neumann, 1982; Fromm et al., 1985) and the gene gun (Johnston and Tang, 1994; Fynan et al., 1993); (3) viral vectors (Clapp, 1993; Lu et al., 1993; Eglitis and Anderson, 1988a; 1988b); and (4) receptor-mediated mechanisms (Curiel et al., 1991; 1992; Wagner et al., 1992).

Electroporation

The application of brief, high-voltage electric pulses to a variety of animal and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of clones genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

The introduction of DNA by means of electroporation, is well-known to those of skill in the art. In this method, certain cell wall-degrading enzymes, such as pectin-degrading enzymes, are employed to render the target recipient cells more susceptible to transformation by electroporation than untreated cells. Alternatively, recipient cells are made more susceptible to transformation, by mechanical wounding. To effect transformation by electroporation one may employ either friable tissues such as a suspension culture of cells, or embryogenic callus, or alternatively, one may transform immature embryos or other organized tissues directly. One would partially degrade the cell walls of the chosen cells by exposing them to pectin-degrading enzymes (pectolyases) or mechanically wounding in a controlled manner. Such cells would then be recipient to DNA transfer by electroporation, which may be carried out at this stage, and transformed cells then identified by a suitable selection or screening protocol dependent on the nature of the newly incorporated DNA.

Microprojectile Bombardment

A further advantageous method for delivering transforming DNA segments to plant cells is microprojectile bombardment. In this method, particles may be coated with nucleic acids and delivered into cells by a propelling force. Exemplary particles include those comprised of tungsten, gold, platinum, and the like.

An advantage of microprojectile bombardment, in addition to it being an effective means of reproducibly stably transforming monocots, is that neither the isolation of protoplasts (Cristou et al., 1988) nor the susceptibility to Agrobacterium infection is required. An illustrative embodiment of a method for delivering DNA into maize cells by acceleration is a Biolistics Particle Delivery System, which can be used to propel particles coated with DNA or cells through a screen, such as a stainless steel or Nytex screen, onto a filter surface covered with corn cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing damage inflicted on the recipient cells by projectiles that are too large.

For the bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. Through the use of techniques set forth herein one may obtain up to 1000 or more foci of cells transiently expressing a marker gene. The number of cells in a focus which express the exogenous gene product 48 hours post-bombardment often range from 1 to 10 and average 1 to 3.

In bombardment transformation, one may optimize the prebombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of immature embryos.

Accordingly, it is contemplated that one may wish to adjust various of the bombardment parameters in small scale studies to fully optimize the conditions. One may particularly wish to adjust physical parameters such as gap distance, flight distance, tissue distance, and helium pressure. One may also minimize the trauma reduction factors (TRFs) by moditying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

The methods of particle-mediated transformation is well-known to those of skill in the art. U.S. Pat. No. 5,015,580 (specifically incorporated herein by reference) describes the transformation of soybeans using such a technique.

Agrobacterium-Mediated Transfer

Agrobacterium-mediated transfer is a widely applicable system for introducing genes into plant cells because the DNA can be introduced into whole plant tissues, thereb bypassing the need for regeneration of an intact plant from a protoplast. The use of Agrobactenum-mediated plant integrating vectors to introduce DNA into plant cells is well known in the art. See, for example, the methods described (Fraley et al., 1985; Rogers et al., 1987). The genetic engineering of cotton plants using Agrobacterium-mediated transfer is described in U.S. Pat. No. 5,004,863 (specifically incorporated herein by reference), while the transformation of lettuce plants is described in U.S. Pat. No. 5,349,124 (specifically incorporated herein by reference). Further, the integration of the Ti-DNA is a relatively precise process resulting in few rearrangements. The region of DNA to be transferred is defined by the border sequences, and intervening DNA is usually inserted into the plant genome as described (Spielmann et al., 1986; Jorgensen et al., 1987).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate construction of vectors capable of expressing various polypeptide coding genes. The vectors described (Rogers et al., 1987), have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

Agrobacterium-mediated transformation of leaf disks and other tissues such as cotyledons and hypocotyls appears to be limited to plants that Agrobacterium naturally infects. Agrobacterium-mediated transformation is most efficient in dicotyledonous plants. Few monocots appear to be natural hosts for Agrobacterium, although transgenic plants have been produced in asparagus using Agrobacterium vectors as described (Bytebier et al., 1987). Therefore, commercially important cereal grains such as rice, corns and wheat must usually be transformed using alternative methods. However, as mentioned above, the transformation of asparagus using Agrobacterium can also be achieved (see, e.g., Bytebier et al., 1987).

A transgenic plant formed using Agrobacterium transformation methods typically contains a single gene on one chromosome. Such transgenic plants can be referred to as being heterozygous for the added gene. However, inasmuch as use of the word “heterozygous” usually implies the presence of a complementary gene at the same locus of the second chromosome of a pair of chromosomes, and there is no such gene in a plant containing one added gene as here, it is believed that a more accurate name for such a plant is an independent segregant, because the added, exogenous gene segregates independently during mitosis and meiosis.

More preferred is a transgenic plant that is homozygous for the added structural gene; i.e., a trarsgenic plant that contains two added genes, one gene at the same locus on each chromosome of a chromosome pair. A homozygous transgenic plant can be obtained by sexually mating (selfing) an independent segregant trarsgenic plant that contains a single added gene, germinating some of the seed produced and analyzing the resulting plants produced for enhanced carboxylase activity relative to a control (native, non-transgenic) or an independent segregant tmnsgenic plant.

It is to be understood that two different tmnsgenic plants can also be mated to produce offspring that contain two independently segregating added, exogenous genes. Selfing of appropriate progeny can produce plants that are homozygous for both added, exogenous genes that encode a polypeptide of interest. Back-crossing to a parental plant and out-crossing with a non-tnansgenic plant are also contemplated.

Transformation of plant protoplasts can be achieved using methods based on calcium phosphate precipitation, polyethylene glycol treatment, electroporation, and combinations of these treatments (see, e.g., Potrykus et al., 1985; Lorz et al., 1985; Fromm et al., 1985; Uchimiya et al., 1986; Callis et al., 1987; Marcotte et al., 1988).

Application of these systems to different plant strains depends upon the ability to regenerate that particular plant strain from protoplasts. Illustrative methods for the regeneration of cereals from protoplasts are described (see, e.g., Fujimura et al., 1985; Toriyama et al, 1986; Yamada et al., 1986; Abdullah et al., 1986).

To transform plant strains that cannot be successfully regenerated from protoplasts, other ways to introduce DNA into intact cells or tissues can be utilized. For example, regeneration of cereals from immature embryos or explants can be effected as described (Vasil, 1988). In addition, “particle gun” or high-velocity microprojectile technology can be utilized (Vasil, 1992).

Using that latter technology, DNA is carried through the cell wall and into the cytoplasm on the surface of small metal particles as described (Klein et al., 1987; Klein et al., 1988; McCabe et al., 1988). The metal particles penetrate through several layers of cells and thus allow the transfomation of cells within tissue explants.

Methods for DNA transformation of plant cells include Agrobacterium-mediated plant transformation, protoplast transformation, gene transfer into pollen, injection into reproductive organs, injection into immature embryos and particle bombardment. Each of these methods has distinct advantages and disadvantages. Thus, one particular method of introducing genes into a particular plant strain may not necessarily be the most effective for another plant strain, but it is well known which methods are useful for a particular plant strain.

There are many methods for introducing transforming DNA segments into cells, but not all are suitable for delivering DNA to plant cells. Suitable methods are believed to include virtually any method by which DNA can be introduced into a cell, such as infection by A. tamefaciens and related Agrobacterium, direct delivery of DNA such as, for example, by PEG-mediated transformation of protoplasts (Ominilleh et al., 1993), by desiccation/inhibition-mediated DNA uptake, by electroporation, by agitation with silicon carbide fibers, by acceleration of DNA coated particles, etc. In certain embodiments, acceleration methods are preferred and include, for example, microprojectile bombardment and the like.

Method of Identifying Plants Comprising an NBS Rice Blast Resistant Allele

The present invention also comprises methods of identifying transgenic or non-transgenic plants that comprise an NBS rice blast resistant allele. The methods comprise isolating DNA or RNA from a cell of the plant and assaying for the presence of such allele using the primers or probes of the present invention.

In one aspect the method is a polymerase chain reaction which employs two primers that amplify the entire coding region of the NBS gene or a unique fragment within one or more of the NBS genes. One primer is located at each end of the region to be amplified. Such primers will normally be between 10 to 30 nucleotides in length and have a preferred length from between 18 to 22 nucleotides. PCR primers can be selected to amplify the entire sequence set forth in SEQ ID NOs. 85, 87, 89, 91 or 93., in which case primers are located at the 5′ and 3′ ends of the illustrated sequences. PCR primers can also be selected to amplify only a part of the sequences set forth in SEQ ID NOs. 85, 87, 89, 91 or 93, in which case at least one of the two primers is located internal to the 5′ and 3′ ends of the illustrated sequences. The smallest such sequence that can be amplified is approximately 50 nucleotides in length (e.g., a forward and reverse primer, both of 20 nucleotides in length, whose location in the sequences in SEQ ID NOs. set forth in SEQ ID NOs. 85, 87, 89, 91 or 93 is separated by at least 10 nucleotides). Any sequence of approximately 50 nucleotides in length that is within the sequences set forth in SEQ ID NOs. 85, 87, 89, 91 or 93 is within the scope of this application.

One primer is called the “forward primer” and is located at the left end of the region to be amplified. The forward primer is identical in sequence to the strand of the DNA set forth in SEQ ID NOs. 85, 87, 89, 91 or 93. The forward primer hybridizes to the strand of the DNA which is complementary to the strand of the DNA set forth in SEQ ID NOs. 85, 87, 89, 91 or 93. With reference to the sequences as oriented in set forth in SEQ ID NOs. 85, 87, 89, 91 or 93, the forward primer primes synthesis of DNA in a leftward to rightward direction.

The other primer is called the “reverse primer” and is located at the right end of the region to be amplified. The reverse primer is complementary in sequence to the strand of the DNA set forth in SEQ ID NOs. 85, 87, 89, 91 or 93. The reverse primer hybridizes to the strand of the DNA set forth in SEQ ID NOs. 85, 87, 89, 91 or 93. With reference to the sequences as oriented in set forth in SEQ ID NOs. 85, 87, 89, 91 or 93, the reverse primer primes synthesis of DNA in a rightward to leftward direction.

Preferably, the primers that are chosen to amplify a sequence within SEQ NOs. 85, 87, 89, 91 or 93 are between 15 to 30 nucleotides in length, more preferably 18 to 25 in length, most preferably between 18 to 22 nucleotides in length.

PCR primers should also be chosen subject to a number of other conditions. PCR primers should be long enough (preferably 15 to 18 nucleotides in length) to minimize hybridization to greater than one region in the genomic template DNA. Primers with long runs of a single base should be avoided, if possible. Primers should preferably have a percent G+C content of between 40 and 60%. If possible, the percent G+C content of the 3′ end of the primer should be higher than the percent G+C content of the 5′ end of the primer. Primers should not contain sequences that can hybridize to another sequence within the primer (i.e., palindromes). Two primers used in the same PCR reaction should not be able to hybridize to one another. Although PCR primers are preferably chosen subject to the recommendations above, it is not necessary that the primers conform to these conditions. Other primers may work, but have a lower chance of yielding good results.

PCR primers that can be used to amplify DNA within a given sequence are preferably chosen using one of a number of computer programs that are available. Such programs choose primers that are optimum for amplification of a given sequence (i.e., such programs choose primers subject to the conditions stated above, plus other conditions that may maximize the functionality of PCR primers). One computer program is the Genetics Computer Group (GCG recently became Accelrys) analysis package which has a routine for selection of PCR primers. There are also several web sites that can be used to select optimal PCR primers to amplify an input sequence. One such web site is http://alces.med.umn.edu/rawprimer.html. Another such web site is http://www-genome.wi.mit.edu/cgi-bin/primer/primer3_www.cgi.

Once PCR primers are chosen, they are used in a PCR reaction. A standard PCR reaction contains a buffer containing 10 mM Tris-HCl (pH 8.3), 50 mM KCl, and 6.0 mM MgCl₂, 200 uM each of dATP, dCTP, dTTP and dGTP, two primers of concentration 0.5 uM each, 7.5 ng/ul concentration of template DNA and 2.5 units of Taq DNA Polymerase enzyme. Variations of these conditions can be used and are well known to those skilled in the art.

The PCR reaction is performed under high stringency conditions. Herein, “high stringency PCR conditions” refers to conditions that do not allow base-pairing mismatches to occur during hybridization of primer to template. Such conditions are equivalent to or comparable to denaturation for 1 minute at 95° C. in a solution comprising 10 mM Tris-HCl (pH 8.3), 50 mM KCl, and 6.0 mM MgCl₂, followed by annealing in the same solution at about 62° C. for 5 seconds.

Successful amplification of the template DNA to produce a PCR product of the correct size (i.e., a size equivalent to the length of the two primers plus the length of DNA between the two primers as set forth in SEQ ID NOS. 85, 87, 89, 91, and 93 is determinative of whether the genome of the plant that is tested contains the targeted NBS rice blast resistant allele. Absence of a PCR product of the correct size indicates that the genome does not contain the targeted NBS rice blast reistant allele.

### REFERENCES

Amante-Bordeos, A., Sitch, L. A., Nelson, R. Damacio, R. D. Oliva, N. P., Aswidinnoor, H., and Leung H. 1992. Transfer of bacterial blight and blast resistance from the tetraploid wild rice Oryza minuta to cultivated rice, Oryza sativa. Theor. Appl. Genet. 84:345-354.

Baker, B., Zambryski, P., Stakawicz, B., and Dinesh-Kumar S. P. 1997. Signaling in plant-microbe interactions. Science 276:726-732.

Bent, A. F. 1996. Plant disease resistance genes: function meets structure. Plant Cell 8:1757-1771.

Bonman, J. M., Vergel De Dios, T. I., and Khin, M. M. 1986. Physiologic specialization of Pyricularia oryzae in the Philippines. Plant Disease 70:767-769.

Bonman, J. M., Khush, G. S., Nelson, R. 1992. Breeding rice for resistance to pests. Annu. Rev. Phytopathol. 30:507-528.

Botella, M. A., Parker, J. E., Frost, L. N., Bittner-Eddy, P. D., Beynon, J. L., Daniels, M. J., Holub, E. B., Jones, J. D. 1998. Three genes of the Arabidopsis RPPI complex resistance locus recognize distinct Peronospora parasitica avirulence determinants. Plant Cell 10: 1847-60.

Bryan, G. T., Wu, K. S., Farrall, L., Jia, Y., Hershey, H. P., McAdams, S. A., Faulk, K. N., Donaldson, G. K., Tarchini, R., Valent, B. 2000. A single amino acid difference distinguishes resistant and susceptible alleles of the rice blast resistance gene Pi-ta. Plant Cell. 12:2033-46.

Chen, D., Zeigler, R. S., Ahn, S. W., Nelson, R. J. 1996. Phenotypic characterization of the rice blast resistance gene Pi-2 (t). Rice. Plant Dis 80:52-56.

Chen, C., Huang, S., Ling, P., Yu, C., Deng, Z., Gmitter, F. G. 1999. A Novel method to clone BAC insert ends based on double digestion. p17, Abstract presented at the Plant and Animal Genome VII, San Diego, Calif. Jan 17-21, 1999.

Dellaporta, S. L., Wood, J., Hicks, J. B. 1984. Maize DNA miniprep. In: Molecular Biology of Plants. A Laboratory Course Manual, M. Russell, Eds. Cold Spring Harbor Laboratory, Cold Spring Harbor, N.Y., pp 36-37

Huang, N., McCouch, S., Mew, T., Parco, A., Guiderdoni, E. 1994. Development of an RFLP map from a doubled haploid population in rice. Rice Genetics Newsletter 11:134-137.

Inukai, T., Mackill, D. J., Bonman, J. M., Sarkarung, S., Zeigler, R., Nelson, R., Takamure, I., and Kinoshita, T. 1992. Blast resistance gene Pi2(t) and Pi-Z may be allelic. Rice Genetics Newsletter 9:90-92.

Jia, Y., McAdams, S. A., Bryan, G. T., Hershey, H. P., and Valent, B. 2000. Direct interaction of resistance gene and avirulence gene products confers rice blast resistance. EMBO J. 19:4004-14.

Mackill, D. J., and Bonman, J. M. 1992. Inheritance of blast resistance in near-isogenic lines of rice. Phytopathology 82:746-749.

McCouch, S. R., Nelson, R. J., Tohme, J., and Zeigler, R. S. 1994. Mapping of blast resistance genes in rice. In Rice Blast Disease, eds Zeigler, R. S., Leong S. A. and Teng, P. S., CAB International. pp 167-186.

Meyers, B. C., Chin, D. B., Shen, K. A., Sivaramakrishnan, S., Lavelle, D. O., Zhang, Z., and Michelmore, R. W. 1998. The major resistance gene cluster in lettuce is highly duplicated and spans several megabases. Plant Cell 10:1817-32.

Michelmore, R. W., and Meyers, B. C. 1998. Clusters of resistance genes in plants evolve by divergent selection and a birth-and-death process. Genome Res 8:1113-30.

Michelmore, R. W., Paran, I., and Kesseli, R. V. 1991. Identification of markers linked to disease-resistance genes by bulked segregant analysis: a rapid method to detect markers in specific genomic regions by using segregating populations. Proc Natl Acad Sci USA. 88:9828-32.

Moffat, A. S. 1994. Mapping the sequence of disease resistance. Science 265:1804-1805.

Ou, S. H. 1985. pp 109-201 in Rice Disease, Ed. 2. The Cambrian News Ltd., UK.

Paran, I., and Michelmore, R. W. 1993. Development of reliable PCR-based markers linked to downy mildew resistance genes in lettuce. Theor Appl Genet 85:985-993.

Sambrook, J., Fritsch, E. F., and Maniatis, T. 1989. Molecular Cloning: A Laboratory Manual, 2nd Ed. Cold Spring Harbor, N.Y.

Sitch, L. A., Amante, A. D., Dalmacio, R. D. and Leung, H. 1989. Oryza minuta, a source of blast and bacterial blight resistance for rice improvement. In Review of Advance in Plant Biotechnology, 1985-1988. Eds: A. Mujeeb-Kazi and LA Sitch, International Maize and Wheat Improvement Center and International Rice Research Institute.

Shizuya, H., Birren, B., Kim, U. J., Mancino, V., Slepak, T., Tachiiri, Y., and Simon, M. 1992. Cloning and stable maintenance of 300-kilobase-pair fragments of human DNA in Escherichia coli using an F-factor-based vector. Proc Natl Acad Sci USA 89:8794-7.

Song, W. Y., Wang, G. L., Chen, L., Zhai, W., Kim, H. K., Holsten, T., Zhu, L. and Ronald, P. 1995. The rice disease resistance gene, Xa21, encodes a receptor-like protein kinase. Science: 270:1804-1806.

Williams, J. G. K., Kubelik, A. R., Livak, K. J., Rafalski, J. A., and Tingey, S. V. 1990. DNA polymorphisms amplified by arbitrary primers are useful as genetic markers. Nucleic Acids Res. 18:6531-6535.

Wang, G. L., Mackill, D. J., Bonman, J. M., McCouch, S. R., and Nelson, R. J. 1994. RFLP mapping of genes conferring complete and partial resistance to blast in a durably resistant rice cultivar. Genetics, 136:1421-1434.

Wang, G. L., Holsten T. E., Song, W. Y., Wang, H. P., Ronald, P.C. 1995. Construction of a rice bacterial artificial chromosome library and identification of clones linked to the Xa21 disease resistance locus. The Plant Journal 7:525-533.

Wang, Z. X., Yano, M., Yamanouchi, U., Iwamoto, M., Monna, L., Hayasaka, H., Katayose, Y., Sasaki, T. 1999. The Pib for rice blast resistance belongs to the nucleotide binding and leucine-rich repeat class of plant disease resistance genes. The Plant J. 19:55-64.

Wei, F., Gobelman-Werner, K., Morroll, S. M., Kurth, J., Mao, L., Wing, R., Leister, D., Schulze-Lefert, P., and Wise, R. P. 1999. The Mla (powdery mildew) resistance cluster is associated with three NBS-LRR gene families and suppressed recombination within a 240-kb DNA interval on chromosome 5S (1HS) of barley. Genetics 153:1929-48.

Yu, Z. H, Mackill, D. J., and Bonman, J. M., and Tanksley, S. D. 1991. Tagging genes for blast resistance in rice via linkage to RFLP markers. Theo. Appl. Genet. 81:471-476.

Yokoo, M., Kikuchi, F., Fujimaki, H. 1978. Breeding of blast resistance lines (BL1-7) from indicajaponica crosses of rice. Jpn J Breed 28:359-385.

TABLE 1
Disease reaction of lines containing Pi9, Pi2 or neither allele to 43 blast
isolates from 14 countries. R stands resistance reaction, MR for medium
or partial resistance reaction, and S for susceptible reaction.

| | | | 75-1-127 | | C101A51 |
|---|---|---|---|---|---|
| Isolate | Country | IR31917 | (Pi9) | CO39 | (Pi2) |
| ML8 | Mali | S | R | S | R |
| ML25 | Mali | S | R | S | S |
| ML33 | Mali | S | R | S | R |
| ML53 | Mali | S | R | S | S |
| 95090B(119) | China | S | R | S | R |
| 96017(138) | China | S | R | S | R |
| SA5ZB13(58) | China | S | R | S | R |
| WAN97ZA13 | China | S | R | S | R |
| 95116AZC13 | China | S | R | S | R |
| 95033ZB15 | China | S | R | S | R |
| ZHONG39ZA7 | China | S | R | S | R |
| (3) | | | | | |
| 36B23 | China | S | R | S | R |
| CHE86056ZB13 | China | S | R | S | R |
| 95097AZC13 | China | S | R | S | R |
| CHE86061ZE13 | China | S | R | S | R |
| 47ZB15(67) | China | S | R | S | R |
| ZHONG79ZC15 | China | S | R | S | R |
| 87024ZG1 | China | S | R | S | MR |
| 78334ZA27(7) | China | S | R | S | R |
| ZHONGSZC7 | China | S | R | S | R |
| CH64 | China | S | R | S | R |
| 97-4-1 | China | R | R | S | R |
| 97-104-2 | China | S | R | S | R |
| 97-5-1 | China | S | R | S | R |
| 97-102-2 | China | S | R | S | R |
| 97-55-2 | China | S | R | S | R |
| 54-04 | China | S | R | S | R |
| 10-18-14 | China | S | R | S | S |
| TH4 | Thailand | S | R | S | S |
| PO6-6 | Philip- | S | R | S | R |
| | pines | | | | |
| C9240-1 | Philip- | S | R | S | S |
| | pines | | | | |
| PH9 | Philip- | S | R | S | R |
| | pines | | | | |
| DB24 | Burundi | S | R | S | S |
| CD101 | Ivory | S | R | S | R |
| | Coast | | | | |
| GUY11 | France | S | R | S | S |
| ES5 | Spain | S | R | S | R |
| ES6 | Spain | S | R | S | R |
| IT10 | Italy | S | R | S | R |
| PR3 | Portugal | MR | R | S | R |
| US30 | USA | R | R | S | R |
| CL6 | Colombia | S | R | S | R |
| KJ201 | South | S | R | S | R |
| | Korea | | | | |
| KI1117 | South | S | R | S | R |
| | Korea | | | | |

TABLE 2

| | NBS1 | NBS2 | NBS3 | NBS4 | NBS5 | NBS6 | PIB |
|---|---|---|---|---|---|---|---|
| NBS 1 | | 57.75 | 62.88 | 49.69 | 62.78 | 59.06 | 32.05 |
| | 66.23 | 69.63 | 57.35 | 69.53 | 67.11 | 42.14 | |
| NBS2 | | | 55.36 | 45.59 | 55.16 | 48.86 | 33.40 |
| | | | 63.76 | 53.26 | 63.46 | 58.45 | 43.87 |
| NBS3 | | | | 51.72 | 98.06 | 62.88 | 32.95 |
| | | | | 59.49 | 98.06 | 72.22 | 44.77 |
| NBS4 | | | | | 51.67 | 92.90 | 28.81 |
| | | | | | 59.46 | 95.12 | 38.52 |
| NBS5 | | | | | | 62.66 | 32.48 |
| | | | | | | 72.00 | 44.22 |
| NBS6 | | | | | | | 31.53 |
| | | | | | | | 41.18 |

TABLE 4
Pi9 Primers

| | |
|---|---|
| Name | Seq |

| | |
|---|---|
| 07.0kb-F1 | TCCCAAATCTCAGGTGTCTT |
| 7.0kb-R1 | ATCAGCAGGCGGCAAACT |
| 7.0kbNBS-F1 | GTAGGTACATCAAGGACGAG |
| 7.0kbNBS-R1 | AGCATATCTTTGAGCATTTG |
| 7.0kbNBS-F2 | ACTGTTGTAGCGGAGGAGA |
| 7.0kbNBS-R2 | TTTCCATTGTRGGCGAGGTG |
| N2AR-F | 5′ATGTCAGCCAAAATCAATCA |
| N2AR-R | 5′GGAAGGGGACAAGGACAATA |
| N2A19N-F | 5′CTTTTCGTGGGNGGGGTTA |
| N2A19N-R | 5′GGTGAGTGATGACAGCAACA |
| B3NlC-F | 5′CAGCTTTGAGGACATTCG 3′ |
| B3N1C-R | 5′TTTTGACCCCAGACGACCAG 3′ |
| B3N2A-F | 5′GAGAGGGGATGGACAAAGAG 3′ |
| B3N2A-R | 5′GGAAAACGAAACGGTAGATT 3′ |
| B3N1CR-R | 5′TGCCGCCTGCTCGTCCTC |
| B3N1CR-F | 5′GGTTGTTTTCCCTTGTCC |
| 75-19-F1 | 5′AAGGAAGATGAGCCGTGAT |
| 75-19-R1 | 5′TGATGCGTGATGATTTTGTA |
| 75-19-F2 | TCTGATGTCCTCTGAACTGA |
| 75-19-R2 | ACTGCCTCTGCTGTTGTTGA |
| 19RF-F | GGTCTGGCACTATTTTTACTTT |
| 19RT-R | GCAAAAGGAACTGATAAGAT |
| 74RF-F | ATCTCATTTTTAGGTTCTGTCG |
| 74RF-R | CAAGCAGCCACCACCATCTC |
| 70RF-F | CGACCGAACCGCCTTTAG |
| 70RF-R | TCAACGAAGAAGAGATGTAG |
| 4RF-F | AATACACACCCCAATCATACTG |
| 4RF-R | AAAAACAAGACGGCAGAACAGA |
| 19RF-F1 | AAGAAGATAATGGAATGGGG |
| 19RF-R1 | GAGAGATTGCAGAGAAGAGA |
| 19S-16 | TTTCCTTCTTGTAACCTGAT |
| 19N-29 | CTGCTTGCTATTCGTTCATC |
| W8F-1 | <5′taacagttctcccaatctcc> |
| W8R1 | <5′ccggactaagtactggct> |
| V14F-3, | 5′GCCATGTTGCTGCGGAAAAT |
| V14R-3, | 5′AAACTTAGGGCATTCAATCC |
| A114F-2, | 5′TGGTGCACTCAGAAAGAAT |
| V14F-4, | 5′CGCCATGTTGCTGCGGAA |
| V14R-4, | 5′AGATCCCGCCACTAAACTTA |
| A114F-3, | 5′CTTGTTGGTATGAGTATTCT |
| A114R-2, | 5′GCAGTGTCATCTTGTCTCC |
| 19LEND-F1 | TTGAATACAGTGCTAAAGTG |
| 19LEND-R1 | GTATGACAATGGATGGAGAA |
| 19LEND-F2 | TAGCAAGGGATGGGAGCAA |
| 19LEND-R2 | TAGGTGCACAGGAAGAGAAT |
| 19LEND-F3 | TCGGTATTTGTTTGATTGGA |
| 19LEND-R3 | CGCGTGATATTCTTGACTGT |
| 19REND-F1 | ATTAGGCACCCCAGGCTTTA |
| 19REND-R1 | TCCTTGTGGCGATTTGTATT |
| 19REND-F2 | TCCAGGTAAAGAGAAAAGT |
| 19REND-R2 | TTGTACTAAATCAGAAGCAC |
| 19REND-F3 | TGTTGGCATCTTTTATCTGA |
| 19REND-R3 | TAGCTGCTATTTGTGATGTA |
| 7.0Prom-F | GCACGCTTCTGTAACTCCA |
| 7.0Prom-R | ATTAGATTTGGCGATTATGC |
| 7.0-3Prim-F | TACACACCCCAATCATACTG |
| 7.0-3Prim-R | GCATCAGCAGGCGGCAAACT |
| 74REND-F1 | ATAGAGTGAGATGGAGAAGA |
| 74REND-R1 | TTAAAGCCCGTCACCGATAG |
| 74REND-F2 | GCACCCCAGGCTTTACACT |
| 74REND-R2 | GGGCNTAATTTCAGTTCTTT |
| 74LEND-F1 | GTACTGGTGTGATTATGGTG |
| 74LEND-R1 | AAAAATACAAAGAAAAACC |
| 31REND-F1 | CAGGCTTTACACTTTATGC |
| 31REND-R1 | CGATACGTGTTTTTCTGGAC |
| 31REND-F2 | GCACATTAACAACAAAGAAA |
| 31REND-R2 | ATGTTCAGTTGGAGAGTAGC |
| 7.0kbNBS-F1 | GTAGGTACATCAAGGACGAG |
| NBS/LRR-R1 | AGGTGTTCGCCCCGCAGGT |
| NBS/LRR-F2 | CACTGTTGTAGCGGAGGAGA |
| NBS/LRR-R2 | CAGTACGCGATTTTCATTGTTC |
| Pst.seq.1 | CGTGCTGATGTGATGCTTCTA |
| 19-7-F1 | ttcttcgtctttatcgcaac |
| 3Prime-R1 | GGTGCAGCGGGGAGGAGGT |
| BeforeH3-R | GGTGCCCTCCTCAAATCTTATC |
| 12L-R-F | TTGGCGTCTTGAGGAGTCGTAT |
| 12L-R-R | GGCGGTGGTGGCGAGGAGTT |
| AfterH3-F | GGTGCAGCGGGGAGGAGGT |
| AfterH3-F1 | ATGTGCTGCAAGAGGAAGTGAA |
| 195F-1 | TTGCTCCATCTTCCTCTGTT |
| 195R-1 | ATGGTCCTTTACTTTATTG |
| 195F-F | ATACAGACCAGAGAAAGAAAAA |
| 195F-R | ACAGAGGAAGATGGAGCAAAGT |

What is claimed is:

1. An isolated nucleic acid comprising a nucleotide sequence that encodes an NBS protein which comprises an amino acid sequence selected from the group consisting of SEQ ID NO. 85, SEQ ID NO. 87, SEQ ID NO 89, SEQ ID NO. 91, SEQ ID NO. 93, and SEQ ID NO. 95.

2. The isolated nucleic acid of claim 1 wherein said nucleotide sequence encodes an NBS 1, NBS2, or NBS3 protein.

3. An isolated nucleic acid that hybridizes under stringent conditions to the sequence set forth in SEQ ID NO. 84, 86, 88, 90, 92, or 94 or a sequence which is complementary thereto, wherein said isolated nucleic acid is at least 15 nucleotides in length.

4. The isolated nucleic acid of claim 3 wherein said nucleic acid is at least 50 nucleotides in length.

5. The isolated nucleic acid of claim 3 wherein the isolated nucleic acid is of the same length as SEQ ID NO. 84, SEQ ID NO. 86, SEQ ID NO. 88, SEQ ID NO. 90, SEQ ID NO. 92, or SEQ ID NO. 94, respectively.

6. A DNA construct comprising in the 5′ to 3′ direction: a promoter regulatory element, a nucleic acid encoding an NBS protein from a rice plant resistant to infection with Magnaporthe grisea, and a transcriptional terminator sequence, wherein either the promoter regulatory element or the transcriptional terminator sequence is not naturally associated with said nucleic acid.

7. The DNA construct of claim 6 wherein the NBS protein is NBS1, NBS2, or NBS3, and the nucleic acid comprises a sequence which encodes the amino acid sequence set forth in SEQ ID NO. 85, SEQ ID NO. 87, or SEQ ID NO. 89, respectively.

8. A plant cell stably incorporating into its genome the nucleic acid construct of claim 6.

9. The plant cell of claim 8 wherein the nucleic acid construct comprises a nucleotide sequence which encodes the amino acid sequence set forth in SEQ ID NO. 84, SEQ ID NO. 86, or SEQ ID NO. 88

10. The plant cell of claim 8 wherein said promoter regulatory element is a constitutive promoter or an inducible promoter.

11. The plant cell of claim 8 wherein the plant cell is from a rice plant, a wheat plant or a barley plant.

12. A transgenic plant produced from the plant cell of claim 8.

13. The transgenic plant of claim 12 wherein said transgenic plant is resistant to infection by Magnaporthe grisea.

14. The transgenic plant of claim 12 wherein said transgenic plant is a transgenic rice plant, a transgenic wheat plant or a transgenic barley plant.

15. Seed and progeny produced from the plant cell of claim 12.

16. A method for the production of a plant having an NBS rice blast resistant allele, wherein said plant is a member of the grass family, comprising: (A) crossing a first plant having an NBS rice blast resistant allele with a second plant having an NBS rice blast resistant allele to produce a population of plants; (B) screening said population of plants for a member having an NBS resistant allele with a nucleic acid molecule capable of specifically hybridizing to SEQ ID NO. 84, 86, 88, 90, 92, or 94 or a complement thereof or a fragment thereof having at least 15 nucleotides; and, (C) selecting said member for further crossing and selection.

17. The method of claim 16 wherein said plant is a rice plant, a wheat plant or a barley plant.

18. The method of claim 16 wherein said screening is achieved using a polymerase chain reaction and a primer set which amplifies all or a portion of SEQ ID NO. 84, 86, 88, 90, 92, or 94.

19. The method of claim 16 wherein said screening is achieved using a probe that binds under stringent conditions to a sequence of 15 contiguous nucleotides within SEQ ID NO. 84, 86, 88, 90, 92, or 94.

20. A method of identifying a plant comprising an NBS rice blast resistant allele, comprising:
isolating DNA or RNA from said plant, and
assaying for the presence of one or more NBS rice blast resistant alleles by a polymerase chain reaction (PCR) which employs a first primer that is at least 15 nucleotides in length and comprises a sequence which is identical to a contiguous sequence in SEQ ID NO. 84, SEQ ID NO 86, SEQ ID NO. 88, SEQ ID NO. 90, SEQ ID NO. 92, or SEQ ID NO 94 and a second primer which is the reverse complement of a contiguous sequence in SEQ ID NO. 84, SEQ ID NO 86, SEQ ID NO. 88, SEQ ID NO. 90, SEQ ID NO. 92, or SEQ ID NO 94, and wherein the product that is produce by said PCR is at least 50 nucleotides in length.
